(19) United States
(12) Patent Application Publication
(10) Pub. No.: US 2023/0382969 A1
(43) Pub. Date: Nov. 30, 2023

(54) INTERLEUKIN-2 PROPROTEINS AND USES THEREOF

(71) Applicant: Regeneron Pharmaceuticals, Inc., Tarrytown, NY (US)

(72) Inventors: Supriya PATEL, Elmsford, NY (US); Tong ZHANG, New Rochelle, NY (US); Jiaxi WU, Pleasantville, NY (US); Aaron CHANG, White Plains, NY (US); Eric SMITH, New York, NY (US); Chia-Yang LIN, Scarsdale, NY (US); Samuel DAVIS, New York, NY (US)

(73) Assignee: Regeneron Pharmaceuticals, Inc., Tarrytown, NY (US)

(21) Appl. No.: 18/324,380

(22) Filed: May 26, 2023

Related U.S. Application Data
(60) Provisional application No. 63/501,434, filed on May 11, 2023.
(60) Provisional application No. 63/386,999, filed on Dec. 12, 2022.
(60) Provisional application No. 63/355,375, filed on Jun. 24, 2022.
(60) Provisional application No. 63/349,078, filed on Jun. 4, 2022.
(60) Provisional application No. 63/346,593, filed on May 27, 2022.

(51) Int. Cl. C07K 14/55; A61P 35/00

(52) U.S. Cl. CPC C07K 14/55; A61P 35/00

(57) ABSTRACT

The present disclosure provides IL2 proproteins comprising an IL2 moiety that is masked with an IL2Rα moiety and a protease-cleavable linker, configured such that the IL2Rα moiety is released from the IL2 moiety upon the action of a protease, e.g., at a tumor site. The IL2 proproteins optionally further comprise a targeting moiety, e.g., a targeting moiety that recognizes a tumor-associated antigen and directs the proprotein to a tumor site. The disclosure further provides pharmaceutical compositions comprising the IL2 proproteins, and methods of use of the IL2 proproteins in therapy, as well as nucleic acids encoding the IL2 proproteins, recombinant cells that express the IL2 proproteins and methods of producing the IL2 proproteins.

### 1. CROSS REFERENCE TO RELATED APPLICATIONS

This application claims the priority benefit of U.S. provisional application No. 63/346,593, filed May 27, 2022, U.S. provisional application No. 63/349,078, filed Jun. 4, 2022, U.S. provisional application No. 63/355,375, filed Jun. 24, 2022, U.S. provisional application No. 63/386,999, filed Dec. 12, 2022, and U.S. provisional application No. 63/501,434, filed May 11, 2023, the contents of each of which are incorporated herein in their entireties by reference thereto.

### 2. SEQUENCE LISTING

The instant application contains a Sequence Listing which has been submitted electronically and is hereby incorporated by reference in its entirety. Said copy, created on May 9, 2023, is named RGN-021US_SL.xml and is 316,721 bytes in size.

### 3. BACKGROUND

Interleukin 2 (IL-2 or IL2) is a pluripotent cytokine produced primarily by CD4+ helper T cells. It stimulates the proliferation and differentiation of T cells, induces the generation of cytotoxic T lymphocytes (CTLs) and the differentiation of peripheral blood lymphocytes to cytotoxic cells and lymphokine-activated killer (LAK) cells, promotes cytokine and cytolytic molecule expression by T cells, facilitates the proliferation and differentiation of B-cells and the synthesis of immunoglobulin by B-cells, and stimulates the generation, proliferation and activation of natural killer (NK) cells (see Waldmann, 2009, Nat Rev Immunol 6:595-601 and Malek, 2008, Annu Rev Immunol 26:453-79).

Due to its pleotropic effects, IL2 is not optimal for inhibiting tumor growth. The use of IL2 as an antineoplastic agent has been limited by the serious toxicities that accompany the doses necessary for a tumor response. Proleukin® (marketed by Prometheus Laboratories, San Diego, Calif.), is a recombinant form of IL2 that is approved for the treatment of metastatic melanoma and metastatic renal cancer, but its side effects are so severe that its use is only recommended in a hospital setting with access to intensive care. Patients receiving high-dose IL2 treatment frequently experience severe cardiovascular, pulmonary, renal, hepatic, gastrointestinal, neurological, cutaneous, haematological and systemic adverse events, which require intensive monitoring and in-patient management. The major side effect of IL2 therapy is vascular leak syndrome (VLS), which leads to the accumulation of interstitial fluid in the lungs and liver resulting in pulmonary edema and liver damage. There is no treatment for VLS other than withdrawal of IL2. Low-dose IL2 regimens have been tested in patients to avoid VLS, however, at the expense of suboptimal therapeutic results. It has been shown that IL2-induced pulmonary edema resulted from direct binding of IL2 to lung endothelial cells, which express low to intermediate levels of functional high affinity IL2 receptors (Krieg et al., 2010, Proc Nat Acad Sci USA 107:11906-11).

A variety of IL2 variants and prodrugs have been generated with the aim of reducing the toxicity of IL2 cancer therapy. However, it has been surprisingly discovered that such molecules have poor therapeutic indices for cancer therapy. For example, the PEGylated IL2 prodrug bempegaldesleukin failed to improve on the therapeutic efficacy of a PD1 checkpoint inhibitor in melanoma patients in phase 3 clinical studies (Mullard, 2022, Nature Reviews Drug Discovery 21:327 (doi: https://doi.org/10.1038/d41573-022-00069-3).

Thus, there is a need in the art for novel IL2 therapies with improved therapeutic efficacy and safety profiles.

### 4. SUMMARY

The present disclosure relates to IL2 proproteins that are activated by proteases, e.g., proteases expressed in the tumor environment.

The IL2 proproteins comprise an IL2 moiety that is masked by an IL2Rα moiety, configured so the mask is released following cleavage by a protease. The IL2 proproteins preferably further comprise a targeting moiety that directs the IL2 proprotein to a particular tissue or cell type.

In certain embodiments, the IL2 proproteins of the disclosure comprise two polypeptide chains, each comprising, from N- to C-terminus, an Fc domain, an optional linker (which if present is preferably a non-cleavable linker), an IL2Rα moiety, a protease-cleavable linker and an IL2 moiety. The IL2 proproteins may further comprise, e.g., N-terminal to one or both Fc domains, a targeting moiety (or a component thereof, e.g., one chain of a Fab). The targeting moiety comprises an antigen-binding site (“ABS”) that can, for example, bind to a target molecule present on the tumor surface (e.g., a tumor associated antigen) or other component in the tumor microenvironment (e.g., extracellular matrix (“ECM”) or tumor lymphocytes).

Exemplary IL2 moieties that can be used in the IL2 proproteins of the disclosure are described in Section 6.3.

Exemplary IL2Rα moieties that can be used in the IL2 proproteins of the disclosure are described in Section 6.4.

Protease-cleavable linkers that can be used in the IL2 proproteins of the disclosure are described in Section 6.5.

Non-cleavable linkers that can be used in the IL2 proproteins of the disclosure are described in Section 6.6.

Targeting moieties that can be used in the IL2 proproteins of the disclosure are described in Section 6.7 and targeting moiety formats are disclosed in Section 6.8.

Fc domains that can be incorporated into the IL2 proproteins of the disclosure are described in Section 6.9.

Exemplary IL2 proproteins of the disclosure are described in Section 6.2 and numbered embodiments 1 to 157.

The disclosure further provides nucleic acids encoding the IL2 proproteins of the disclosure. The nucleic acids encoding the IL2 proproteins can be a single nucleic acid (e.g., a vector encoding all polypeptide chains of an IL2 proprotein) or a plurality of nucleic acids (e.g., two or more vectors encoding the different polypeptide chains of an IL2 proprotein). The disclosure further provides host cells and cell lines engineered to express the nucleic acids and IL2 proproteins of the disclosure. The disclosure further provides methods of producing an IL2 proprotein of the disclosure. Exemplary nucleic acids, host cells, and cell lines, and methods of producing an IL2 proprotein are described in Section 6.10 and specific embodiments 158 to 160.

The disclosure further provides pharmaceutical compositions comprising the IL2 proproteins of the disclosure. Exemplary pharmaceutical compositions are described in Section 6.11 and numbered embodiment 161.

Further provided herein are methods of using the IL2 proproteins and the pharmaceutical compositions of the disclosure, e.g., for treating cancer. Exemplary methods are described in Section 6.12 and numbered embodiments 162 to 200.

### 5. BRIEF DESCRIPTION OF THE FIGURES

FIGS. 1A-1B. FIG. 1A is an illustration of an exemplary targeted IL2 proprotein of the disclosure, with both polypeptide chains comprising the Targeting Moiety—Fc Domain—Linker—IL2Rα Moiety—Cleavable Linker—IL2 Moiety format. Although the targeting moieties in FIG. 1A are illustrated as Fabs, the targeting moieties can be in other formats, e.g., scFvs or other formats described in Section 6.8. FIGS. 1A-1 and 1A-2 illustrate a close-up view of an embodiment of Linkers B and D, respectively comprising a spacer (B1, B2 and D1, D2, respectively) on either side of a cleavable substrate. The number of substrate and spacer sequences is for illustrative purposes only, and it is expected that the protease cleavable linkers will typically have multiple substrate and spacer sequences as detailed in Section 6.5. FIG. 1B illustrates the mechanism of activation of an exemplary targeted IL2 proprotein according to FIG. 1A.

FIG. 2 is an image of a gel from SDS-PAGE analysis of recombinant protease digestion of several protease-cleavable linkers in the Targeting Moiety—IL2Rα Moiety—Linker—IL2 Moiety format.

FIG. 3 is an image of a gel from SDS-PAGE analysis of recombinant protease digestion of several protease-cleavable linkers in the Targeting Moiety—IL2Rα Moiety—Linker—IL2 Moiety format.

FIGS. 4A-4C relate to a CD20-targeted IL2 proprotein. FIG. 4A shows that protease mediated cleavage of IL2 from CD20-IL2Rα-PCL-IL2 leads to improved activity in YT/STAT5 luc/CL4 luciferase assay. FIG. 4B is an illustration of IL2 engaged with an IL2 receptor and FIG. 4C is an illustration of an IL2 proprotein that does not engage with an IL2 receptor.

FIGS. 5A-5N show the in vivo efficacy of tumor targeted IL2Rα-IL2 with a protease-cleavable linker.

FIGS. 6A-6B show the stability of a protease-cleavable linker in vivo.

FIG. 7 illustrates study design to determine the contribution of tumor targeting in the anti-tumor efficacy of IL2 proproteins with a protease-cleavable linker in a MC38/hCD20 model.

FIG. 8 shows changes in tumor volume following treatment in accordance with the study design of FIG. 7.

FIGS. 9A-9G show that CD20 targeted IL2Rα-IL2 proteins with cleavable linkers showed significantly better anti-tumor efficacy and tumor free survival compared to non-targeted IL2Rα-IL2 proteins with cleavable linker and CD20 targeted IL2Rα-IL2 with a non-cleavable G4S linker (SEQ ID NO: 1) in a MC38/hCD20 mouse model.

### 6. DETAILED DESCRIPTION

### 6.1. Definitions

As used herein, the following terms are intended to have the following meanings:

ABD chain, targeting moiety chain: Targeting moieties and antigen binding sites (ABD's) within them can exist as one (e.g., in the case of an scFv or scFab) polypeptide chain or form through the association of more than one polypeptide chains (e.g., in the case of a Fab or an Fv). As used herein, the terms “ABD chain” and “targeting moiety chain” refer to all or a portion of an ABD or targeting moiety that exists on a single polypeptide chain. The use of the term “ABD chain” or “targeting moiety chain” is intended for convenience and descriptive purposes only and does not connote a particular configuration or method of production. Further, the reference to an ABD or targeting moiety when describing an IL2 proprotein encompasses an ABD chain or targeting moiety chain unless the context dictates otherwise. Thus, when describing an IL2 proprotein in which an Fc domain is operably linked to a targeting moiety, the Fc domain may be covalently linked directly or indirectly (e.g., via a linker) through a peptide bond to, e.g., (1) a first ABD or targeting moiety chain of a Fab or Fv (with the other components of the Fab or Fv on a second, associated ABD or targeting moiety chain) or (2) an ABD or targeting moiety chain containing an scFv or scFab.

About, Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

Activate, activation: The terms “activation”, “activation”, and the like in conjunction with an IL2 proprotein of the disclosure refers to the protease-mediated enzymatic cleavage of a protease-cleavable linker that results in the unmasking or release of an IL2 moiety from an IL2Rα moiety.

And, or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelized antibodies, chimeric antibodies, bispecific or multispecific antibodies and anti-idiotypic (anti-id) antibodies. The antibodies can be of any isotype/class (e.g., IgG, IgE, IgM, IgD, IgA and IgY) or subclass (e.g., IgG1, IgG2, IgG3, IgG4, IgA1 and IgA2). Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding domain or amino-terminus of the antibody. The N-terminus is a variable region and at the C-terminus is a constant region; the CH3 and CL domains represent the carboxy-terminus of the heavy and light chain, respectively, of natural antibodies. For convenience, and unless the context dictates otherwise, the reference to an antibody also refers to antibody fragments as well as engineered antibodies that include non-naturally occurring antigen-binding domains and/or antigen-binding domains having non-native configurations.

Antigen-binding domain: The term “antigen-binding domain” or “ABD” as used herein refers to a portion of an antibody or antibody fragment (e.g., a targeting moiety) that has the ability to bind to an antigen non-covalently, reversibly and specifically. Examples of an antibody fragment that can comprise an ABD include, but are not limited to, a single-chain Fv (scFv), a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989, Nature 341:544-546), which consists of a VH domain; and an isolated complementarity determining region (CDR). Thus, the term “antibody fragment” encompasses both proteolytic fragments of antibodies (e.g., Fab and F(ab)₂ fragments) and engineered proteins comprising one or more portions of an antibody (e.g., an scFv). Antibody fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology 23: 1126-1136).

Associated: The term “associated” in the context of an IL2 proprotein refers to a functional relationship between two or more polypeptide chains. In particular, the term “associated” means that two or more polypeptides are associated with one another, e.g., non-covalently through molecular interactions or covalently through one or more disulfide bridges or chemical cross-linkages, so as to produce a functional IL2 proprotein. Examples of associations that might be present in an IL2 proprotein of the disclosure include (but are not limited to) associations between Fc domains to form an Fc region (homodimeric or heterodimeric as described in Section 6.9), associations between VH and VL regions in a Fab or Fv, and associations between CH1 and CL in a Fab.

Cancer: The term “cancer” refers to a disease characterized by the uncontrolled (and often rapid) growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, adrenal gland cancer, autonomic ganglial cancer, biliary tract cancer, bone cancer, endometrial cancer, eye cancer, fallopian tube cancer, genital tract cancers, large intestinal cancer, cancer of the meninges, oesophageal cancer, peritoneal cancer, pituitary cancer, penile cancer, placental cancer, pleura cancer, salivary gland cancer, small intestinal cancer, stomach cancer, testicular cancer, thymus cancer, thyroid cancer, upper aerodigestive cancers, urinary tract cancer, vaginal cancer, vulva cancer, lymphoma, leukemia, lung cancer and the like, e.g., any TAA-positive cancers of any of the foregoing types.

Complementarity Determining Region: The terms “complementarity determining region” or “CDR,” as used herein, refer to the sequences of amino acids within antibody variable regions which confer antigen specificity and binding affinity. For example, in general, there are three CDRs in each heavy chain variable region (e.g., CDR-H1, CDR-H2, and CDR-H3) and three CDRs in each light chain variable region (CDR-L1, CDR-L2, and CDR-L3). The precise amino acid sequence boundaries of a given CDR can be determined using any of a number of well-known schemes, including those described by Kabat et al., 1991, “Sequences of Proteins of Immunological Interest,” 5th Ed. Public Health Service, National Institutes of Health, Bethesda, MD (“Kabat” numbering scheme), AI-Lazikani et al., 1997, JMB 273:927-948 (“Chothia” numbering scheme) and ImMunoGenTics (IMGT) numbering (Lefranc, 1999, The Immunologist 7:132-136; Lefranc et al., 2003, Dev. Comp. Immunol. 27:55-77 (“IMGT” numbering scheme). For example, for classic formats, under Kabat, the CDR amino acid residues in the heavy chain variable domain (VH) are numbered 31-35 (CDR-H1), 50-65 (CDR-H2), and 95-102 (CDR-H3); and the CDR amino acid residues in the light chain variable domain (VL) are numbered 24-34 (CDR-L1), 50-56 (CDR-L2), and 89-97 (CDR-L3). Under Chothia, the CDR amino acids in the VH are numbered 26-32 (CDR-H1), 52-56 (CDR-H2), and 95-102 (CDR-H3); and the amino acid residues in VL are numbered 26-32 (CDR-L1), 50-52 (CDR-L2), and 91-96 (CDR-L3). By combining the CDR definitions of both Kabat and Chothia, the CDRs consist of amino acid residues 26-35 (CDR-H1), 50-65 (CDR-H2), and 95-102 (CDR-H3) in human VH and amino acid residues 24-34 (CDR-L1), 50-56 (CDR-L2), and 89-97 (CDR-L3) in human VL. Under IMGT the CDR amino acid residues in the VH are numbered approximately 26-35 (CDR-H1), 51-57 (CDR-H2) and 93-102 (CDR-H3), and the CDR amino acid residues in the VL are numbered approximately 27-32 (CDR-L1), 50-52 (CDR-L2), and 89-97 (CDR-L3) (numbering according to “Kabat”). Under IMGT, the CDR regions of an antibody can be determined using the program IMGT/DomainGap Align.

Effector Function: The term “effector function” refers to an activity of an antibody molecule that is mediated by binding through a domain of the antibody other than the antigen-binding domain, usually mediated by binding of effector molecules. Effector function includes complement-mediated effector function, which is mediated by, for example, binding of the C1 component of the complement to the antibody. Activation of complement is important in the opsonization and lysis of cell pathogens. The activation of complement also stimulates the inflammatory response and may also be involved in autoimmune hypersensitivity. Effector function also includes Fc receptor (FcR)-mediated effector function, which may be triggered upon binding of the constant domain of an antibody to an Fc receptor (FcR). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. An effector function of an antibody may be altered by altering, e.g., enhancing or reducing, the affinity of the antibody for an effector molecule such as an Fc receptor or a complement component. Binding affinity will generally be varied by modifying the effector molecule binding site, and in this case it is appropriate to locate the site of interest and modify at least part of the site in a suitable way. It is also envisaged that an alteration in the binding site on the antibody for the effector molecule need not alter significantly the overall binding affinity but may alter the geometry of the interaction rendering the effector mechanism ineffective as in non-productive binding. It is further envisaged that an effector function may also be altered by modifying a site not directly involved in effector molecule binding, but otherwise involved in performance of the effector function.

Epitope: An epitope, or antigenic determinant, is a portion of an antigen recognized by an antibody or other antigen-binding moiety as described herein. An epitope can be linear or conformational.

Fab: The term “Fab” refers to a pair of polypeptide chains, the first comprising a variable heavy (VH) domain of an antibody operably linked (typically N-terminal to) to a first constant domain (referred to herein as C1), and the second comprising variable light (VL) domain of an antibody N-terminal operably linked (typically N-terminal) to a second constant domain (referred to herein as C2) capable of pairing with the first constant domain. In a native antibody, the VH is N-terminal to the first constant domain (CH1) of the heavy chain and the VL is N-terminal to the constant domain of the light chain (CL). The Fabs of the disclosure can be arranged according to the native orientation or include domain substitutions or swaps that facilitate correct VH and VL pairings. For example, it is possible to replace the CH1 and CL domain pair in a Fab with a CH3-domain pair to facilitate correct modified Fab-chain pairing in heterodimeric molecules. It is also possible to reverse CH1 and CL, so that the CH1 is attached to VL and CL is attached to the VH, a configuration generally known as Crossmab. The term “Fab” encompasses single chain Fabs.

Fc Domain and Fc Region: The term “Fc domain” refers to a portion of the heavy chain that pairs with the corresponding portion of another heavy chain. The term “Fc region” refers to the region of formed by association of two heavy chain Fc domains. The two Fc domains within the Fc region may be the same or different from one another. In a native antibody the Fc domains are typically identical, but one or both Fc domains might be modified to allow for heterodimerization, e.g., via a knob-in-hole interaction.

Fv: The term “Fv” refers to the minimum antibody fragment derivable from an immunoglobulin that contains a complete target recognition and binding site. This region consists of a dimer of one heavy and one light chain variable domain in a tight, noncovalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target. The reference to a VH-VL dimer herein is not intended to convey any particular configuration. When present on a single polypeptide chain (e.g., a scFv), the VH and be N-terminal or C-terminal to the VL.

Half Antibody: The term “half antibody” refers to a molecule that comprises at least one Fc domain and can associate with another molecule comprising an Fc through, e.g., a disulfide bridge or molecular interactions. A half antibody can be composed of one polypeptide chain or more than one polypeptide chains (e.g., the two polypeptide chains of a Fab). An example of a half antibody is a molecule comprising a heavy and light chain of an antibody (e.g., an IgG antibody). Another example of a half antibody is a molecule comprising a first polypeptide comprising a VL domain and a CL domain, and a second polypeptide comprising a VH domain, a CH1 domain, a hinge domain, a CH2 domain, and a CH3 domain, wherein said VL and VH domains form an ABD. Yet another example of a half antibody is a polypeptide comprising an scFv domain, a CH2 domain and a CH3 domain. The IL2 proproteins of the disclosure typically comprise two half antibodies, each comprising an Fc domain, an IL2Rα moiety C-terminal to the Fc domain, a protease-cleavable linker C-terminal to the IL2Rα moiety, and an IL2 moiety C-terminal to the protease-cleavable linker. One or both half antibodies in the IL2 proproteins may further comprise a targeting moiety, e.g., N-terminal to the Fc domain.

The term “half antibody” is intended for descriptive purposes only and does not connote a particular configuration or method of production. Descriptions of a half antibody as a “first” half antibody, a “second” half antibody, a “left” half antibody, a “right” half antibody or the like are merely for convenience and descriptive purposes.

Host cell or recombinant host cell: The terms “host cell” or “recombinant host cell” refer to a cell that has been genetically-engineered, e.g., through introduction of a heterologous nucleic acid. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A host cell may carry the heterologous nucleic acid transiently, e.g., on an extrachromosomal heterologous expression vector, or stably, e.g., through integration of the heterologous nucleic acid into the host cell genome. For purposes of expressing a IL2 proprotein of the disclosure, a host cell is preferably a cell line of mammalian origin or mammalian-like characteristics, such as monkey kidney cells (COS, e.g., COS-1, COS-7), HEK293), baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells, or derivatives and/or engineered variants thereof. The engineered variants include, e.g., derivatives that grow at higher density than the original cell lines and/or glycan profile modified derivatives and and/or site-specific integration site derivatives.

Linker: The term “linker” as used herein refers to a protease-cleavable linker or a non-cleavable linker.

Non-cleavable linker: A non-cleavable linker refers to a peptide whose amino acid sequence lacks a substrate sequence for a protease, e.g., a protease as described in Section 6.5.1, that recognizes and cleaves a specific sequence motif, e.g., a substrate as described in Section 6.5.2.

Operably linked: The term “operably linked” refers to a functional relationship between two or more peptide or polypeptide domains or nucleic acid (e.g., DNA) segments. In the context of a fusion protein or other polypeptide, the term “operably linked” means that two or more amino acid segments are linked so as to produce a functional polypeptide. For example, in the context of a IL2 proprotein of the disclosure, separate components (e.g., an Fc domain and an IL2Rα moiety) can be operably linked directly or through peptide linker sequences. In the context of a nucleic acid encoding a fusion protein, such as a half antibody of an IL2 proprotein of the disclosure, “operably linked” means that the two nucleic acids are joined such that the amino acid sequences encoded by the two nucleic acids remain in-frame. In the context of transcriptional regulation, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system.

Polypeptide, Peptide and Protein: The terms “polypeptide”, “peptide” and “protein” are used interchangeably herein to refer to a polymer of amino acid residues.

Proprotein: A “proprotein” is a protein precursor that is inactive and which can be activated by proteolysis by a protease. Thus, proproteins are “protease activatable”.

Protease: The term “protease” as used herein refers to any enzyme that that catalyzes hydrolysis of a peptide bond. Generally, the proteases useful in the present disclosure, e.g., the proteases described in Section 6.5.1, recognize and cleaves a specific sequence motif, e.g., a substrate as described in Section 6.5.2. Preferably, the proteases are expressed at higher levels in cancer tissues as compared to normal tissues.

Protease-cleavable linker: As used herein, the term “protease-cleavable linker” or “PCL” refers to a peptide whose amino acid sequence contains one or more (e.g., two, three or more) substrate sequences for one or more proteases. Exemplary protease-cleavable linkers are described in Section 6.5 and exemplary protease-cleavable linker sequences are disclosed in Section 6.5.4.

Recognize: The term “recognize” as used herein refers to an antibody or antibody fragment (e.g., a targeting moiety) that finds and interacts (e.g., binds) with its epitope.

Single Chain Fab or scFab: The term “single chain Fab” or “scFab” as used herein refers an ABD comprising a VH domain, a CH1 domain, a VL domain, a CL domain and a linker. In some embodiments, the foregoing domains and linker are arranged in one of the following orders in a N-terminal to C-terminal orientation: (a) VH-CH1-linker-VL-CL, (b) VL-CL-linker-VH-CH1, (c) VH-CL-linker-VL-CH1 or (d) VL-CH1-linker-VH-CL. Linkers are suitably noncleavable linkers of at least 30 amino acids, preferably between 32 and 50 amino acids. Single chain Fab fragments are typically stabilized via the natural disulfide bond between the CL domain and the CH1 domain. In addition, these single chain Fab molecules might be further stabilized by generation of interchain disulfide bonds via insertion of cysteine residues (e.g., at position 44 in the VH domain and position 100 in the VL domain according to Kabat numbering).

Single Chain Fv or scFv: The term “single-chain Fv” or “scFv” as used herein refers to ABDs comprising the VH and VL domains of an antibody, wherein these domains are present in a single polypeptide chain. Preferably, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds. (1994), Springer-Verlag, New York, pp. 269-315. The VH and VL and be arranged in the N- to C-terminal order VH-VL or VL-VH, typically separated by a linkers, for example a linker as set forth in Table E.

Spacer: As used herein, the term “spacer” refers to a peptide, the amino acid sequence of which is not a substrate for a protease, incorporated into a linker containing a substrate. A spacer can be used to separate the substrate from other domains in a molecule, for example an ABD. In some aspects, residues in the spacer minimize aminopeptidase and/or exopeptidase action to prevent cleavage of N-terminal amino acids.

Specifically (or selectively) binds: The term “specifically (or selectively) binds” to an antigen or an epitope refers to a binding reaction that is determinative of the presence of a cognate antigen or an epitope in a heterogeneous population of proteins and other molecules. The binding reaction can be but need not be mediated by an antibody or antibody fragment. The term “specifically binds” does not exclude cross-species reactivity. For example, an antigen-binding domain (e.g., an antigen-binding fragment of an antibody) that “specifically binds” to an antigen from one species may also “specifically bind” to that antigen in one or more other species. Thus, such cross-species reactivity does not itself alter the classification of an antigen-binding domain as a “specific” binder. In certain embodiments, an antigen-binding domain of the disclosure that specifically binds to a human antigen has cross-species reactivity with one or more non-human mammalian species, e.g., a primate species (including but not limited to one or more of Macaca fascicularis, Macaca mulatta, and Macaca nemestrina) or a rodent species, e.g., Mus musculus.

Subject: The term “subject” includes human and non-human animals. Non-human animals include all vertebrates, e.g., mammals and non-mammals, such as non-human primates, sheep, dog, cow, chickens, amphibians, and reptiles. In preferred embodiments, the subject is human.

Substrate: The term “substrate” refers to peptide sequence on which a protease will act and within which the protease will cleave a peptide bond.

Target Molecule: The term “target molecule” as used herein refers to any biological molecule (e.g., protein, carbohydrate, lipid or combination thereof) expressed on a cell surface or in the extracellular matrix that can be specifically bound by a targeting moiety in an IL2 proprotein of the disclosure.

Targeting Moiety: The term “targeting moiety” as used herein refers to any molecule or binding portion (e.g., an immunoglobulin or an antigen binding fragment) thereof that can bind to a cell surface or extracellular matrix molecule at a site to which an IL2 proprotein of the disclosure is to be localized, for example on tumor cells or on lymphocytes in the tumor microenvironment. In some embodiments, the targeting moiety binds to a TAA. In other embodiments, the targeting moiety binds to a TCA. The targeting moiety can also have a functional activity in addition to localizing an IL2 proprotein to a particular site. For example, a targeting moiety that binds to a checkpoint inhibitor such as PD1 can also exhibit anti-tumor activity or enhance the anti-tumor activity by IL2, for example by inhibiting PD1 signaling.

T-Cell Antigen, TCA: The term “T-cell antigen” or “TCA” refers to a molecule (typically a protein, carbohydrate, lipid or some combination thereof) that is expressed on the surface of a T-lymphocyte and is useful for the preferential targeting of a pharmacological agent to a particular site. In some embodiments, the site is cancer tissue and/or the T-cell antigen is a tumor reactive lymphocyte antigen, a cell surface molecule of tumor or viral lymphocytes, or a checkpoint inhibitor expressed on a T-lymphocyte.

Tumor: The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

Tumor-Associated Antigen, TAA: The term “tumor-associated antigen” or “TAA” refers to a molecule (typically a protein, carbohydrate, lipid or some combination thereof) that is expressed on the surface of a cancer cell, either entirely or as a fragment (e.g., MHC/peptide), and which is useful for the preferential targeting of a pharmacological agent to the cancer cell. In some embodiments, a TAA is a marker expressed by both normal cells and cancer cells, e.g., a lineage marker. In some embodiments, a TAA is a cell surface molecule that is overexpressed in a cancer cell in comparison to a normal cell, for instance, 1-fold over expression, 2-fold overexpression, 3-fold overexpression or more in comparison to a normal cell. In some embodiments, a TAA is a cell surface molecule that is inappropriately synthesized in the cancer cell, for instance, a molecule that contains deletions, additions or mutations in comparison to the molecule expressed on a normal cell. In some embodiments, a TAA will be expressed exclusively on the cell surface of a cancer cell, entirely or as a fragment (e.g., MHC/peptide), and not synthesized or expressed on the surface of a normal cell. Accordingly, the term “TAA” encompasses antigens that are specific to cancer cells, sometimes known in the art as tumor-specific antigens (“TSAs”).

Treat, Treatment, Treating: As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more IL2 proproteins of the disclosure. In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

Universal Light Chain, ULC: The term “universal light chain” or “ULC” as used herein refers to a light chain variable region (VL) that can pair with more than on heavy chain variable region (VL). In the context of a targeting moiety, the term “universal light chain” or “ULC” refers to a light chain polypeptide capable of pairing with the heavy chain region of the targeting moiety and also capable of pairing with other heavy chain regions. ULCs can also include constant domains, e.g., a CL domain of an antibody. Universal light chains are also known as “common light chains.

VH: The term “VH” refers to the variable region of an immunoglobulin heavy chain of an antibody, including the heavy chain of an Fv, scFv, dsFv or Fab.

VL: The term “VL” refers to the variable region of an immunoglobulin light chain, including the light chain of an Fv, scFv, dsFv or Fab.

### 6.2. IL2 Proproteins

The present disclosure relates to IL2 proproteins comprising an IL2 moiety, an IL2Rα moiety, and a protease-cleavable linker, arranged so that the IL2Rα diminishes or blocks the activity of the IL2 moiety. The IL2 proprotein is configured such that upon encountering a protease, e.g., a protease that is overexpressed in the tumor environment, the protease-cleavable linker is cleaved and IL2 is released and stimulates cytotoxic T-cell activity against tumor cells. Typically, the IL2 proproteins of the disclosure are dimeric and comprise two Fc domains that associate for form an Fc region, C-terminal to which the IL2Rα moiety, the protease-cleavable linker and the IL2 moiety are arranged in N- to C-terminal order. The IL2 proproteins may further comprise a linker, preferably a non-cleavable linker, between the Fc domain and the IL2Rα moiety.

Accordingly, the IL2 proproteins of the disclosure generally comprise: (a) a first Fc domain and a second Fc domain capable of associating to form an Fc region; (b) two IL2Rα moieties C-terminal to the Fc domains optionally preceded by linkers, preferably non-cleavable linkers; (c) two protease-cleavable linkers C-terminal to the IL2Rα moieties; and (d) two IL2 moieties C-terminal to the protease-cleavable linkers. The IL2 moiety in the IL2 proprotein is in an inactive form by virtue of masking by the IL2Rα moiety, but is released following protease-cleavage of the protease-cleavable linker, e.g., in the tumor environment. Examples of suitable IL2 moieties are described in Section 6.3, examples of suitable IL2Rα moieties are described in Section 6.4, and examples of suitable protease-cleavable linkers are described in Section 6.5.

Generally, the IL2 proproteins of the disclosure contain multiple linkers. Preferably, other than the protease-cleavable linker whose cleavage releases the IL2 moiety, the remaining linkers are non-cleavable. Examples of non-cleavable linkers are set forth in Section 6.6.

The IL2 proproteins may further comprise one or more targeting moieties, and in some embodiments comprise two targeting moieties N-terminal to the Fc domains. Examples of targeting moieties are described in 6.7 and suitable targeting moiety formats are described in Section 6.8. In some embodiments, the Fc domains comprise hinge domains at their N-termini. Suitable Fc domains with or without hinge sequences are described in Section 6.9.

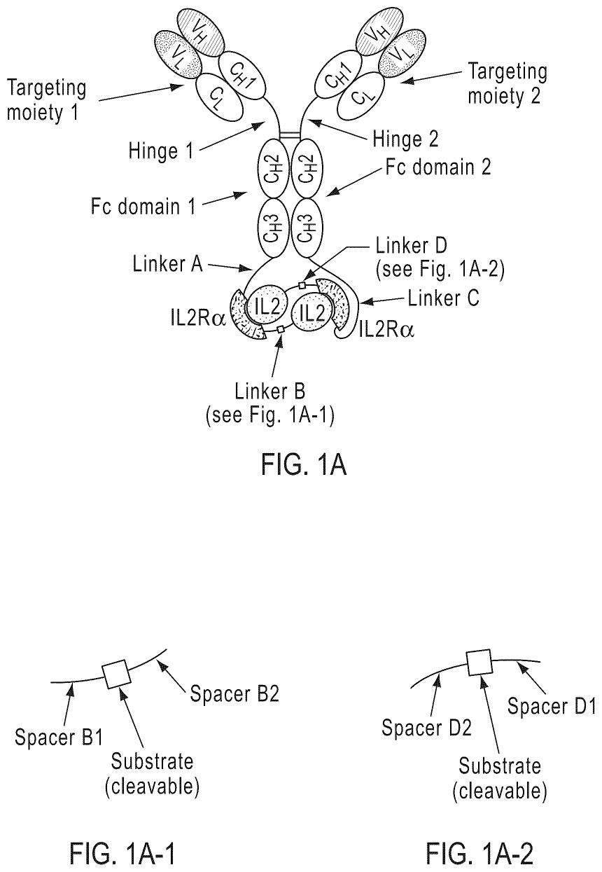

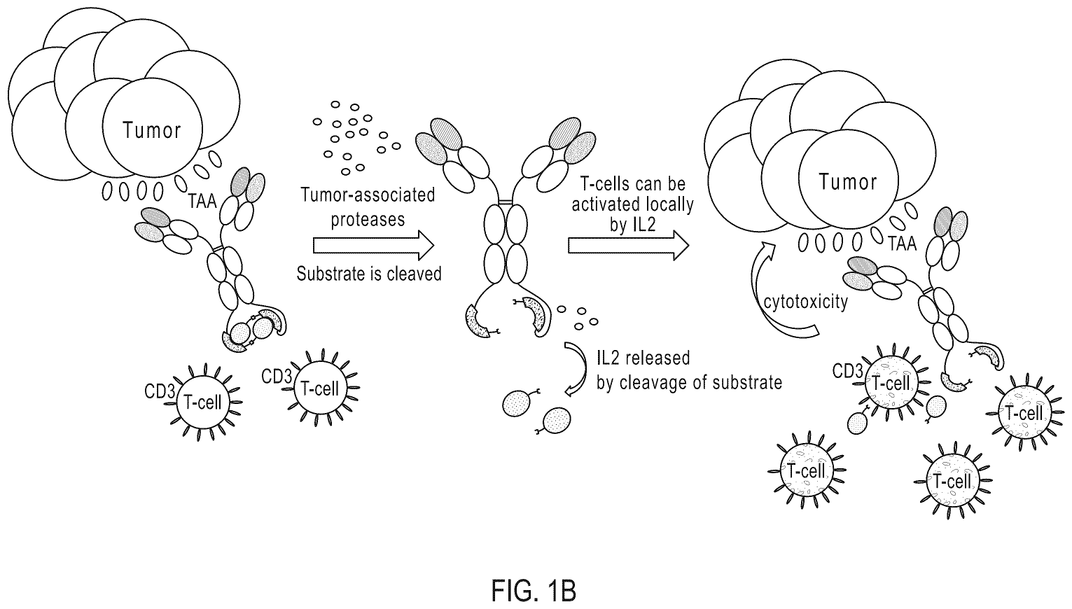

An exemplary IL2 proprotein is depicted in FIG. 1A. The IL2 proprotein comprises:
a) a first polypeptide chain comprising the heavy chain of a Fab associated with the light chain of a Fab on a separate polypeptide chain, together forming a first targeting moiety, followed by an Fc domain comprising a hinge domain, followed by a linker (“Linker A”) which is preferably a non-cleavable linker, followed by an IL2Rα moiety, followed by a protease-cleavable linker (“Linker B”), followed by an IL2 moiety; and
b) a second polypeptide chain comprising the heavy chain of a Fab associated with the light chain of a Fab on a separate polypeptide chain, together forming a second targeting moiety, followed by an Fc domain comprising a hinge domain, followed by a linker (“Linker C”) which is preferably a non-cleavable linker, followed by an IL2Rα moiety, followed by a protease-cleavable linker (“Linker D”), followed by an IL2 moiety.

6.3. The IL2 Moiety

The IL2 moiety of the IL2 proproteins of the disclosure comprises a wild type or variant IL2 moiety.

In eukaryotic cells human IL2 is synthesized as a precursor polypeptide of 153 amino acids, from which 20 amino acids are removed to generate mature secreted IL2 (Taniguchi et al., 1983, Nature 302(5906):305-10). Mature human IL2 has the following amino acid sequence:

(SEQ ID NO: 2)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT

In some embodiments, the IL2 moieties of the disclosure are not CD122 directed, e.g., they do not have amino acid substitutions in the IL2 moiety that make them preferentially bind to IL2RP as compared to IL2Rα.

In some embodiments, the IL2 moieties of the disclosure are CD25 directed, e.g., they have one or more amino acid substitutions in the IL2 moiety that make them preferentially bind to IL2Rα as compared to IL2Rβ.

In certain embodiments, the IL2 proproteins of the disclosure have one or more amino acid substitutions in the IL2 moiety that reduce binding to IL2Rβ. For example, in some embodiments, the IL2 moiety can have up to 50-fold (and in some embodiments up to 100-fold) to 1,000-fold attenuated binding to human IL2RP as compared to wild-type human IL2.

The IL2 moiety with reduced binding to IL2RP can retain its affinity to IL2Rα, or have reduced binding to IL2Rα. For example, in some embodiments, the IL2 moiety can have up to 50-fold attenuated binding to human ffα as compared to wild-type human IL2.

Other characteristics of useful IL2 variants may include the ability to induce proliferation of IL2Rα-bearing CD8+ T cells in tumors, the ability to induce IL2 signaling in IL2Rα-bearing CD8+ T cells in tumors, and an improved therapeutic index.

In one embodiment, the IL2 moiety comprises one or more amino acid substitutions that reduce affinity to IL2RP and preserve affinity to IL2Rα. An exemplary amino acid substitution is N88D. Other amino acid substitutions that reduce or abolish the affinity of IL2 to IL2RP are D20T, N88R, N88D or Q126D (see e.g., US Patent Publication No. US 2007/0036752).

In one embodiment, the IL2 moiety comprises one or more amino acid substitutions that reduce affinity to IL2Rα and preserve, or reduces affinity to a lesser degree, to IL2RP, resulting in CD122 directed IL2 moieties. Exemplary CD122 directed IL2 moieties are those comprising both H16A and F42A substitutions. Accordingly, in some embodiments, the IL2 moiety comprises the amino acid sequence of human IL2 with H16A and F42A substitutions, as shown below:

(SEQ ID NO: 124)
SAPTSSSTKKTQLQLEALLLDLQMILNGINNYKNPKLTRMLTAKFYMPKK
ATELKHLQCLEEELKPLEEVLNLAQSKNFHLRPRDLISNINVIVLELKGS
ETTFMCEYADETATIVEFLNRWITFCQSIISTLT

In certain embodiments, the IL2 moiety comprises an amino acid substitution which eliminates the O-glycosylation site of IL2 at a position corresponding to residue 3 of human IL2. Exemplary amino acid substitutions at T3 are T3A, T3G, T3Q, T3E, T3N, T3D, T3R, T3K, and T3P. In a specific embodiment, the substitution is T3A.

The IL2 moiety is preferably essentially a full-length IL2 molecule, e.g., a human IL2 molecule. In certain embodiments the IL2 moiety is a human IL-2 molecule.

C125 can be substituted with S, V, or A to reduce protein aggregation, as described in U.S. Pat. No. 4,518,584.

As described therein, one may also delete the N-terminal alanine residue of IL2, resulting in des-A1 IL2.

Further, the IL2 moiety may include a substitution of methionine 104 with a neutral amino acid such as alanine, as described in U.S. Pat. No. 5,206,344.

Accordingly, the IL2 moieties of the disclosure can have amino acid deletions and/or substitutions selected from des-A1 M104A IL2, des-A1 M104A C125S IL2, M104A IL2, M104A C125A IL2, des-A1 M104A C125A IL2, or M104A C125S IL2, in addition to other variations alter the binding of IL2 to its receptor. These and other mutants may be found in U.S. Pat. No. 5,116,943 and in Weiger et al., 1989, Eur J Biochem 180:295-300.

In various aspects, any of the foregoing IL2 moieties comprises an amino acid sequence having at least about 90%, at least about 91%, at least about 92%, about at least 93%, at least about 94%, at eat least about 95%, at least about 96%, at least about 97%, at least about 98%, at least about 99% or 100% sequence identity to mature human IL2.

### 6.4. The IL2Rα Moiety

The IL2 proproteins of the disclosure comprise an IL2Rα moiety, comprising or consisting of an IL2-binding domain of IL2Rα, e.g., the extracellular domain of an IL2Rα. The sequence of the mature human IL2Rα extracellular domain (corresponding to amino acids 22-272 of human IL2Rα) is:

(SEQ ID NO: 3)

Glu Leu Cys Asp Asp Asp Pro Pro Glu Ile Pro His
Ala Thr Phe Lys Ala Met Ala Tyr Lys Glu Gly Thr
Met Leu Asn Cys Glu Cys Lys Arg Gly Phe Arg Arg
Ile Lys Ser Gly Ser Leu Tyr Met Leu Cys Thr Gly
Asn Ser Ser His Ser Ser Trp Asp Asn Gln Cys Gln
Cys Thr Ser Ser Ala Thr Arg Asn Thr Thr Lys Gln
Val Thr Pro Gln Pro Glu Glu Gln Lys Glu Arg Lys
Thr Thr Glu Met Gln Ser Pro Met Gln Pro Val Asp
Gln Ala Ser Leu Pro Gly His Cys Arg Glu Pro Pro
Pro Trp Glu Asn Glu Ala Thr Glu Arg Ile Tyr His
Phe Val Val Gly Gln Met Val Tyr Tyr Gln Cys Val
Gln Gly Tyr Arg Ala Leu His Arg Gly Pro Ala Glu
Ser Val Cys Lys Met Thr His Gly Lys Thr Arg Trp
Thr Gln Pro Gln Leu Ile Cys Thr Gly Glu Met Glu
Thr Ser Gln Phe Pro Gly Glu Glu Lys Pro Gln Ala
Ser Pro Glu Gly Arg Pro Glu Ser Glu Thr Ser Cys
Leu Val Thr Thr Thr Asp Phe Gln Ile Gln Thr Glu
Met Ala Ala Thr Met Glu Thr Ser Ile Phe Thr Thr
Glu Tyr Gln Val Ala Val Ala Gly Cys Val Phe Leu
Leu Ile Ser Val Leu Leu Leu Ser Gly Leu Thr Trp
Gln Arg Arg Gln Arg Lys Ser Arg Arg Thr Ile

The sequence of an IL2 binding portion of the human IL2Rα extracellular domain (comprising the two “sushi” domains, which corresponds to amino acids 22-186 of human IL2Rα) is:

(SEQ ID NO: 4)

Glu Leu Cys Asp Asp Asp Pro Pro Glu Ile Pro His
Ala Thr Phe Lys Ala Met Ala Tyr Lys Glu Gly Thr
Met Leu Asn Cys Glu Cys Lys Arg Gly Phe Arg Arg
Ile Lys Ser Gly Ser Leu Tyr Met Leu Cys Thr Gly
Asn Ser Ser His Ser Ser Trp Asp Asn Gln Cys Gln
Cys Thr Ser Ser Ala Thr Arg Asn Thr Thr Lys Gln
Val Thr Pro Gln Pro Glu Glu Gln Lys Glu Arg Lys
Thr Thr Glu Met Gln Ser Pro Met Gln Pro Val Asp
Gln Ala Ser Leu Pro Gly His Cys Arg Glu Pro Pro
Pro Trp Glu Asn Glu Ala Thr Glu Arg Ile Tyr His
Phe Val Val Gly Gln Met Val Tyr Tyr Gln Cys Val
Gln Gly Tyr Arg Ala Leu His Arg Gly Pro Ala Glu
Ser Val Cys Lys Met Thr His Gly Lys Thr Arg Trp
Thr Gln Pro Gln Leu Ile Cys Thr Gly

The sequence of an alternative IL2 binding portion of the human IL2Rα extracellular domain, which corresponds to amino acids 22-240 of human IL2Rα, is:

(SEQ ID NO: 5)

Glu Leu Cys Asp Asp Asp Pro Pro Glu Ile Pro His
Ala Thr Phe Lys Ala Met Ala Tyr Lys Glu Gly Thr
Met Leu Asn Cys Glu Cys Lys Arg Gly Phe Arg Arg
Ile Lys Ser Gly Ser Leu Tyr Met Leu Cys Thr Gly
Asn Ser Ser His Ser Ser Trp Asp Asn Gln Cys Gln
Cys Thr Ser Ser Ala Thr Arg Asn Thr Thr Lys Gln
Val Thr Pro Gln Pro Glu Glu Gln Lys Glu Arg Lys
Thr Thr Glu Met Gln Ser Pro Met Gln Pro Val Asp
Gln Ala Ser Leu Pro Gly His Cys Arg Glu Pro Pro
Pro Trp Glu Asn Glu Ala Thr Glu Arg Ile Tyr His
Phe Val Val Gly Gln Met Val Tyr Tyr Gln Cys Val
Gln Gly Tyr Arg Ala Leu His Arg Gly Pro Ala Glu
Ser Val Cys Lys Met Thr His Gly Lys Thr Arg Trp
Thr Gln Pro Gln Leu Ile Cys Thr Gly Glu Met Glu
Thr Ser Gln Phe Pro Gly Glu Glu Lys Pro Gln Ala
Ser Pro Glu Gly Arg Pro Glu Ser Glu Thr Ser Cys
Leu Val Thr Thr Thr Asp Phe Gln Ile Gln Thr Glu
Met Ala Ala Thr Met Glu Thr Ser Ile Phe Thr Thr
Glu Tyr Gln

The IL2Rα moiety preferably comprises an amino acid sequence with at least about 90%, at least about 95%, at least about 96%, at least about 97%, at least about 98%, at least about 99% or 100% sequence identity to any of the sequences above, i.e., any one of amino acids 22-186 of IL2Rα, amino acids 22-240 of IL2Rα, or amino acids 22-272 of IL2Rα, or any IL2 binding portion thereof.

In certain aspects, the IL2Rα moiety can comprise or consist of an amino acid sequence having at least about 90%, at least about 95%, at least about 96%, at least about 97%, at least about 98%, at least about 99% or 100% sequence identity to an IL2 binding portion of human IL2Rα, optionally wherein the binding portion has an amino acid sequence of (a) at least 160 amino acids, at least 161 amino acids, at least 162 amino acids, at least 164 amino acids or at least 165 amino acids and/or (b) up to 251, up to 240, up to 230, up to 220, up to 210, up to 200, up to 190, up to 180 or up to 170 amino acids of the extracellular domain of human IL2Rα. In particular embodiments, the portion of human IL2Rα is bounded by any one of (a) and (b) in the preceding sentence, e.g., at least 160 and up to 180 amino acids from human IL2Rα, at least 162 and up to 200 amino acids from human IL2Rα, at least 160 and up to 220 amino acids from human IL2Rα, at least 164 and up to 190 amino acids from human IL2Rα, and so on and so forth.

In some embodiments, the IL2Rα moiety comprises or consists of an amino acid sequence having at least about 90%, at least about 95%, at least about 96%, at least about 97%, at least about 98%, at least about 99% or 100% sequence identity to amino acids 22-186, with or without an additional up to 5 amino acids, up to 10 amino acids, up to 15 amino acids, up to 20 amino acids, up to 30 amino acids, or up to 40 amino acids C-terminal to amino acid residue 186, of IL2Rα.

In certain embodiments, the IL2Rα moiety has at least one fewer O-glycosylation and/or N-glycosylation compared to the extracellular domain of native IL2Rα, for example by a substitution at one or more of amino acid N49, amino acid N68, amino acid T74, amino acid T85, amino acid T197, amino acid T203, amino acid T208, and amino acid T216. In some embodiments, the one or more substitutions are from asparagine to an amino acid selected from the group consisting of alanine, threonine, serine, arginine, aspartic acid, glutamine, glutamic acid, glycine, histidine, isoleucine, leucine, lysine, methionine, phenylalanine, proline, tryptophan, tyrosine, and valine. In some embodiments, the one or more substitutions are from threonine to an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, tryptophan, tyrosine, and valine. In some embodiments, the one or more substitutions are at amino acid S50 (e.g., S50P), amino acid S51 (e.g., S51R, S51N, S51D, S51C, S51Q, S51E, S51G, S51H, S51I, S51L, S51K, S51M, S51F, S51P, S51W, S51Y, or S51V), amino acid T69 (e.g., T69P), amino acid T70 (e.g., T70R, T70N, T70D, T70C, T70Q, T70E, T70G, T70H, T70I, T70L, T70K, T70M, T70F, T70P, T70W, T70Y, or T70V, amino acid C192 (e.g., C192R, C192N, C192D, C192Q, C192E, C192G, C192H, C192I, C192L, C192K, C192M, C192F, C192P, C192W, C192Y, or C192V), or any combination thereof.

### 6.5. Protease-Cleavable Linkers

The IL2 proproteins of the disclosure comprise a linker with one or more substrates for a protease and whose cleavage results in activation IL2, typically located between the IL2Rα moiety and the IL2 moiety.

A protease-cleavable linker can range from 20 amino acids to 80 or more amino acids, and in certain aspects a non-cleavable peptide linker ranges from 20 amino acids to 60 amino acids, 20 amino acids to 40 amino acids, from 30 amino acids to 50 amino acids, from 20 amino acids to 80 amino acids, or from 30 amino acids to 70 amino acids in length.

The protease-cleavable linkers comprise one or more substrate sequences for one or more proteases, for example one or more of the proteases set forth in Section 6.5.1. The one or more substrate sequences, e.g., one or more of the substrate sequences set forth in Section 6.5.2, are typically flanked by one or more spacer sequences, e.g., spacer sequences as described in Section 6.5.3. Each protease-cleavable linker can include one, two, three or more substrate sequences. The spacer sequences can be adjoining, overlapping, or separated by spacer sequences. Preferably, the C- and N-termini of the protease-cleavable linkers contain spacer sequences.

Exemplary protease-cleavable linker sequences ae set forth in Section 6.5.4

### 6.5.1. Proteases

Exemplary protease whose substrate sequences can be incorporated into the protease-cleavable linkers are set forth in Table A below.

TABLE A
Exemplary Proteases for Substrate Cleavage

| | | |
|---|---|---|
| ADAMS, ADAMTS, e.g. | Caspases, e.g., | MMP24 |
| ADAM8 | Caspase 1 | MMP26 |
| ADAM9 | Caspase 2 | MMP27 |
| ADAM10 | Caspase 3 | |
| ADAM12 | Caspase 4 | |
| ADAM15 | Caspase 5 | |
| ADAM17/TACE | Caspase 6 | |
| ADAMDEC1 | Caspase 7 | |
| ADAMTS1 | Caspase 8 | Cysteine proteinases, e.g., |
| ADAMTS4 | Caspase 9 | Cruzipain |
| ADAMTS5 | Caspase 10 | Legumain |
| | Caspase 14 | Otubain-2 |
| Aspartate proteases, e.g., | | |
| BACE | Cysteine cathepsins, e.g., | KLKs, e.g., |
| Renin | Cathepsin B | KLK4 |
| | Cathepsin C | KLK5 |
| Aspartic cathepsins, e.g., | Cathepsin K | KLK6 |
| Cathepsin D | Cathepsin L | KLK7 |
| Cathepsin E | Cathepsin S | KLK8 |
| | Cathepsin V/L2 | KLK10 |
| NS3/4A | Cathepsin X/Z/P | KLK11 |
| PACE4 | | KLK13 |
| Plasmin | MMPs, e.g., | KLK14 |
| PSA | MMP1 | |
| tPA | MMP2 | Metallo proteinases, e.g., |
| Thrombin | MMP3 | Meprin |
| Tryptase | | Neprilysin |
| uPA | MMP7 | PSMA |
| | MMP8 | BMP-1 |
| Type II Transmembrane | MMP9 | |
| Serine Proteases (TTSPs), | MMP10 | |
| e.g., | | |
| DESC1 | MMP11 | Serine proteases, e.g., |
| DPP-4 | MMP12 | activated protein C |
| FAP | MMP13 | Cathepsin A |
| Hepsin | MMP14 | Cathepsin G |
| Matriptase-2 | MMP15 | Chymase |
| MT/SP1/Matriptase | MMP16 | coagulation factor proteases |
| | MMP17 | (e.g., FVIIa, FIXa, FXa, |
| | | FXIa, FXIIa) |
| TMPRSS2 | MMP19 | Human Neutrophil Elastase |
| TMPRSS3 | MMP20 | Lactoferrin |
| TMPRSS4 | MMP23 | |

In particular embodiments, the protease is matrix metalloprotease (MMP)-2, MMP-9, legumain asparaginyl endopeptidase, thrombin, fibroblast activation protease (FAP), MMP-1, MMP-3, MMP-7, MMP-8, MMP-12, MMP-13, MMP-14, membrane type 1 matrix metalloprotease (MT1-MMP), plasmin, transmembrane protease, serine (TMPRSS-3/4), cathepsin A, cathepsin B, cathepsin D, cathepsin E, cathepsin F, cathepsin H, cathepsin K, cathepsin L, cathepsin L2, cathepsin O, cathepsin S, caspase 1, caspase 2, caspase 3, caspase 4, caspase 5, caspase 6, caspase 7, caspase 8, caspase 9, caspase 10, caspase 11, caspase 12, caspase 13, caspase 14, human neutrophil elastase, urokinase/urokinase-type plasminogen activator (uPA), a disintegrin and metalloprotease (ADAM)10, ADAM12, ADAM17, ADAM with thrombospondin motifs (ADAMTS), ADAMTS5, beta secretase (BACE), granzyme A, granzyme B, guanidinobenzoatase, hepsin, matriptase, matriptase 2, meprin, neprilysin, prostate-specific membrane antigen (PSMA), tumor necrosis factor-converting enzyme (TACE), kallikrein-related peptidase (KLK)3, KLK5, KLK7, KLK11, NS3/4 protease of hepatitis C virus (HCV-NS3/4), tissue plasminogen activator (tPA), calpain, calpain 2, glutamate carboxypeptidase II, plasma kallikrein, AMSH-like protease, AMSH, γ-secretase component, antiplasmin cleaving enzyme (APCE), decysin 1, apoptosis-related cysteine peptidase, or N-acetylated alpha-linked acidic dipeptidase-like 1.

### 6.5.2. Substrates

Exemplary substrate sequences that are cleavable by a tumor protease and can be incorporated into the protease-cleavable linkers are set forth in Table B below.

TABLE B
Substrate Sequences for Protease-Cleavable Linkers

| Substrate Sequence | Designation | Cleaving Protease |
|---|---|---|
| (DE)₈RPLALWRS(DR)₈ (SEQ ID NO: 6) | SU1 | MMP7 |
| AARGPAIH (SEQ ID NO: 7) | SU2 | |
| AAYHLVSQ (SEQ ID NO: 8) | SU3 | Collagenase |
| AGLGISST (SEQ ID NO: 9) | SU4 | Collagenase |
| AGLGVVER (SEQ ID NO: 10) | SU5 | Collagenase |
| ALAL (SEQ ID NO: 11) | SU6 | Lysosomal Enzyme |
| ALFFSSPP (SEQ ID NO: 12) | SU7 | |
| ALFKSSFP (SEQ ID NO: 13) | SU8 | |
| ALLLALL (SEQ ID NO: 14) | SU9 | TOP |
| AQFVLTEG (SEQ ID NO: 15) | SU10 | Collagenase |
| AQNLLGMV (SEQ ID NO: 16) | SU11 | |
| AVGLLAPP (SEQ ID NO: 17) | SU12 | Serine protease |
| DAFK (SEQ ID NO: 18) | SU13 | Urokinase plasminogen |
| | | activator (uPA) |
| DEVD (SEQ ID NO: 19) | SU14 | Caspase-3 |
| DEVDP (SEQ ID NO: 20) | SU15 | Caspase-3 |
| DPRSFL (SEQ ID NO: 21) | SU16 | Thrombin |
| DVAQFVLT (SEQ ID NO: 22) | SU17 | Collagenase |
| DVLK (SEQ ID NO: 23) | SU18 | Plasmin |
| DWLYWPGI (SEQ ID NO: 24) | SU19 | |
| EDDDDKA (SEQ ID NO: 25) | SU20 | Enterokinase |
| EP(Cit)G(Hof)YL (SEQ ID NO: 26) | SU21 | MMP2, MMP9, MMP14 |
| EPQALAMS (SEQ ID NO: 27) | SU22 | Collagenase |
| ESLPVVAV (SEQ ID NO: 28) | SU23 | Collagenase |
| ESPAYYTA (SEQ ID NO: 29) | SU24 | MMP |
| F(Pip)RS | SU25 | Thrombin |
| FK | SU26 | Lysosomal Enzyme |
| FPRPLGITGL (SEQ ID NO: 30) | SU27 | |
| FRLLDWQW (SEQ ID NO: 31) | SU28 | |
| GFLG (SEQ ID NO: 32) | SU29 | Lysosomal Enzyme |
| GGAANLVRGG (SEQ ID NO: 33) | SU30 | MMP11 |
| GGGRR (SEQ ID NO: 34) | SU31 | Urokinase plasminogen |
| | | activator (uPA) |
| GGPRGLPG (SEQ ID NO: 35) | SU32 | Cathepsin K |
| GGQPSGMWGW (SEQ ID NO: 36) | SU33 | |
| GGSIDGR (SEQ ID NO: 37) | SU34 | Factor Xa |
| GGWHTGRN (SEQ ID NO: 38) | SU35 | |
| GIAGQ (SEQ ID NO: 39) | SU36 | Collagenase |
| GKAFRR (SEQ ID NO: 40) | SU37 | Kallikrein 2 |
| GPAGLYAQ (SEQ ID NO: 41) | SU38 | |
| GPAGMKGL (SEQ ID NO: 42) | SU39 | |
| GPEGLRVG (SEQ ID NO: 43) | SU40 | Collagenase |
| GPLGIAGI (SEQ ID NO: 44) | SU41 | Collagenase |
| GPLGVRG (SEQ ID NO: 45) | SU42 | |
| GPQGIAGQ (SEQ ID NO: 46) | SU43 | Collagenase |
| GPQGLLGA (SEQ ID NO: 47) | SU44 | Collagenase |
| GPRSFG (SEQ ID NO: 48) | SU45 | |
| GPRSFGL (SEQ ID NO: 49) | SU46 | |
| GPSHLVLT (SEQ ID NO: 50) | SU47 | |
| GVSQNYPIVG (SEQ ID NO: 51) | SU48 | HIV Protease |
| GVVQASCRLA (SEQ ID NO: 52) | SU49 | CMV Protease |
| GWEHDG (SEQ ID NO: 53) | SU50 | Interleukin 1β converting |
| | | enzyme |
| HSSKLQ (SEQ ID NO: 54) | SU51 | Prostate Specific Antigen |
| HSSKLQEDA (SEQ ID NO: 55) | SU52 | Prostate Specific Antigen |
| HSSKLQL (SEQ ID NO: 56) | SU53 | Prostate Specific Antigen |
| HTGRSGAL (SEQ ID NO: 57) | SU54 | |
| IDGR (SEQ ID NO: 58) | SU55 | Factor Xa |
| IEGR (SEQ ID NO: 59) | SU56 | Factor Xa |
| ILPRSPAF (SEQ ID NO: 60) | SU57 | |
| IPVSLRSG (SEQ ID NO: 61) | SU58 | MMP |
| ISSGL (SEQ ID NO: 62) | SU59 | MMP |
| ISSGLL (SEQ ID NO: 63) | SU60 | MMP |
| ISSGLLS (SEQ ID NO: 64) | SU61 | MMP |
| ISSGLLSS (SEQ ID NO: 65) | SU62 | MMP |
| ISSGLSS (SEQ ID NO: 66) | SU63 | MMP |
| KGSGDVEG (SEQ ID NO: 67) | SU64 | Caspase-3 |
| KQEQNPGST (SEQ ID NO: 68) | SU65 | FAP |
| KRALGLPG (SEQ ID NO: 69) | SU66 | MMP7 |
| LAAPLGLL (SEQ ID NO: 70) | SU67 | |
| LAPLGLQRR (SEQ ID NO: 71) | SU68 | |
| LAQKLKSS (SEQ ID NO: 72) | SU69 | |
| LAQRLRSS (SEQ ID NO: 73) | SU70 | |
| LEATA (SEQ ID NO: 74) | SU71 | MMP9 |
| LKAAPRWA (SEQ ID NO: 75) | SU72 | |
| LLAPSHRA (SEQ ID NO: 76) | SU73 | |
| LPGGLSPW (SEQ ID NO: 77) | SU74 | |
| LSGRSANI (SEQ ID NO: 78) | SU75 | Serine protease |
| LSGRSANP (SEQ ID NO: 79) | SU76 | Serine protease |
| LSGRSDDH (SEQ ID NO: 80) | SU77 | Serine protease |
| LSGRSDIH (SEQ ID NO: 81) | SU78 | Serine protease |
| LSGRSDNH (SEQ ID NO: 82) | SU79 | Serine protease |
| LSGRSDNI (SEQ ID NO: 83) | SU80 | Serine protease |
| LSGRSDNP (SEQ ID NO: 84) | SU81 | Serine protease |
| LSGRSDQG (SEQ ID NO: 85) | SU82 | Serine protease |
| LSGRSDQH (SEQ ID NO: 86) | SU83 | Serine protease |
| LSGRSDTH (SEQ ID NO: 87) | SU84 | Serine protease |
| LSGRSDYH (SEQ ID NO: 88) | SU85 | Serine protease |
| LSGRSGNH (SEQ ID NO: 89) | SU86 | Serine protease |
| LVLASSSFGY (SEQ ID NO: 90) | SU87 | Herpes Simplex Virus |
| | | Protease |
| MDAFLESS (SEQ ID NO: 91) | SU88 | Collagenase |
| MGLFSEAG (SEQ ID NO: 92) | SU89 | |
| MIAPVAYR (SEQ ID NO: 93) | SU90 | |
| MVLGRSLL (SEQ ID NO: 94) | SU91 | |
| NLL | SU92 | Cathepsin B |
| NTLSGRSENHSG (SEQ ID NO: 95) | SU93 | |
| NTLSGRSGNHGS (SEQ ID NO: 96) | SU94 | |
| PAGLWLDP (SEQ ID NO: 97) | SU95 | |
| PGGPAGIG (SEQ ID NO: 98) | SU96 | |
| PIC(Et)FF (SEQ ID NO: 99) | SU97 | Cathepsin D |
| PLGC(me)AG (SEQ ID NO: 100) | SU98 | MMP |
| PLGL (SEQ ID NO: 101) | SU99 | |
| PLGLAG (SEQ ID NO: 102) | SU100 | MMP |
| PLGLAX (SEQ ID NO: 103) | SU101 | MMP |
| PLGLWA (SEQ ID NO: 104) | SU102 | MMP |
| PLGLWSQ (SEQ ID NO: 105) | SU103 | MMP |
| PLTGRSGG (SEQ ID NO: 106) | SU104 | |
| PMAKK (SEQ ID NO: 107) | SU105 | |
| PPRSFL (SEQ ID NO: 108) | SU106 | Thrombin |
| PR(S/T)(L/I)(S/T) | SU107 | MMP9 |
| PRFRIIGG (SEQ ID NO: 109) | SU108 | Plasmin |
| PVGYTSSL (SEQ ID NO: 110) | SU109 | |
| PVQPIGPQ (SEQ ID NO: 111) | SU110 | Collagenase |
| QALAMSAI (SEQ ID NO: 112) | SU111 | Collagenase |
| QGRAITFI (SEQ ID NO: 113) | SU112 | |
| QNQALRMA (SEQ ID NO: 114) | SU113 | |
| RGPA (SEQ ID NO: 115) | SU114 | |
| RGPAFNPM (SEQ ID NO: 116) | SU115 | |
| RGPATPIM (SEQ ID NO: 117) | SU116 | |
| RKSSIIIRMRDVVL (SEQ ID NO: 118) | SU117 | Plasmin |
| RLQLKAC (SEQ ID NO: 119) | SU118 | MMP |
| RLQLKL (SEQ ID NO: 120) | SU119 | MMP |
| RMHLRSLG (SEQ ID NO: 121) | SU120 | |
| RPSPMWAY (SEQ ID NO: 122) | SU121 | |
| RQARVVNG (SEQ ID NO: 123) | SU122 | Matripase |
| SAGFSLPA (SEQ ID NO: 125) | SU123 | |
| SAPAVESE (SEQ ID NO: 126) | SU124 | Collagenase |
| SARGPSRW (SEQ ID NO: 127) | SU125 | |
| SGEPAYYTA (SEQ ID NO: 128) | SU126 | |
| SGGPLGVR (SEQ ID NO: 129) | SU127 | |
| SGRIGFLRTA (SEQ ID NO: 130) | SU128 | MMP14 |
| SGRSA (SEQ ID NO: 131) | SU129 | Urokinase plasminogen |
| | | activator (uPA) |
| SGRSANPRG (SEQ ID NO: 132) | SU130 | |
| SMLRSMPL (SEQ ID NO: 133) | SU131 | |
| SPLPLRVP (SEQ ID NO: 134) | SU132 | |
| SPLTGRSG (SEQ ID NO: 135) | SU133 | |
| SPRSIMLA (SEQ ID NO: 136) | SU134 | |
| SSRGPAYL (SEQ ID NO: 137) | SU135 | |
| SSRHRRALD (SEQ ID NO: 138) | SU136 | Plasmin |
| SSSFDKGKYKKGDDA (SEQ ID NO: 139) | SU137 | Plasmin |
| SSSFDKGKYKRGDDA (SEQ ID NO: 140) | SU138 | Plasmin |
| STFPFGMF (SEQ ID NO: 141) | SU139 | |
| TARGPSFK (SEQ ID NO: 142) | SU140 | |
| TGRGPSWV (SEQ ID NO: 143) | SU141 | |
| TSGRSANP (SEQ ID NO: 144) | SU142 | |
| TSTSGRSANPRG (SEQ ID NO: 145) | SU143 | |
| VAGRSMRP (SEQ ID NO: 146) | SU144 | |
| VAQFVLTE (SEQ ID NO: 147) | SU145 | Collagenase |
| VHMPLGFLGP (SEQ ID NO: 148) | SU146 | |
| VPLSLYSG (SEQ ID NO: 149) | SU147 | MMP9 |
| VVPEGRRS (SEQ ID NO: 150) | SU148 | |
| WATPRPMR (SEQ ID NO: 151) | SU149 | |
| YGAGLGVV (SEQ ID NO: 152) | SU150 | Collagenase |
| HPVGLLAR (SEQ ID NO: 261) | SU151 | |

### 6.5.3. Spacers

Exemplary spacer sequences that can be incorporated into the protease-cleavable linkers are set forth in Table C below. In addition to the spacer sequences set forth in Table C, any of the non-cleavable linker sequences described in Section 6.6, e.g., the non-cleavable linker sequences set forth in Table E, or portions thereof can be used as spacer sequences.

TABLE C
Spacer Sequences for Protease-
Cleavable Linkers

| Spacer Sequence | Designation |
|---|---|
| (GGGGS)ₙ (SEQ ID NO: 153) | SP1 |
| (GGGS)ₙ (SEQ ID NO: 154) | SP2 |
| (GGS)ₙ | SP3 |
| (GS)ₙ | SP4 |
| (GSGGS)ₙ (SEQ ID NO: 155) | SP5 |
| GGGGSGGGGS (SEQ ID NO: | SP6 |
| 156) | |
| GGGGSGGGGSGGGGS (SEQ | SP7 |
| ID NO: 157) | |
| GGGGSGGGGSGGGGSGGGGS | SP8 |
| (SEQ ID NO: 158) | |
| GGGKSGGGKSGGGKS (SEQ ID | SP9 |
| NO: 159) | |
| GGGKSGGKGSGKGGS (SEQ ID | SP10 |
| NO: 160) | |
| GGGS (SEQ ID NO: 161) | SP11 |
| GGGSG (SEQ ID NO: 162) | SP12 |
| GGKGSGGKGSGGKGS (SEQ ID | SP13 |
| NO: 163) | |
| GGSGGGGSGGGGS (SEQ ID | SP14 |
| NO: 164) | |
| GGSGGS (SEQ ID NO: 165) | SP15 |
| GGSGGSGGSGS (SEQ ID NO: | SP16 |
| 166) | |
| GSGGG (SEQ ID NO: 167) | SP17 |
| GSGSG (SEQ ID NO: 168) | SP18 |
| GSS | SP19 |
| GSSG (SEQ ID NO: 169) | SP20 |
| GSSGGSGGSG (SEQ ID NO: | SP21 |
| 170) | |
| GSSGGSGGSGG (SEQ ID NO: | SP22 |
| 171) | |
| GSSGGSGGSGGS (SEQ ID NO: | SP23 |
| 172) | |
| GSSGGSGGSGGSG (SEQ ID | SP24 |
| NO: 173) | |
| GSSGGSGGSGGSGGGS (SEQ | SP25 |
| ID NO: 174) | |
| GSSGGSGGSGS (SEQ ID NO: | SP26 |
| 175) | |
| GSSGT (SEQ ID NO: 176) | SP27 |
| GSSSG (SEQ ID NO: 177) | SP28 |
| QGQSGQ (SEQ ID NO: 178) | SP29 |
| QGQSGQG (SEQ ID NO: 179) | SP30 |
| QGQSGS (SEQ ID NO: 180) | SP31 |
| QSGQ (SEQ ID NO: 181) | SP32 |
| QSGQG (SEQ ID NO: 182) | SP33 |
| QSGS (SEQ ID NO: 183) | SP34 |
| SGQ | SP35 |
| SGQG (SEQ ID NO: 184) | SP36 |
| SGS | SP37 |
| (G)ₙ | SP38 |

In some embodiments, as used in Table C above, n is an integer from 1 to 10, e.g., 1, 2, 3, 4, 5, 6, 7, 8, 9 or 10.

### 6.5.4. Exemplary Protease-Cleavable Linkers

Exemplary protease-cleavable linkers comprising one or more substrate sequences as well as spacer sequences are set forth in Table 0 below.

TABLE D
Protease-Cleavable Linker Sequences

| Linker Sequence | Designation | Cleaving Protease(s) |
|---|---|---|
| GGGISSGLLSGRSDNHGGGISSG | PCL1 | |
| LLSGRSDNHGGS (SEQ ID NO: | | |
| 185) | | |
| GGGISSGLLSGRSDNHGGGISSG | PCL2 | |
| LLSGRSDNHGGSGGGISSGLLSG | | |
| RSDNHGGGISSGLLSGRSDNHGG | | |
| S (SEQ ID NO: 186) | | |
| GGSGGSIPVSLRSGGGISSGLLS | PCL3 | |
| GRSDNHGGSGGS (SEQ ID NO: | | |
| 187) | | |
| GGSGGSVPLSLYSGGGISSGLLS | PCL4 | |
| GRSDNHGGSGGS (SEQ ID NO: | | |
| 188) | | |
| GGSHPVGLLARGGGHPVGLLARG | PCL5 | |
| GGHPVGLLARGS (SEQ ID | | |
| NO: 189) | | |
| GGSHPVGLLARGGGHPVGLLARG | PCL6 | |
| GSGRSAGGSGRSA (SEQ ID | | |
| NO: 190) | | |
| AVGLLAPPGGLSGRSANI (SEQ | PCL7 | ADAM17_2, FAPa_1, CTSL1_1 |
| ID NO: 191) | | |
| AVGLLAPPGGLSGRSANP (SEQ | PCL8 | FAPa_1, ADAM17_2, CTSL1_1 |
| ID NO: 192) | | |
| AVGLLAPPGGLSGRSDDH (SEQ | PCL9 | MMP14_1, MMP14_1, MMP14_1 |
| ID NO: 193) | | |
| AVGLLAPPGGLSGRSDIH (SEQ | PCL10 | MMP14_1, MMP14_1, MMP14_1 |
| ID NO: 194) | | |
| AVGLLAPPGGLSGRSDNH (SEQ | PCL11 | MMP14_1, MMP14_1 |
| ID NO: 195) | | |
| AVGLLAPPGGLSGRSDNI (SEQ | PCL12 | MMP14_1, CTSL1_1, ADAM17_2 |
| ID NO: 196) | | |
| AVGLLAPPGGLSGRSDNP (SEQ | PCL13 | CTSL1_1, ADAM17_2, FAPa_1 |
| ID NO: 197) | | |
| AVGLLAPPGGLSGRSDQH (SEQ | PCL14 | |
| ID NO: 198) | | |
| AVGLLAPPGGLSGRSDTH (SEQ | PCL15 | FAPa_1, CTSL1_1, ADAM17_2 |
| ID NO: 199) | | |
| AVGLLAPPGGLSGRSDYH (SEQ | PCL16 | |
| ID NO: 200) | | |
| AVGLLAPPGGTSTSGRSANPRG | PCL17 | |
| (SEQ ID NO: 201) | | |
| AVGLLAPPSGRSANPRG (SEQ ID | PCL18 | |
| NO: 202) | | |
| AVGLLAPPTSGRSANPRG (SEQ | PCL19 | |
| ID NO: 203) | | |
| GGALFKSSFPGPAGLYAQPLAQK | PCL20 | CTSL1_1, MMP14_1, ADAM17_2 |
| LKSSGGK (SEQ ID NO: 204) | | |
| GGGGSGGGGSGGGGSFVGGTGGG | PCL21 | |
| GSGGGGSGGS (SEQ ID NO: | | |
| 205) | | |
| GGGGSGGGGSGGGGSISSGLLSG | PCL22 | |
| RSDNHGGSGGS (SEQ ID NO: | | |
| 206) | | |
| GGGGSGGGGSGGGGSVPLSLYSG | PCL23 | |
| GGSGGSGGSGS (SEQ ID NO: | | |
| 207) | | |
| GGGGSGGGGSGPLGLWSQGGGGS | PCL24 | |
| GGGGSGGGGSGG (SEQ ID | | |
| NO: 208) | | |
| GGGGSGGGGSKKAAPGGGGSGGG | PCL25 | |
| GSGGGGSGGS (SEQ ID NO: | | |
| 209) | | |
| GGGGSGGGGSKKAAPVNGGGGGS | PCL26 | |
| GGGGSGGGGS (SEQ ID NO: | | |
| 210) | | |
| GGGGSGGGGSPMAKKGGGGSGGG | PCL27 | |
| GSGGGGSGGS (SEQ ID NO: | | |
| 211) | | |
| GGGGSGGGGSPMAKKVNGGGGGS | PCL28 | |
| GGGGSGGGGS (SEQ ID NO: | | |
| 212) | | |
| GGGGSGGGGSQARAKGGGGSGGG | PCL29 | |
| GSGGGGSGGS (SEQ ID NO: | | |
| 213) | | |
| GGGGSGGGGSQARAKVNGGGGGS | PCL30 | |
| GGGGSGGGGS (SEQ ID NO: | | |
| 214) | | |
| GGGGSGGGGSRQARVVNGGGGGS | PCL31 | |
| GGGGSGGGGS (SEQ ID NO: | | |
| 215) | | |
| GGGGSGGGGSRQARVVNGGGGGS | PCL32 | |
| VPLSLYSGGGGGSGGGGS | | |
| (SEQ ID NO: 216) | | |
| GGGGSGGGGSRQARVVNSVPLSL | PCL33 | |
| YSGGGGGSGGGGS (SEQ ID | | |
| NO: 217) | | |
| GGGGSGGGGSVHMPLGFLGPGGG | PCL34 | |
| GSGGGGSGGS (SEQ ID NO: | | |
| 218) | | |
| GGGGSVHMPLGFLGPGRSRGSFP | PCL35 | |
| GGGGS (SEQ ID NO: 219) | | |
| GGGGSVHMPLGFLGPPMAKKGGG | PCL36 | |
| GSGGGGSGGS (SEQ ID NO: | | |
| 220) | | |
| GGGGSVHMPLGFLGPRQARVVNG | PCL37 | |
| GGGSGGGGS (SEQ ID NO: | | |
| 221) | | |
| GGGGSVHMPLGFLGPRQARVVNG | PCL38 | |
| GGGSGGGGSGG (SEQ ID NO: | | |
| 222) | | |
| GGPLAQKLKSSALFKSSFPGPAG | PCL39 | ADAM17_2, CTSL1_1, MMP14_1 |
| LYAQGGR (SEQ ID NO: 223) | | |
| GLSGRSDNHGGAVGLLAPP (SEQ | PCL40 | |
| ID NO: 224) | | |
| GLSGRSDNHGGVHMPLGFLGP | PCL41 | |
| (SEQ ID NO: 225) | | |
| ISSGLLSGRSANI (SEQ ID NO: | PCL42 | MMP, Serine protease |
| 226) | | |
| ISSGLLSGRSANP (SEQ ID NO: | PCL43 | MMP, Serine protease |
| 227) | | |
| ISSGLLSGRSANPRG (SEQ ID | PCL44 | MMP, Serine protease |
| NO: 228) | | |
| ISSGLLSGRSDDH (SEQ ID NO: | PCL45 | MMP, Serine protease |
| 229) | | |
| ISSGLLSGRSDIH (SEQ ID NO: | PCL46 | MMP, Serine protease |
| 230) | | |
| ISSGLLSGRSDNH (SEQ ID NO: | PCL47 | MMP, Serine protease |
| 231) | | |
| ISSGLLSGRSDNI (SEQ ID NO: | PCL48 | CTSL1_1, MMP14_1 |
| 232) | | |
| ISSGLLSGRSDNP (SEQ ID NO: | PCL49 | MMP, Serine protease |
| 233) | | |
| ISSGLLSGRSDQH (SEQ ID NO: | PCL50 | MMP, Serine protease |
| 234) | | |
| ISSGLLSGRSDTH (SEQ ID NO: | PCL51 | MMP, Serine protease |
| 235) | | |
| ISSGLLSGRSDYH (SEQ ID NO: | PCL52 | MMP, Serine protease |
| 236) | | |
| ISSGLLSGRSGNH (SEQ ID NO: | PCL53 | MMP, Serine protease |
| 237) | | |
| ISSGLLSSGGSGGSLSGRSDNH | PCL54 | |
| (SEQ ID NO: 238) | | |
| ISSGLLSSGGSGGSLSGRSGNH | PCL55 | |
| (SEQ ID NO: 239) | | |
| KGGPGGPAGIGPLAQRLRSSALF | PCL56 | FAPa_1, ADAM17_1, CTSL1_1 |
| KSSFPGR (SEQ ID NO: 240) | | |
| KSGPGGPAGIGALFFSSPPLAQK | PCL57 | FAPa_1, CTSL1_2, ADAM17_2 |
| LKSSGGR (SEQ ID NO: 241) | | |
| LSGRSDNHGGAVGLLAPP (SEQ | PCL58 | |
| ID NO: 242) | | |
| LSGRSDNHGGSGGSISSGLLSS | PCL59 | |
| (SEQ ID NO: 243) | | |
| LSGRSDNHGGSGGSQNQALRMA | PCL60 | |
| (SEQ ID NO: 244) | | |
| LSGRSDNHGGVHMPLGFLGP | PCL61 | |
| (SEQ ID NO: 245) | | |
| LSGRSGNHGGSGGSISSGLLSS | PCL62 | |
| (SEQ ID NO: 246) | | |
| LSGRSGNHGGSGGSQNQALRMA | PCL63 | |
| (SEQ ID NO: 247) | | |
| QNQALRMAGGSGGSLSGRSDNH | PCL64 | |
| (SEQ ID NO: 248) | | |
| QNQALRMAGGSGGSLSGRSGNH | PCL65 | |
| (SEQ ID NO: 249) | | |
| RGGALFKSSFPLAQKLKSSGPAG | PCL66 | CTSL1_1, ADAM17_2, MMP14_1 |
| LYAQGGK (SEQ ID NO: 250) | | |
| RGGGPAGLYAQPLAQKLKSSALF | PCL67 | MMP14_1, ADAM17_2, CTSL1_1 |
| KSSFPGG (SEQ ID NO: 251) | | |
| SGGFPRSGGSFNPRTFGSKRKRR | PCL68 | thrombin, factor Xa, hepsin |
| GSRGGGG (SEQ ID NO: 252) | | |
| SGPLAQKLKSSGPAGLYAQALFK | PCL69 | ADAM17_2, MMP14_1, CTSL1_1 |
| SSFPGSK (SEQ ID NO: 253) | | |
| TSTSGRSANPRGGGAVGLLAPP | PCL70 | |
| (SEQ ID NO: 254) | | |
| TSTSGRSANPRGGGVHMPLGFLG | PCL71 | |
| P (SEQ ID NO: 255) | | |
| VHMPLGFLGPGGLSGRSDNH | PCL72 | |
| (SEQ ID NO: 256) | | |
| VHMPLGFLGPGGTSTSGRSANP | PCL73 | |
| RG (SEQ ID NO: 257) | | |
| SGRSAGGGSGRSAGGGSGRSA | PCL74 | uPA |
| (SEQ ID NO: 258) | | |
| HPVGLLARGGGHPVGLLARGGGS | PCL75 | MPA (MMP-2 and uPA) |
| GRSAGGGSGRSA (SEQ ID | | |
| NO: 259) | | |
| GPLGVRGK (SEQ ID NO: 260) | PCL76 | MMP-2 |
| HPVGLLAR (SEQ ID NO: 261) | PCL77 | MMP-2 |
| GPQGIAGQ (SEQ ID NO: 46) | PCL78 | MMP-2, MMP-9, and to some |
| | | degree MT1-MMP |
| VPMSMRGG (SEQ ID NO: 262) | PCL79 | MMP-9 and MMP-2 |
| IPVSLRSG (SEQ ID NO: 61) | PCL80 | MMP-2, and to some degree |
| | | MMP-9 or MMP-7 |
| RPFSMIMG (SEQ ID NO: 263) | PCL81 | MMP-9 and MMP-7, to some |
| | | degree MMP-3 |
| VPLSLTMG (SEQ ID NO: 264) | PCL82 | MMP-7, to some degree MMP-9, |
| | | MMP-2, MPT-1-MMP |
| VPLSLYSG (SEQ ID NO: 149) | PCL83 | MMP-2, MMP-9, MMP-7 |
| IPESLRAG (SEQ ID NO: 265) | PCL84 | MMP-2, MMP-7, MMP-9, to some |
| | | degree MPT-1-MMP |
| GGGISSGLLSGRSDNHGGGS | PCL85 | |
| (SEQ ID NO: 338) | | |
| GGGHPVGLLARGGGS (SEQ ID | PCL86 | |
| NO: 339) | | |
| GGGSGGGSGGGGISSGLLSGRSD | PCL87 | |
| NHGGGSGGGSGGS (SEQ ID | | |
| NO: 340) | | |
| GGGGISSGLLSGRSDNHGGGISS | PCL88 | |
| GLLSGRSDNHGGS (SEQ ID NO: | | |
| 341) | | |
| GGGSGGSIPVSLRSGGGISSGLL | PCL89 | |
| SGRSDNHGGSGGS (SEQ ID NO: | | |
| 342) | | |
| GGGSGGSVPLSLYSGGGISSGLL | PCL90 | |
| SGRSDNHGGSGGS (SEQ ID NO: | | |
| 343) | | |
| GGGSHPVGLLARGGGHPVGLLAR | PCL91 | |
| GGGHPVGLLARGS (SEQ ID | | |
| NO: 344) | | |
| GGGSHPVGLLARGGGHPVGLLAR | PCL92 | |
| GGSGRSAGGSGRS (SEQ ID | | |
| NO: 345) | | |
| GISSGLLSGRSDNHG (SEQ ID | PCL93 | |
| NO: 354) | | |
| GGGSISSGLLSGRSDNHGGGS | PCL94 | |
| (SEQ ID NO: 355) | | |

In certain aspects, the protease-cleavable linker comprises an amino acid sequence having up to 5, up to 4, up to 3, up to 2 or up to 1 amino acid substitution(s) as compared to the sequence set forth in Table D. Thus, in some embodiments, the protease-cleavable linker comprises or consists of any amino acid sequence in Table D with 1-5 amino acid substitutions as compared to the sequence set forth in Table D.

### 6.6. Non-Cleavable Linkers

In certain aspects, the present disclosure provides IL2 proproteins in which two or more components of an IL2 proprotein are connected to one another by a peptide linker. By way of example and not limitation, linkers can be used to connect an Fc domain and an IL2Rα moiety or different domains within a targeting moiety (e.g., VH and VL domains in an scFv).

Preferably, all linkers in the IL2 proprotein other than the protease-cleavable linker whose cleavage results in activation of IL2 are non-cleavable linkers (NCLs).

A non-cleavable linker can range from 2 amino acids to 60 or more amino acids, and in certain aspects a non-cleavable peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a non-cleavable linker is at least 5 amino acids, at least 6 amino acids or at least 7 amino acids in length and optionally is up to 30 amino acids, up to 40 amino acids, up to 50 amino acids or up to 60 amino acids in length.

In some embodiments of the foregoing, the non-cleavable linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the non-cleavable linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the non-cleavable linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

Charged (e.g., charged hydrophilic linkers) and/or flexible non-cleavable linkers are particularly preferred.

Examples of flexible non-cleavable linkers that can be used in the IL2 proproteins of the disclosure include those disclosed by Chen et al., 2013, Adv Drug Deliv Rev. 65(10): 1357-1369 and Klein et al., 2014, Protein Engineering, Design & Selection 27(10): 325-330. Particularly useful flexible non-cleavable linkers are or comprise repeats of glycines and serines, e.g., a monomer or multimer of GnS (SEQ ID NO: 266) or SGn (SEQ ID NO: 267), where n is an integer from 1 to 10, e.g., 1, 2, 3, 4, 5, 6, 7, 8, 9 or 10. In one embodiment, the non-cleavable linker is or comprises a monomer or multimer of repeat of G₄S (SEQ ID NO: 1) e.g., (GGGGS)ₙ (SEQ ID NO: 153).

Polyglycine non-cleavable linkers can suitably be used in the IL2 proproteins of the disclosure. In some embodiments, a peptide non-cleavable linker comprises two consecutive glycines (2Gly), three consecutive glycines (3Gly), four consecutive glycines (4Gly) (SEQ ID NO: 268), five consecutive glycines (5Gly) (SEQ ID NO: 269), six consecutive glycines (6Gly) (SEQ ID NO: 270), seven consecutive glycines (7Gly) (SEQ ID NO: 271), eight consecutive glycines (8Gly) (SEQ ID NO: 272) or nine consecutive glycines (9Gly) (SEQ ID NO: 273).

Exemplary non-cleavable linker sequences are set forth in Table E below.

TABLE E
Non-Cleavable Linker Sequences

| Linker Sequence | Designation |
|---|---|
| GGGGSLALGPGGGGGSLALGPGGGG | NCL1 |
| GSLALGPGGS (SEQ ID NO: 274) | |
| GGGGSGGGGSGGGGSGGGGSGGGGS | NCL2 |
| (SEQ ID NO: 275) | |
| (GGGGS)ₙ (SEQ ID NO: 153) | NCL3 |
| (GGGS)ₙ (SEQ ID NO: 154) | NCL4 |
| (GGS)ₙ | NCL5 |
| (GS)ₙ | NCL6 |
| (GSGGS)ₙ (SEQ ID NO: 155) | NCL7 |
| ADAAP (SEQ ID NO: 276) | NCL8 |
| ADAAPTVSIFP (SEQ ID NO: 277) | NCL9 |
| ADAAPTVSIFPP (SEQ ID NO: 278) | NCL10 |
| AKTTAP (SEQ ID NO: 279) | NCL11 |
| AKTTAPSVYPLAP (SEQ ID NO: 280) | NCL12 |
| AKTTPKLEEGEFSEARV (SEQ ID NO: | NCL13 |
| 281) | |
| AKTTPKLGG (SEQ ID NO: 282) | NCL14 |
| AKTTPP (SEQ ID NO: 283) | NCL15 |
| AKTTPPSVTPLAP (SEQ ID NO: 284) | NCL16 |
| ASTKGP (SEQ ID NO: 285) | NCL17 |
| ASTKGPSVFPLAPASTKGPSVFPLAP | NCL18 |
| (SEQ ID NO: 286) | |
| EGKSSGSGSESKST (SEQ ID NO: 287) | NCL19 |
| GEGESGEGESGEGES (SEQ ID NO: | NCL20 |
| 288) | |
| GEGESGEGESGEGESGEGES (SEQ ID | NCL21 |
| NO: 289) | |
| GEGGSGEGGSGEGGS (SEQ ID NO: | NCL22 |
| 290) | |
| GENKVEYAPALMALS (SEQ ID NO: 291) | NCL23 |
| GGEGSGGEGSGGEGS (SEQ ID NO: | NCL24 |
| 292) | |
| GGGESGGEGSGEGGS (SEQ ID NO: | NCL25 |
| 293) | |
| GGGESGGGESGGGES (SEQ ID NO: | NCL26 |
| 294) | |
| GGGGSGGGGS (SEQ ID NO: 156) | NCL27 |
| GGGGSGGGGSGGGGS (SEQ ID NO: | NCL28 |
| 157) | |
| GGGGSGGGGSGGGGSGGGGS (SEQ | NCL29 |
| ID NO: 158) | |
| GGGKSGGGKSGGGKS (SEQ ID NO: | NCL30 |
| 159) | |
| GGGKSGGKGSGKGGS (SEQ ID NO: | NCL31 |
| 160) | |
| GGGS (SEQ ID NO: 161) | NCL32 |
| GGGSG (SEQ ID NO: 162) | NCL33 |
| GGKGSGGKGSGGKGS (SEQ ID NO: | NCL34 |
| 163) | |
| GGS | NCL35 |
| GGSG (SEQ ID NO: 295) | NCL36 |
| GGSGG (SEQ ID NO: 296) | NCL37 |
| GGSGG (SEQ ID NO: 296) | NCL38 |
| GGSGGGGSG (SEQ ID NO: 297) | NCL39 |
| GGSGGGGSGGGGS (SEQ ID NO: 164) | NCL40 |
| GHEAAAVMQVQYPAS (SEQ ID NO: | NCL41 |
| 298) | |
| GKGGSGKGGSGKGGS (SEQ ID NO: | NCL42 |
| 299) | |
| GKGKSGKGKSGKGKS (SEQ ID NO: | NCL43 |
| 300) | |
| GKGKSGKGKSGKGKSGKGKS (SEQ ID | NCL44 |
| NO: 301) | |
| GKPGSGKPGSGKPGS (SEQ ID NO: | NCL45 |
| 302) | |
| GKPGSGKPGSGKPGSGKPSGS (SEQ | NCL46 |
| ID NO: 303) | |
| GPAKELTPLKEAKVS (SEQ ID NO: 304) | NCL47 |
| GSAGSAAGSGEF (SEQ ID NO: 305) | NCL48 |
| GSGGG (SEQ ID NO: 167) | NCL49 |
| GSGSG (SEQ ID NO: 168) | NCL50 |
| GSS | NCL51 |
| GSSG (SEQ ID NO: 169) | NCL52 |
| GSSGGSGGSG (SEQ ID NO: 170) | NCL53 |
| GSSGGSGGSGG (SEQ ID NO: 171) | NCL54 |
| GSSGGSGGSGGS (SEQ ID NO: 172) | NCL55 |
| GSSGGSGGSGGSG (SEQ ID NO: 173) | NCL56 |
| GSSGGSGGSGGSGGGS (SEQ ID NO: | NCL57 |
| 174) | |
| GSSGGSGGSGS (SEQ ID NO: 175) | NCL58 |
| GSSGT (SEQ ID NO: 176) | NCL59 |
| GSSSG (SEQ ID NO: 177) | NCL60 |
| GSTSGSGKPGSGEGSTKG (SEQ ID | NCL61 |
| NO: 306) | |
| GTAAAGAGAAGGAAAGAAG (SEQ ID | NCL62 |
| NO: 307) | |
| GTSGSSGSGSGGSGSGGGG (SEQ ID | NCL63 |
| NO: 308) | |
| IRPRAIGGSKPRVA (SEQ ID NO: 309) | NCL64 |
| KESGSVSSEQLAQFRSLD (SEQ ID NO: | NCL65 |
| 310) | |
| KTTPKLEEGEFSEAR (SEQ ID NO: 311) | NCL66 |
| PRGASKSGSASQTGSAPGS (SEQ ID | NCL67 |
| NO: 312) | |
| QPKAAP (SEQ ID NO: 313) | NCL68 |
| QPKAAPSVTLFPP (SEQ ID NO: 314) | NCL69 |
| RADAAAA(G4S)₄ (SEQ ID NO: 315) | NCL70 |
| RADAAAAGGPGS (SEQ ID NO: 316) | NCL71 |
| RADAAP (SEQ ID NO: 317) | NCL72 |
| RADAAPTVS (SEQ ID NO: 318) | NCL73 |
| SAKTTP (SEQ ID NO: 319) | NCL74 |
| SAKTTPKLEEGEFSEARV (SEQ ID NO: | NCL75 |
| 320) | |
| SAKTTPKLGG (SEQ ID NO: 321) | NCL76 |
| STAGDTHLGGEDFD (SEQ ID NO: 322) | NCL77 |
| TVAAP (SEQ ID NO: 323) | NCL78 |
| TVAAPSVFIFPP (SEQ ID NO: 324) | NCL79 |
| TVAAPSVFIFPPTVAAPSVFIFPP (SEQ | NCL80 |
| ID NO: 325) | |

In certain aspects, the IL2 proprotein of the disclosure may comprise a polypeptide chain comprising, in an N- to C-terminal orientation, a targeting moiety (or targeting moiety chain), a hinge domain and a CH2 domain, and a CH3 domain. Thus, the hinge domain connects the targeting moiety with the CH2 domain and can be said to constitute a type of linker. Exemplary hinge domains are set forth in Section 6.9.3.

### 6.7. Targeting Moiety

The incorporation of targeting moieties in the IL2 proproteins of the disclosure permits the delivery of high concentrations of IL2 into the tumor microenvironment with a concomitant reduction of systemic exposure, resulting in fewer side effects than obtained with unmasked IL2 molecules.

It is anticipated that any type of target molecule present or capable of driving the IL2 proprotein at a particular locale or tissue may be targeted by the IL2 proproteins of the disclosure. In some embodiments, the IL2 proproteins are intended to treat cancer, e.g., by inducing a local immune response against tumor tissue. Accordingly, the targeting molecule can be any local tumor and associated target molecule. The target molecules recognized by the targeting moieties of the IL2 proproteins of the disclosure are generally found, for example, on the surfaces of activated T cells, on the surfaces of tumor cells, on the surfaces of virus-infected cells, on the surfaces of other diseased cells, free in blood serum, in the extracellular matrix (ECM), or immune cells present in the target site, e.g., tumor reactive lymphocytes.

In various aspects, a targeting moiety may bind to an extracellular matrix (“ECM”) antigen, a tumor reactive lymphocyte antigen, a cell surface molecule of tumor or viral lymphocytes, a T-cell antigen (“TCA”), a checkpoint inhibitor, or a tumor-associated antigen (“TAA”). The skilled artisan would recognize that the foregoing categories of target molecules are not mutually exclusive and thus a given target molecule may fall into more than one of the foregoing categories of target molecules. For example, some molecules may be considered both TAAs and ECM proteins, and other molecules may be considered both TCAs and checkpoint inhibitors.

Exemplary types of cancers that may be targeted include acute lymphoblastic leukemia, acute myelogenous leukemia, biliary cancer, B-cell leukemia, B-cell lymphoma, biliary cancer, bone cancer, brain cancer, breast cancer, triple-negative breast cancer, cervical cancer, Burkitt lymphoma, chronic lymphocytic leukemia, chronic myelogenous leukemia, colorectal cancer, endometrial cancer, esophageal cancer, gall bladder cancer, gastric cancer, gastrointestinal tract cancer, glioma, hairy cell leukemia, head and neck cancer, Hodgkin's lymphoma, liver cancer, lung cancer, medullary thyroid cancer, melanoma, multiple myeloma, ovarian cancer, non-Hodgkin's lymphoma, pancreatic cancer, prostate cancer, pulmonary tract cancer, renal cancer, sarcoma, skin cancer, testicular cancer, urothelial cancer, and other urinary bladder cancers. However, the skilled artisan will realize that TAAs and other target molecules associated with the tumor microenvironment are known for virtually any type of cancer.

Non-limiting examples of ECM antigens include syndecan, heparanase, integrins, osteopontin, link, cadherins, laminin, laminin type EGF, lectin, fibronectin, notch, nectin (e.g., nectin-4), tenascin, collagen (e.g., collagen type X) and matrixin.

Other target molecules are cell surface molecules of tumor or viral lymphocytes, for example T-cell co-stimulatory proteins such as CD27, CD28, 4-1BB (CD137), OX40, CD30, CD40, ICOS, lymphocyte function-associated antigen-1 (LFA-1), CD2, CD7, LIGHT, NKG2C, and B7-H3.

In particular embodiments, the target molecules are checkpoint inhibitors, for example CTLA-4, PD1, PDL1, PDL2, B7-H3, B7-H4, BTLA, HVEM, TIM3, GAL9, LAG3, VISTA, KIR, 2B4, CD160, CGEN-15049, CHK1, CHK2. In particular embodiments, the target molecule is PD1. In other embodiments, the target molecule is LAG3.

The antibodies and antigen-binding portions generally bind to specific antigenic determinants and are able to direct the IL2 proprotein to a target site, for example to a specific type of tumor cell or tumor stroma that bears the antigenic determinant. In particular embodiments, the targeting moiety recognizes a tumor-associated antigen (TAA). Preferably, the TAA is a human TAA. The antigen may or may not be present on normal cells. In certain embodiments, the TAA is preferentially expressed or upregulated on tumor cells as compared to normal cells. In other embodiments, the TAA is a lineage marker. Exemplary TAAs include Fibroblast Activation Protein (FAP), the A1 domain of Tenascin-C (TNC A1), the A2 domain of Tenascin-C (TNC A2), the Extra Domain B of Fibronectin (EDB), the Melanoma-associated Chondroitin Sulfate Proteoglycan (MCSP), MART-1/Melan-A, gp100, Dipeptidyl peptidase IV (DPPIV), adenosine deaminase-binding protein (ADAbp), cyclophilin b, colorectal associated antigen (CRC)-C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, c-erbB-2, Her2, EGFR, IGF-1R, CD2 (T-cell surface antigen), CD3 (heteromultimer associated with the TCR), CD22 (B-cell receptor), CD23 (low affinity IgE receptor), CD30 (cytokine receptor), CD33 (myeloid cell surface antigen), CD40 (tumor necrosis factor receptor), IL-6R-(IL6 receptor), CD20, MCSP, PDGFβR (β-platelet-derived growth factor receptor), ErbB2 epithelial cell adhesion molecule (EpCAM), EGFR variant Ill (EGFRvIII), CD19, disialoganglioside GD2, ductal-epithelial mucine, gp36, TAG-72, glioma-associated antigen, p-human chorionic gonadotropin, alphafetoprotein (AFP), lectin-reactive AFP, thyroglobulin, MN-CA IX, human telomerase reverse transcriptase, RU1, RU2 (AS), intestinal carboxyl esterase, mut hsp70-2, M-CSF, prostase, prostase specific antigen (PSA), PAP, LAGA-1a, p53, prostein, PSMA, surviving and telomerase, prostate-carcinoma tumor antigen-1 (PCTA-1), ELF2M, neutrophil elastase, ephrin B2, insulin growth factor (IGF1)-I, IGF-II, IGFI receptor, 5T4, ROR1, Nkp30, NKG2D, tumor stromal antigens, the extra domain A (EDA) and extra domain B (EDB) of fibronectin and the A1 domain of tenascin-C (TnC A1).

Suitable targeting moiety formats are described in Section 6.8. The targeting moiety is preferably an antigen binding moiety, for example an antibody or an antigen-binding portion of an antibody, e.g., an scFv, as described in Section 6.8.2 or a Fab, as described in Section 6.8.1.

In some embodiments, the targeting moieties target the exemplary target molecules set forth in Table F below, together with references to exemplary antibodies or antibody sequences upon which the targeting moiety can be based.

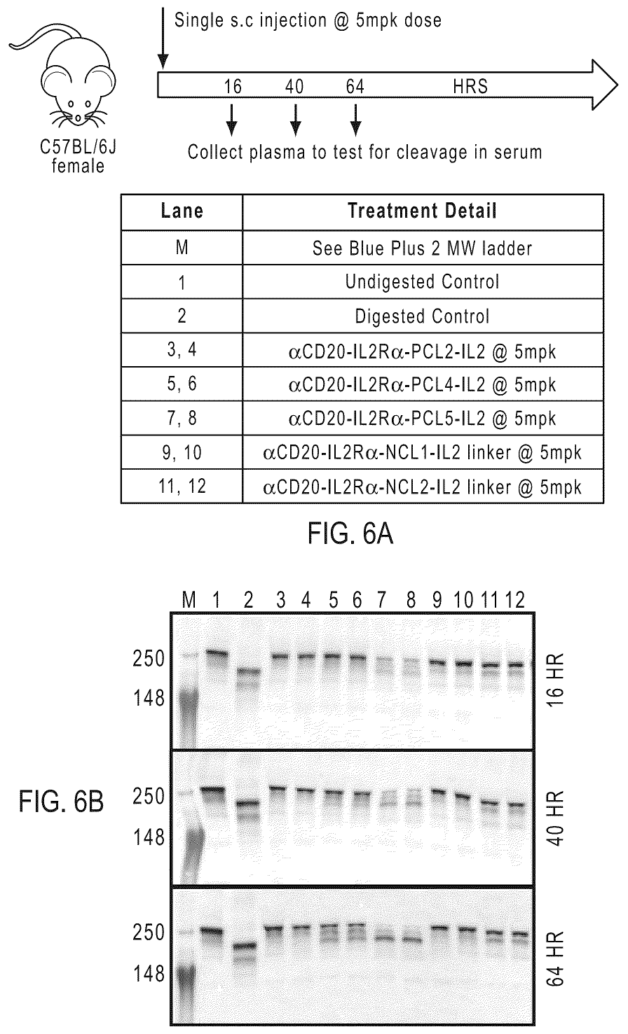

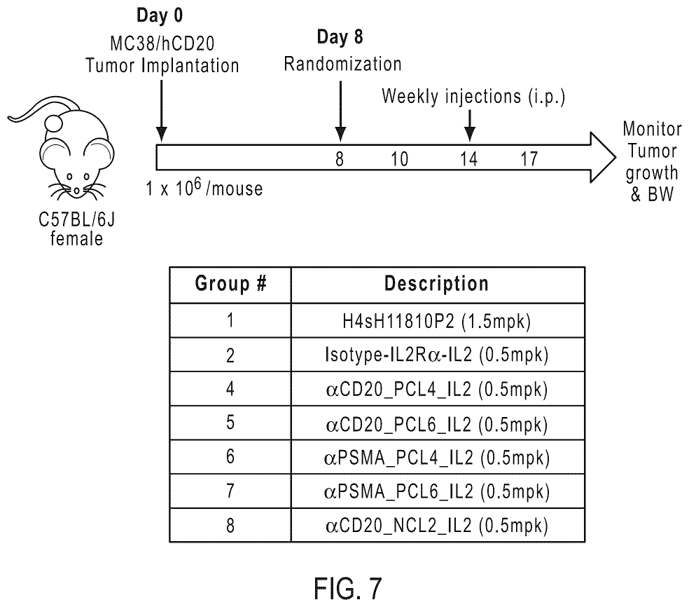

TABLE F
Exemplary Target Molecules

| Target | Antibody Name and/or Binding Sequences |
|---|---|
| 1-92-LFA-3 | Amevive ™ (alefacept) |
| 5T4 | GEN1044 |
| Activin Receptor Type II | Bimagrumab |
| | VH: SEQ ID NOs: 107, 109 of U.S. Pat. No. 8,388,968 B2 |
| | VL: SEQ ID NOs: 93, 95 of U.S. Pat. No. 8,388,968 B2 |
| B7-H3 | Obrindatamab (MGD009) |
| B7-H3 (CD276) | Enoblituzumab (MGA271) |
| B7-H3 (CD276) | MGC018 |
| B7-H3 (CD276) | MGA012 |
| B7-H3 (CD276) | 8H9 |
| B7-H3 (CD276) | VH: the VH sequence of the heavy chain of SEQ ID |
| | NO: 21, 26 or 31 of US 2021/0171641 A1. |
| | VL: the VL sequence of the light chain of SEQ ID NO: 20, |
| | 22 or 30 of US 2021/0171641 A1. |
| B7-H3 (CD276) | VH: the VH sequence of the heavy chain of SEQ ID |
| | NO: 21, 29 or 37 of US 2019/0002563 A1. |
| | VL: the VL sequence of the light chain of SEQ ID NO: 17, |
| | 25 or 33 of US 2019/0002563 A1. |
| B7-H3 (CD276) | VH: the VH sequence of the heavy chain of SEQ ID |
| | NO: 146, 147 or 148 of U.S. Pat. No. 10,640,563. |
| | VL: the VL sequence of the light chain of SEQ ID NO: 143, |
| | 144 or 145 of U.S. Pat. No. 10,640,563. |
| BAFF/B Lymphocyte | Benlysta ™ (velimumab) |
| Stimulator | |
| BAFF/B Lymphocyte | VH: amino acids 1-123 of SEQ ID NO: 327 of |
| Stimulator | U.S. Pat. No. 7,138,501 |
| | VL: amino acids 139-249 of SEQ ID NO: 327 of |
| | U.S. Pat. No. 7,138,501. |
| BAFF/B Lymphocyte | VH: amino acids 1-126 of SEQ ID NO: 1321 of |
| Stimulator | U.S. Pat. No. 7,605,236; |
| | VL: amino acids 143-251 of SEQ ID NO: 1049 of |
| | U.S. Pat. No. 7,605,236. |
| BAFF/B Lymphocyte | Belimumab |
| Stimulator | |
| BCMA | VH: the VH sequence of the heavy chain of SEQ ID NO. |
| | 126 of US 2021/0206865 A1 |
| | VL: the VL sequence of the light chain of SEQ ID NO. 129 |
| | or SEQ ID NO. 132 of US 2021/0206865 A1 |
| CA125 | Igobumab |
| CA125 | OvaRex ™ (oregobumab) |
| Cadherin | The antibodies described in US Pub. No. US |
| | 2006/0039915. |
| N-cadherin | An antibody that binds to the amino acid sequence of |
| | SEQ ID NO: 10, 17 or 18 of US Pub. No. US |
| | 2010/0278821. |
| CD11a | Raptiva ™ (efalizumab) |
| | Sequence in Werther et al., 1996, The Journal of |
| | Immunology 157(11): 4986-4995. |
| CD19 | Blincyto ™ (blinatumomab) |
| CD19 | SGN-CD19A |
| CD20 | Bexxar ™ (tositumomab) |
| | VH: the VH sequence of the heavy chain of SEQ ID |
| | NO: 124 of US Patent Pub. US 2017/0002060 A1 |
| | VL: the VL sequence of the light chain of SEQ ID NO: 125 |
| | of U.S. Patent Pub. US 2017/0002060 A1 |
| CD20 | Zevalin ™ (ibritumomab tiuxetan) |
| | VH: SEQ ID NO: 9 of U.S. Pat. No. 5,736,137 |
| | VL: SEQ ID NO: 6 of U.S. Pat. No. 5,736,137 |
| CD20 | Rituxan ™ (rituximab) |
| | VH: SEQ ID NO: 9 of U.S. Pat. No. 5,736,137 |
| | VL: SEQ ID NO: 6 of U.S. Pat. No. 5,736,137 |
| CD20 | Ocrevus ™ (ocrelizumab) |
| CD20 | Okaratuzumab |
| CD20 | Arzerra ™ (ofatumumab) |
| | VH: SEQ ID NO: 2 of U.S. Pat. No. 8,529,902 |
| | VL: SEQ ID NO: 4 of U.S. Pat. No. 8,529,902 |
| CD20 | Gazyva ™ (obinutuzumab) |
| CD20 | VH: SEQ ID NO: 4 of US 2021/0206870 A1 |
| | VL of SEQ ID NO: 6 of US 2021/0206870 A1 |
| CD20 | epcoritamab |
| CD22 | Belimumab |
| CD22 | Epratuzumab |
| CD22 | Besponsa ™ (inotuzumab ozogamicin) |
| CD22 | Lumoxiti ™ (moxetumumab pasudox) |
| CD22 | pinatuzumab vedotin |
| CD25 | Zenapax ™ (daclizumab) |
| | VH: SEQ ID NO: 9 of U.S. Pat. No. 7,060,269 |
| | VL: SEQ ID NO: 10 of U.S. Pat. No. 7,060,269 |
| CD30 | Adcetris ™ (brentuximab vedotin) |
| | VH: SEQ ID NO: 2 of U.S. Pat. No. 7,090,843 |
| | VL: SEQ ID NO: 10 of U.S. Pat. No. 7,090,843 |
| CD33 | Myelotarg ™ (gemtuzumab) |
| | Sequence in Man Sung, et al., 1993, Molecular |
| | immunology 30: 1361-1367 |
| CD33 | Lintuzumab |
| CD38 | Darzalex ™ (daratumumab) |
| CD40 | Lukatumumab |
| CD40 | Dacetuzumab |
| CD40L | Hu5c8 (ruplizumab) |
| CD44v6 | vibatuzumab mertansine |
| CD52 | Campath ™ (alemtuzumab) |
| | VH: SEQ ID NO: 1 of U.S. Patent Pub. US 2017/0002060 |
| | A1 |
| | VL: SEQ ID NO: 2 of U.S. Patent Pub. US 2017/0002060 |
| | A1 |
| CD70 | Blenrep ™ (borsetuzumab mafodotin) |
| CD123 | flotetuzumab |
| CD221 | Tepezza ™ (teprotumumab) |
| CEA | Hybri-Ceaker ® (altumomab pentetate) |
| CEA | Scintimun ™ (besilesomab) |
| CEA | CEA-CIDE ™ (labetuzumab)) |
| CEA | CEA-Scan ™ (arcitumomab) |
| CEA | hMN-15 |
| | CDR-H1, CDR-H2 and CDR-H3 sequences of SEQ ID |
| | NOs: 4-6 of U.S. Pat. No. 8,771,690 B2 |
| | CDR-L1, CDR-L2 and CDR-L3 sequences of SEQ ID |
| | NOs: 1-3 of U.S. Pat. No. 8,771,690 B2 |
| CEA | CEA binding portion of RO6958688/RG7802 from clinical |
| | trial NCT02324257 |
| CEA | Cibisatamab |
| CEA | CEA binding portion of MEDI-565/MT110/AMG211 from |
| | clinical trials NCT01284231 and NCT02291614 |
| | VH: SEQ ID NO: 49 or 51 of PCT Publication No. WO |
| | 2013/012414 A1 |
| | VL: SEQ ID NO: 48 of PCT Publication No. WO |
| | 2013/012414 A1. |
| CEA | Rabetuzumab |
| CEA | Atezolizumab |
| CEA | Cibisatamab |
| CEA | MEDI-565 (AMG211, MT111) |
| CEA | RO6958688 |
| CEA | VH: SEQ ID No. 9 described in WO2022/048883A1 |
| | VL: SEQ ID No. 10 described in WO2022/048883A1 |
| CLDN18.2 | AMG910 |
| Collagen alpha-4 chain | TRC093 (MT293) |
| Collagen | The collagen binding antibody fragment described in |
| | Liang, H. et al. A collagen-binding EGFR antibody |
| | fragment targeting tumors with a collagen-rich |
| | extracellular matrix. Sci. Rep. 5, 18205; doi: |
| | 10.1038/srep18205 (2016). |
| Collagen type I | Cetuximab (Erbitux) |
| Collagen type X | The amino acid sequences of SEQ ID NO: 1 or 2 of PCT |
| | Pub No. WO 2019/020797. |
| Collagen type X | The amino acid sequences of SEQ ID NO: 1 of PCT Pub |
| | No. WO 2014/180992. |
| Collagen type X | Antibody X34 as described in I. Girkontaite et al., |
| | “Immunolocalization of type X collagen in normal fetal and |
| | adult osteoarthritic cartilage with monoclonal |
| | antibodies,” Matrix Biol 15, 231-238 (1996). |
| Collagen type X | Antibodies X53 or 1H8 or ARC0659 or JF0961 collagen X |
| | polyclonal antibody sold under catalog number PA5- |
| | 115039 or PA5-116871 or PA5-97603 or PA5-49198 from |
| | ThermoFisher Scientific. |
| Collagen type X | Antibody sold under catalog number RDI-COLL10abr from |
| | RDI. |
| Complement C5 | Soliris ™ (eculizumab) |
| | VH: amino acids 1-122 of SEQ ID NO: 10 of |
| | U.S. Pat. No. 6,355,245 |
| | VL: amino acids 3-110 of SEQ ID NO: 9 of |
| | U.S. Pat. No. 6,355,245 |
| CTLA-4 | Yervoy ™ (ipilimumab) |
| | VH: SEQ ID NO: 17 of WO 2001/014424 A2 |
| | VL: SEQ ID NO: 7 of WO 2001/014424 A2 |
| CTLA-4 | (tremelimumab) |
| CTLA-4 | Orencia ™ (abatacept) |
| DLL3 | AMG757 |
| EGFR | Erbitux ™ (cetuximab) |
| | VH: SEQ ID NO: 11 of U.S. Pat. No. 6,217,866 |
| | VL: SEQ ID NO: 13 of U.S. Pat. No. 6,217,866 |
| EGFR | Vectibix ™ (panitumumab) |
| | VH: SEQ ID NO: 37 of U.S. Pat. No. 6,235,883 |
| | VL: SEQ ID NO: 38 of U.S. Pat. No. 6,235,883 |
| EGFR | Zalutumumab |
| | VH: SEQ ID NO: 64 of WO 2018/140831 A2 |
| | VL: SEQ ID NO: 69 of WO 2018/140831 A2 |
| EGFR | mapatumumab |
| EGFR | Matuzumab |
| EGFR | Nimotuzumab |
| | VH: SEQ ID NO: 51 of WO 2018/140831 A2 |
| | VL: SEQ ID NO: 56 of WO 2018/140831 A2 |
| EGFR | ICR62 |
| EGFR | mAb 528 |
| EGFR | CH806 |
| EGFRv3 | AMG596 |
| EGFRv3 | AMG404 |
| EGFR/CD64 | MDX-447 |
| EpCAM | Panorex ™ (edrecolomab) |
| | VH: SEQ ID NO: 129 of WO 2018/140831 A2 |
| | VL: SEQ ID NO: 134 of WO 2018/140831 A2 |
| EpCAM | Adecatumumab |
| | VH: SEQ ID NO: 142 of WO 2018/140831 A2 |
| | VL: SEQ ID NO: 147 of WO 2018/140831 A2 |
| EpCAM | tucotuzumab celmoleukin |
| EpCAM | citatuzumab bogatox |
| EpCAM | EP1629013 B1 |
| | VH: SEQ ID NOs: 80, 84, 88, 92 or 96 |
| | VL: SEQ ID NOs: 82, 86, 90, 94 or 98 |
| EpCAM | G8.8 |
| | HC: SEQ ID NO: 4 of U.S. Patent Pub. No. US |
| | 2020/0317806 A1 |
| | HL: SEQ ID NO: 3 of U.S. Patent Pub. No. US |
| | 2020/0317806 A1 |
| EpCAM | VH: SEQ ID NOs: 17-22 of WO 2021/211510 A2. |
| | VL: SEQ ID NO: 15-16 of WO 2021/211510 A2. |
| EpCAM | Removab ™(catumaxomab) |
| EpCAM | Vicineum ™(oportuzumab monatox) |
| EpCAM | M701 |
| F protein of RSV | Synagic ™ (palivizumab) |
| GD2 | 3F8 |
| Glycoprotein receptor IIb/IIIa | ReoPro ™ (abiciximab) |
| gpA33 | MGD007 |
| GPC3 | ERY974 |
| GUCY2C | PF-07062119 |
| Heparanase | An antibody selected from HP130, HP 239, HP 108.264, |
| | HP 115.140, HP 152.197, HP 110.662, HP 144.141, HP |
| | 108.371, HP 135.108, HP 151.316, HP 117.372, HP |
| | 37/33, HP3/17, HP 201 or HP 102 or an amino acid |
| | sequence of SEQ ID NO: 1-11 described in U.S. Pat. |
| | Pub. US 2004/0170631. |
| Her2 | Herceptin ™ (trastuzumab) |
| Her2 | Aldesleukin (proleukine) |
| Her2 | Sargramustim (leukine) |
| Her2 | M802 |
| Her2 | Runimotamab (BTRC4017A, R07227780) |
| Her2 | ISB1302 |
| Her2-neu | Perjeta ™ (pertuzumab) |
| | VH: SEQ ID NO: 16 of WO 2013/096812 A1. |
| | VL: SEQ ID NO: 15 of WO 2013/096812 A1. |
| Her2-neu | Rexomun ™ (ertumaxomab) |
| IgE | Xolair ™ (omalizumab) |
| IGFIR | (figitumumab) |
| IL1β | Ilaris ™ (canakinumab) |
| | VH: SEQ ID NO: 1 of U.S. Pat. No. 7,446,175. |
| | VL: SEQ ID NO: 2 of U.S. Pat. No. 7,446,175 |
| IL12/IL23 | Stelara ™ (ustekinumab) |
| IL1Ra | Antril ™, Kineret ™ (ankinra) |
| IL2R | Simulect ™ (basiliximab) |
| | VH: SEQ ID NO: 3 of U.S. Pat. No. 6,383,487 |
| | VL: SEQ ID NO: 6 of U.S. Pat. No. 6,383,487 |
| IL6 | Clazakizumab |
| IL6 receptor | Actemra ™ (tocilizumab) |
| | VH: SEQ ID NO: 31 of U.S. Pat. No. 7,479,543 |
| | VL: SEQ ID NO: 29 of U.S. Pat. No. 7,479,543 |
| IL12/IL23 p40 subunit | Stelara ™ (ustekinumab) |
| | VH: SEQ ID NO: 7 of U.S. Pat. No. 6,902,734 |
| | VL: SEQ ID NO: 8 of U.S. Pat. No. 6,902,734 |
| Integrin α4 | Tysabri ™ (natalizumab) |
| | VH: SEQ ID NOs: 11-13 of U.S. Pat. No. 5,840,299 |
| | VL: SEQ ID NOs: 7-8 of U.S. Pat. No. 5,840,299 |
| Integrin α4 β7 | Entyvio ™ (vedolizumab) |
| | HC: SEQ ID NO: 2 of U.S. Patent Pub. US 2012/0282249. |
| | LC: SEQ ID NO: 4 of U.S. Patent Pub. US 2012/0282249. |
| Integrin α5 β1 | VH: SEQ ID NO: 2 of European Patent No. 1 755 659. |
| | VL: SEQ ID NO: 4 of European Patent No. 1 755 659. |
| Integrin β1 | VH: SEQ ID NO: 2, 6, 8, 10, 12, 14, 29-43 or 91-100 of US |
| | Patent Pub. US 2022/0089744. |
| | VL:, SEQ ID NO: 4, 16, 18, 20, 22, 44-57 or 107-116 of US |
| | Patent Pub. US 2022/0089744. |
| LAG-3 | Relatlimab (BMS-98016) |
| LAG-3 | Sym022 |
| LAG-3 | HLX26 |
| LAG-3 | TSR-033 |
| LAG-3 | ABL501 |
| LAG-3 | INCAGN02385 |
| LAG-3 | Fianlimab (REGN3767) |
| LAG-3 | RO7247669 |
| LAG-3 | EMB-02 |
| LAG-3 | FS118 |
| LAG-3 | GSK2831781 |
| LAG-3 | IBI323 |
| LAG-3 | IBI110 |
| LAG-3 | LAG525 |
| LAG-3 | XmAb ®22841 |
| LAG-3 | LBL-007 |
| LAG-3 | VH: SEQ ID NO: 1, 8, 10 or 12 of |
| | U.S. Pat. No. 9,902,772. |
| | VL: SEQ ID NO: 2, 3, 4, 5, 6, 7, 9, 11, 13 or 14 of |
| | U.S. Pat. No. 9,902,772. |
| LAG-3 | VH: SEQ ID NO: 182 of U.S. Patent Pub. US |
| | 2021/0095026. |
| | VL: SEQ ID NO: 88 of U.S. Patent Pub. US 2021/0095026. |
| Laminin | Lam-89 from Sigma Aldrich |
| Mesothelin | Amatuximab |
| Mesothelin | HPN536 |
| MUC1 | civatuzumab tetraxetane |
| MUC1 | Pankomab ™ (gatipotuzumab) |
| MUC1 | femtumumab |
| MUC1 | Cantuzumab ravtansine |
| MUC16 (CA125) | Anti-MUC16 antibodies having VH and VL sequences |
| | having the amino acid sequences of any one of the |
| | following SEQ ID NO: pairs from US 2018/0118848A1: |
| | 18/26; 82/858; 98/170 |
| MUC17 | AMG199 |
| Nectin-4 | Enfortumab (ASP7465, ASG-22CE, ASG-22ME) |
| | VH: SEQ ID NO: 3 of PCT Pub. WO 2021/151984. |
| | VL: SEQ ID NO: 4 of PCT Pub. WO 2021/151984. |
| Nectin-4 | SBT290 |
| Nectin-4 | VH: SEQ ID NO: 1 of U.S. Pat. No. 11,274,160. |
| | VL: SEQ ID NO: 2 of U.S. Pat. No. 11,274,160. |
| NGF | (tanezumab) |
| Osteopontin | HC: SEQ ID NO: 22 of PCT Pub. WO 2021/030209. |
| | LC: SEQ ID NO: 24 of PCT Pub. WO 2021/030209. |
| PD1 | MDX-1106/BMS-936558 (nivolumab), a human lgG4 |
| | mAb with the structure described in WHO Drug |
| | Information, Vol. 27, No. 1, pages 68-69 (2013) and |
| | whose heavy and light chain sequences are disclosed in |
| | FIG. 7 of US Pub. No. US20190270812A1 |
| PD1 | MK-3475 (pembrolizumab), a humanized lgG4 mAb with |
| | the structure described in WHO Drug Information, Vol. 27, |
| | No. 2, pages 161-162 (2013) and whose heavy and light |
| | chain sequences are disclosed in FIG. 6 of US Pub. No. |
| | US20190270812A1 |
| PD1 | REGN2810 (disclosed as H4H7798N in US Pub No. |
| | 20150203579) |
| PD1 | Anti-PD1 antibodies disclosed in Tables 1-3 of PCT Pub. |
| | WO2015112800A1, including but not limited to anti-PD1 |
| | antibodies having VH/VL pairs having SEQ ID NOs: 2/10, |
| | 18/26, 34/42, 50/58, 66/74, 82/90, 98/106, 1 14/122, |
| | 130/138, 146/154, 162/170, 178/186, 194/202, 210/202, |
| | 218/202, 226/202, 234/202, 242/202, 250/202, 258/202, |
| | 266/202, 274/202, 282/202, 290/202, 298/186, 306/186 |
| | and 314/186 of PCT Pub. WO2015112800A1. |
| PD1 | MEDI-0680 (AMP-514) |
| PD1 | PDR001 |
| PD1 | BGB-108 |
| PD1 | h409A11, described in WO2008/156712 |
| PD1 | h409A16, described in WO2008/156712 |
| PD1 | h409A17, described in WO2008/156712 |
| PD1 | Anti-PD1 antibodies described in U.S. Pat. No. 7,488,802 |
| PD1 | Anti-PD1 antibodies described in U.S. Pat. No. 7,521,051 |
| PD1 | Anti-PD1 antibodies described in U.S. Pat. No. 8,008,449 |
| PD1 | Anti-PD1 antibodies described in U.S. Pat. No. 8,354,509 |
| PD1 | Anti-PD1 antibodies described in U.S. Pat. No. 8,168,757 |
| PD1 | Anti-PD1 antibodies described in PCT Pub. No. |
| | W02004/004771 |
| PD1 | Anti-PD1 antibodies described in PCT Pub. No. |
| | W02004/056875 |
| PD1 | Anti-PD1 antibodies described in PCT Pub. No. |
| | W02004/072286 |
| PD1 | Anti-PD1 antibodies described in US Pub. No. |
| | US2011/0271358 |
| PDL1 | Durvalumab (MEDI4736) |
| PDL1 | Atezolizumab (Tecentriq, MPDL3280A)) |
| PDL1 | MDX 1105 (BMS-936559) |
| PDL1 | Avelumab |
| PDL1 | ZKAB001 (Socazolimab) |
| PDL1 | TQB2450 |
| PDL1 | MEDI4736 |
| PDL1 | HLX20 |
| PDL1 | KN035 |
| PDL1 | LY3434172 |
| PDL1 | LY3300054 |
| PDL1 | LDP |
| PDL1 | EMB-09 |
| PDL1 | ABL501 |
| PDL1 | INBRX-105 |
| PDL1 | SHR-1210 |
| PDL1 | STI-3031 (IMC-001) |
| PDL1 | MPDL3280A (RG7446) |
| PDL1 | KN035 |
| PDL1 | BGB-A333 |
| PDL1 | HLX301 |
| PDL1 | Y101D |
| PDL1 | ES101 |
| PDL1 | IBI322 |
| PDL1 | Envafolimab |
| PDL1 | VH: SEQ ID NO: 46, 48, 50 or 52 of |
| | U.S. Pat. No. 11,168,144. |
| | VL: SEQ ID NO: 58, 137 or 12 of |
| | U.S. Pat. No. 11,168,144. |
| PDL1 | VH: SEQ ID NO: 23, 124, 126, 127, 128, 130, 140 or 145 |
| | of U.S. Pat. No. 11,208,486. |
| | VL: SEQ ID NO: 24 or 125 of U.S. Pat. No. 11,208,486. |
| Phosphatidylserine | (bavituximab) |
| PSCA | GEM3PSCA |
| PSMA | huJ591 |
| PSMA | Anti-PSMA antibodies having VH and VL sequences |
| | having the amino acid sequences of any one of the |
| | following SEQ ID NO: pairs from WO 2017/023761A1: |
| | 2/1642; 10/1642; 18/1642; 26/1642; 34/1642; 42/1642; |
| | 50/1642; 58/1642; 66/1642; 74/1642; 82/1642; 90/1642; |
| | 98/1642; 106/1642; 1 14/1642; 122/130; and 138/146. |
| PSMA | An antibody such as: PSMA 3.7, PSMA 3.8, PSMA 3.9, |
| | PSMA 3.11, PSMA 5.4, PSMA 7.1, PSMA 7.3, PSMA |
| | 10.3, PSMA 1.8.3, PSMA A3.1.3, PSMA A3.3.1, Abgenix |
| | 4.248.2, Abgenix 4.360.3, Abgenix 4.7.1, Abgenix 4.4.1, |
| | Abgenix 4.177.3, Abgenix 4.16.1, Abgenix 4.22.3, |
| | Abgenix 4.28.3, Abgenix 4.40.2, Abgenix 4.48.3, Abgenix |
| | 4.49.1, Abgenix 4.209.3, Abgemx 4.219.3, Abgenix |
| | 4.288.1, Abgenix 4.333.1, Abgemx 4.54.1, Abgenix |
| | 4.153.1, Abgenix 4.232.3, Abgenix 4.292.3, Abgenix |
| | 4.304.1, Abgenix 4.78.1 and Abgenix 4.152.1 described in |
| | WO2003034903A2 |
| | A hybridoma cell line such as: PSMA 3.7 (PTA-3257), |
| | PSMA 3.8, PSMA 3.9 (PTA- 3258), PSMA 3.11 (PTA- |
| | 3269), PSMA 5.4 (PTA-3268), PSMA 7.1 (PTA-3292), |
| | PSMA 7.3 (PTA-3293), PSMA 10.3 (PTA-3247) , PSMA |
| | 1.8.3 (PTA-3906), PSMA A3.1.3 (PTA- 3904), PSMA |
| | A3.3.1 (PTA-3905), Abgenix 4.248.2 (PTA-4427), Abgenix |
| | 4.360.3 (PTA- 4428), Abgenix 4.7.1 (PTA-4429), Abgenix |
| | 4.4.1 (PTA-4556), Abgenix 4.177.3 (PTA-4557), Abgenix |
| | 4.16.1 (PTA-4357), Abgenix 4.22.3 (PTA-4358), Abgenix |
| | 4.28.3 (PTA-4359), Abgenix 4.40.2 (PTA-4360), Abgenix |
| | 4.48.3 (PTA-4361), Abgenix 4.49.1 (PTA-4362), Abgenix |
| | 4.209.3 (PTA-4365), Abgenix 4.219.3 (PTA-4366), |
| | Abgenix 4.288.1 (PTA-4367), Abgenix 4.333.1 (PTA- |
| | 4368), Abgenix 4.54.1 (PTA-4363), Abgenix 4.153.1 |
| | (PTA-4388), Abgenix 4.232.3 (PTA-4389), Abgenix |
| | 4.292.3 (PTA-4390), Abgenix 4.304.1 (PTA-4391), |
| | Abgenix 4.78.1 (PTA-4652), and Abgemx 4.152.1(PTA- |
| | 4653) described in WO 2003/034903A2. |
| | VH of SEQ ID NOs: 2-7 described in WO 2003/034903A2 |
| | VL of SEQ ID NOs: 8-13 described in WO 2003/034903A2 |
| PMSA | VH: SEQ ID NOs: 225, 239, 253, 267, 281, 295, 309, |
| | 323, 337, 351, 365, 379, 393, 407, 421, 435, 449, 463, |
| | 477, 491, 505, 519, 533, 547, 561, 575, 589, 603 or 617 |
| | described in WO 2011/121110A1. |
| | VL SEQ ID NOs: 230, 244, 258, 272, 286, 300, 314, 328, |
| | 342, 356, 370, 384, 398, 412, 426, 440, 454, 468, 482, |
| | 496, 510, 524, 538, 552, 566, 580, 594, 608 or 622 |
| | described in WO 2011/121110A1. |
| | VH and VL SEQ ID Nos: 235, 249, 263, 277, 291, 305, |
| | 319, 333, 347, 361, 375, 389, 403, 417, 431, 445, 459, |
| | 473, 487, 501, 515, 529, 543, 557, 571, 585, 599, 613 or |
| | 627 described in WO 2011/121110A1. |
| PMSA | An anti-PMSA antibody having a VL amino acid sequence |
| | of any one of SEQ ID NOs: 229-312 of US 2022/0119525 |
| | A1 and a VH of SEQ ID NO: 217 of US 2022/0119525 A1. |
| PMSA | ES414 |
| PMSA | BAY2010112 (pasotuxizumab) |
| PMSA | CCW702 |
| PMSA | JNJ-63898081 |
| PMSA | CC-1 |
| PMSA | Acapatamab |
| PSMA | HPN424 |
| RAAG12 | RAV12 |
| RANKL | Prolia ™ (denosumab) |
| | VH: SEQ ID NO: 51 of U.S. Patent Pub. 2017/0002060 |
| | VL: SEQ ID NO: 52 of U.S. Patent Pub. 2017/0002060 |
| SLAMF7 | Empliciti ™ (elotuzumab) |
| SSTR2 | XmAb ®18087 |
| STEAP1 | VHCDR1 SEQ ID NOs: 14, 33, 182, 184 or 185 described |
| | in US20210179731A1. |
| | VHCDR2 SEQ ID NOs: 15, 21, 34, 182, 184 or 185 |
| | described in US20210179731A1. |
| | VHCDR3 SEQ ID NOs: 16 and 35 described in |
| | US20210179731A1. |
| | VH SEQ ID NOs: 182 or 184 described in |
| | US20210179731A1. |
| | VLCDR1 SEQ ID NOs: 11 or 30 described in |
| | US20210179731A1. |
| | VLCDR2 SEQ ID NOs: 12 or 31 described in |
| | US20210179731A1. |
| | VLCDR3 SEQ ID NOs: 13 or 32 described in |
| | US20210179731A1. |
| | VL SEQ ID NOs: 183 or 186 described in |
| | US20210179731A1. |
| STEAP1 | AMG509 |
| STEAP2 | Anti-STEAP 2 antibodies having CDR-H1, CDR-H2, CDR- |
| | H3, CDR-L1, CDR-L2 and CDR-L3 sequences selected |
| | from SEQ ID NOS: (1) 4-6-8-12-14-16; (2) 20-22-24-28- |
| | 30-32; (3) 36-38-40-44-46-48; (4) 52-54-56-60-62-64; (5) |
| | 68-70-72-60-62-64; (6) 76-78-80-60-62-64; (7) 84-86-88- |
| | 60-62-64; (8) 92-94-96-60-62-64; (9) 100-102-104-60-62- |
| | 64; (10) 108-110-112-116-118-120; (11) 124-126-128- |
| | 132-134-136; (12) 140-142-144-148-150-152; (13) 156- |
| | 158-160-164-166-168; (14) 172-174-176-180-182-184; |
| | (15) 188-190-192-196-198-200; (16) 204-206-208-212- |
| | 214-216; (17) 220-222-224-228-230-232; (18) 236-238- |
| | 240-244-246-248; (19) 252-254-256-260-262-264; (20) |
| | 268-270-272-276-278-280; (21) 284-286-288-292-294- |
| | 296; (22) 300-302-304-308-310-312; (23) 316-318-320- |
| | 324-326-328; (24) 332-334-336-340-342-344; (25) 348- |
| | 350-352-356-358-360; (26) 364-366-368-372-374-376; |
| | and (27) 380-382-384-388-390-392 of |
| | U.S. Pat. No. 10,772,972 B2. |
| | Anti-STEAP 2 antibodies having (a) a VH comprising the |
| | amino acid of any one of SEQ ID NOs: 2, 18, 34, 50, 66, |
| | 74, 82, 90, 98, 106, 122, 138, 154, 170, 186, 202, 218, |
| | 234, 250, 266, 282, 298, 314, 330, 346, 362, and 378 of |
| | U.S. Pat. No. 10,772,972 B2; and (b) a VL comprising |
| | the amino acid sequence of any one of SEQ ID NOs: 10; |
| | 26; 42; 58; 114; 130; 146; 162; 178; 194; 210; 226, 242; |
| | 258; 274; 290; 306; 322; 338; 354; 370; and 386 of |
| | U.S. Pat. No. 10,772,972 B2. |
| | Anti-STEAP 2 antibodies having a VH/VL pair comprising |
| | the amino acid sequences of any of the following pairs of |
| | SEQ ID NOs of U.S. Pat. No. 10,772,972 B2: 2/10; |
| | 18/26; 34/42; 50/58; 66/58; 74/58; 82/58; 90/58; 98/58; |
| | 106/114; 122/130; 138/146; 154/162; 170/178; 186/194; |
| | 202/210; 218/226; 234/242; 250/258; 266/274; 282/290; |
| | 298/306; 314/322; 330/338; 346/354; 362/370; and |
| | 378/386. |
| Syndecan-1 (CD 138) | The B-B4 antibody described in Wijdenes et al. (1996) Br. |
| | J. Haematol., 94: 318-323 |
| Syndecan-4 | The amino acid sequence of amino acids 93 and 121 of |
| | SEQ ID NO: 1 or the amino acid sequence of amino acids |
| | 92 and 122 of SEQ ID NO: 2 described in European Patent |
| | Pub. EP 2 603 236. |
| TGFβ | GC1008 |
| TNFR | Enbrel ™ (etanercept) |
| TNFα | Remicade ™ (infliximab) |
| | VH: SEQ ID NO: 2 of Int. Patent Publication |
| | WO201/3087911 A1 |
| | VH: SEQ ID NO: 3 of Int. Patent Publication WO2013/ |
| | A1087911 |
| TNFα | Humira ™ (adalimumab) |
| | VH: SEQ ID NO: 4 of U.S. Pat. No. 6,258,562 |
| | VL: SEQ ID NO: 3 of U.S. Pat. No. 6,258,562 |
| TNFα | Cimzia ™ (certolizumab pegol) |
| | VH: SEQ ID NO: 14 of U.S. Pat. No. 7,012,135 |
| | VL: SEQ ID NO: 9 of U.S. Pat. No. 7,012,135 |
| TNFα | Simponi ™ (golimumab) |
| | VH: SEQ ID NO: 7 of U.S. Pat. No. 7,250,165 |
| | VL: SEQ ID NO: 8 of U.S. Pat. No. 7,250,165 |
| VEGF | Avastin ™ (bevacizumab) |
| | VH: SEQ ID NO: 9 of U.S. Pat. No. 7,060,269 |
| | VL: SEQ ID NO: 10 of U.S. Pat. No. 7,060,269 |
| VEGF | Lucentis ™ (ranibizumab) |
| | VH: SEQ ID NO: 4 of U.S. Pat. No. 9,914,770 |
| | VL: SEQ ID NO: 2 of U.S. Pat. No. 9,914,770 |

In some aspects, the targeting moiety competes with an antibody set forth in Table F for binding to the target molecule. In further aspects, the targeting moiety comprises CDRs having CDR sequences of an antibody set forth in Table F. In some embodiments, the targeting moiety comprises all 6 CDR sequences of the antibody set forth in Table F. In other embodiments, the targeting moiety comprises at least the heavy chain CDR sequences (CDR-H1, CDR-H2, CDR-H3 and the light chain CDR sequences of a universal light chain. In further aspects, a targeting moiety comprises a VH comprising the amino acid sequence of the VH of an antibody set forth in Table F. In some embodiments, the targeting moiety further comprises a VL comprising the amino acid sequence of the VL of the antibody set forth in Table F. In other embodiments, the targeting moiety further comprises a universal light chain VL sequence.

In some embodiments, the targeting moiety is non-blocking or poorly-blocking of ligand-receptor binding. Examples of non-blocking or poorly-blocking anti-PD1 antibodies includes antibodies having VH/VL amino acid sequences of SEQ ID Nos: 2/10 of PCT Pub. No. WO2015/112800A1; SEQ ID Nos: 16/17 of U.S. Pat. No. 11,034,765 B2; SEQ ID Nos. 164/178, 165/179, 166/180, 167/181, 168/182, 169/183, 170/184, 171/185, 172/186, 173/187, 174/188, 175/189, 176/190 and 177/190 of U.S. Pat. No. 10,294,299 B2. Examples of non-blocking or poorly-blocking anti-LAG3 antibodies includes antibodies having VH/VL amino acid sequences of SEQ ID Nos 23/24, 3/4 and 11/12 of US Pub. US2022/0056126A1.

Additional target molecules that can be targeted by the IL2 proproteins are disclosed in Table I below and in, e.g., Hafeez et al., 2020, Molecules 25:4764, doi:10.3390/molecules25204764, particularly in Table 1. Table 1 of Hafeez et al. is incorporated by reference in its entirety here.

### 6.8. Targeting Moiety Formats

In certain aspects, the targeting moiety of an IL2 proprotein of the disclosure can be any type of antibody or fragment thereof that retains specific binding to an antigenic determinant. In one embodiment the targeting moiety is an immunoglobulin molecule or fragment thereof, particularly an IgG class immunoglobulin molecule, more particularly an IgG₁ or IgG₄ immunoglobulin molecule. Antibody fragments include, but are not limited to, VH (or V_{H}) fragments, VL (or V_{L}) fragments, Fab fragments, F(ab′)₂ fragments, scFv fragments, Fv fragments, minibodies, diabodies, triabodies, and tetrabodies.

### 6.8.1. Fab

Fab domains were traditionally produced by proteolytic cleavage of immunoglobulin molecules using enzymes such as papain. The Fab domains can comprise constant domain and variable region sequences from any suitable species, and thus can be murine, chimeric, human or humanized.

Fab domains typically comprise a CH1 domain attached to a VH domain which pairs with a CL domain attached to a VL domain. In a wild-type immunoglobulin, the VH domain is paired with the VL domain to constitute the Fv region, and the CH1 domain is paired with the CL domain to further stabilize the binding site. A disulfide bond between the two constant domains can further stabilize the Fab domain.

For the IL2 proproteins of the disclosure, particularly when the light chains of the targeting moieties are not common or universal light chains, it is advantageous to use Fab heterodimerization strategies to permit the correct association of Fab domains belonging to the same targeting moiety and minimize aberrant pairing of Fab domains belonging to different targeting moieties. For example, the Fab heterodimerization strategies shown in Table G below can be used:

TABLE G
Fab Heterodimerization Strategies

| STRATEGY | VH | CH1 | VL | CL | REFERENCE |
|---|---|---|---|---|---|
| CrossMabCH1- | WT | CL domain | WT | CH1 domain | Schaefer et al., 2011, |
| CL | | | | | Cancer Cell 2011; |
| | | | | | 20: 472-86; |
| | | | | | PMID: 22014573. |
| orthogonal Fab | 39K, 62E | H172A, F174G | 1R, 38D, (36F) | L135Y, S176W | Lewis et al., 2014, Nat |
| VHVRD1CH1CRD2 - | | | | | Biotechnol 32: 191-8 |
| VLVRD1CλCRD2 | | | | | |
| orthogonal Fab | 39Y | WT | 38R | WT | Lewis et al., 2014, Nat |
| VHVRD2CH1wt - | | | | | Biotechnol 32: 191-8 |
| VLVRD2Cλwt | | | | | |
| TCR CαCβ | 39K | TCR Cα | 38D | TCR Cβ | Wu et al., 2015, MAbs |
| | | | | | 7: 364-76 |
| CR3 | WT | T192E | WT | N137K, S114A | Golay at al., 2016, J |
| | | | | | Immunol 196: 3199-211. |
| MUT4 | WT | L143Q, S188V | WT | V133T, S176V | Golay at al., 2016, J |
| | | | | | Immunol 196: 3199-211. |
| DuetMab | WT | F126C | WT | S121C | Mazor et al., 2015, MAbs |
| | | | | | 7: 377-89; Mazor et al., |
| | | | | | 2015, MAbs 7: 461-669. |
| Domain | WT | CH3 + knob or | WT | CH3 + hole or | Wozniak-Knopp et al., |
| exchanged | | hole mutation | | knob mutation | 2018, |
| | | | | | PLOSONE13(4): e0195442 |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH:VL and CH1:CL interface such that the Fab components preferentially pair with each other rather than with components of other Fabs.

In one embodiment, the one or more amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179, the contents of which are hereby incorporated by reference.

In some embodiments, the Fab domain comprises a 192E substitution in the CH1 domain and 114A and 137K substitutions in the CL domain, which introduces a salt-bridge between the CH1 and CL domains (see, e.g., Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, e.g., Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014 Nature Biotechnology 32:191-198). In an embodiment, 39K, 62E modifications are introduced in the VH domain, H172A, F174G modifications are introduced in the CH1 domain, 1 R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121 C in the CL domain (see, e.g., Mazor et al., 2015, MAbs 7:377-89).

Fab domains can also be modified by replacing the CH1 domain and CL domain with alternative domains that promote correct assembly. For example, Wu et al., 2015, MAbs 7:364-76, describes substituting the CH1 domain with the constant domain of the T cell receptor and substituting the CL domain with the b domain of the T cell receptor, and pairing these domain replacements with an additional charge-charge interaction between the VL and VH domains by introducing a 38D modification in the VL domain and a 39K modification in the VH domain.

In lieu of, or in addition to, the use of Fab heterodimerization strategies to promote correct VH-VL pairings, the VL of common light chain (also referred to as a universal light chain) can be used for each unique ABD in the IL2 proproteins of the disclosure. In various embodiments, employing a common light chain as described herein reduces the number of inappropriate species in the IL2 proproteins as compared to employing original cognate VLs. In various embodiments, the VL domains of ABDs are identified from monospecific antibodies comprising a common light chain. In various embodiments, the VH regions of the ABDs in the IL2 proproteins comprise human heavy chain variable gene segments that are rearranged in vivo within mouse B cells that have been previously engineered to express a limited human light chain repertoire, or a single human light chain, cognate with human heavy chains and, in response to exposure with an antigen of interest, generate an antibody repertoire containing a plurality of human VHs that are cognate with one or one of two possible human VLs, wherein the antibody repertoire specific for the antigen of interest. Common light chains are those derived from a rearranged human VK1-39JK5 sequence or a rearranged human VK3-20JK1 sequence, and include somatically mutated (e.g., affinity matured) versions. See, for example, U.S. Pat. No. 10,412,940.

### 6.8.2. scFv

Single chain Fv or “scFv” antibody fragments comprise the VH and VL domains of an antibody in a single polypeptide chain, are capable of being expressed as a single chain polypeptide, and retain the specificity of the intact antibodies from which they are derived. Generally, the scFv polypeptide further comprises a polypeptide linker between the VH and VL domain that enables the scFv to form the desired structure for target binding. Examples of linkers suitable for connecting the VH and VL chains of an scFv are the non-cleavable linkers identified in Section v.

Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The scFv can comprise VH and VL sequences from any suitable species, such as murine, human or humanized VH and VL sequences.

To create an scFv-encoding nucleic acid, the VH and VL-encoding DNA fragments are operably linked to another fragment encoding a linker, e.g., encoding any of the linkers described in Section 6.6 (typically a repeat of a sequence containing the amino acids glycine and serine, such as the amino acid sequence (Gly4-Ser)3 (SEQ ID NO: 157), such that the VH and VL sequences can be expressed as a contiguous single-chain protein, with the VL and VH regions joined by the flexible linker (see, e.g., Bird et al., 1988, Science 242:423-426; Huston et al., 1988, Proc. Natl. Acad. Sci. USA 85:5879-5883; McCafferty et al., 1990, Nature 348:552-554).

### 6.9. Fc Regions

The IL2 proproteins of the disclosure typically include a pair of Fc domains that associate to form an Fc region. In native antibodies, Fc regions comprise hinge regions at their N-termini to form a constant domain. Throughout this disclosure, the reference to an Fc domain encompasses an Fc domain with a hinge domain at its N-terminus unless specified otherwise.

The Fc domains can be derived from any suitable species operably linked to an ABD or component thereof. In one embodiment the Fc domain is derived from a human Fc domain. In preferred embodiments, the targeting moiety or component thereof is fused to an IgG Fc molecule. A targeting moiety or component thereof may be fused to the N-terminus or the C-terminus of the IgG Fc domain or both.

The Fc domains can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgG1, IgG2, IgG3 or IgG4. In one embodiment the Fc domain is derived from IgG1. In one embodiment the Fc domain is derived from IgG4. Exemplary sequences of Fc domains from IgG1, IgG2, IgG3, and IgG4 are provided in Table Y, below.

TABLE Y
Fc Sequences

| | | SEQ |
|---|---|---|
| Fc | Sequence | ID NO |
| hIgG1 Fc | EPKSCDKTHTCPPCPAPELLGGPSVFLFPPKPKDTLMISRTPEVTCV | 346 |
| (amino | VVDVSHEDPEVKFNWYVDGVEVHNAKTKPREEQYNSTYRVVSVLTVL | |
| acids | HQDWLNGKEYKCKVSNKALPAPIEKTISKAKGQPREPQVYTLPPSRD | |
| 99-330 of | ELTKNQVSLTCLVKGFYPSDIAVEWESNGQPENNYKTTPPVLDSDGS | |
| UniprotKB | FFLYSKLTVDKSRWQQGNVFSCSVMHEALHNHYTQKSLSLSPGK | |
| P01857-1) | | |
| hIgG2 Fc | ERKCCVECPPCPAPPVAGPSVFLFPPKPKDTLMISRTPEVTCVVVDV | 347 |
| (amino | SHEDPEVQFNWYVDGVEVHNAKTKPREEQFNSTFRVVSVLTVVHQDW | |
| acids | LNGKEYKCKVSNKGLPAPIEKTISKTKGQPREPQVYTLPPSREEMTK | |
| 99-326 of | NQVSLTCLVKGFYPSDISVEWESNGQPENNYKTTPPMLDSDGSFFLY | |
| UniprotKB | SKLTVDKSRWQQGNVFSCSVMHEALHNHYTQKSLSLSPGK | |
| P01859-1) | | |
| hIgG3 Fc | ELKTPLGDTTHTCPRCPEPKSCDTPPPCPRCPEPKSCDTPPPCPRCP | 348 |
| (amino | EPKSCDTPPPCPRCPAPELLGGPSVFLFPPKPKDTLMISRTPEVTCV | |
| acids | VVDVSHEDPEVQFKWYVDGVEVHNAKTKPREEQYNSTFRVVSVLTVL | |
| 99-377 of | HQDWLNGKEYKCKVSNKALPAPIEKTISKTKGQPREPQVYTLPPSRE | |
| UniprotKB | EMTKNQVSLTCLVKGFYPSDIAVEWESSGQPENNYNTTPPMLDSDGS | |
| P01860-1) | FFLYSKLTVDKSRWQQGNIFSCSVMHEALHNRFTQKSLSLSPGK | |
| hIgG4 Fc | ESKYGPPCPSCPAPEFLGGPSVFLFPPKPKDTLMISRTPEVTCVVVD | 349 |
| (amino | VSQEDPEVQFNWYVDGVEVHNAKTKPREEQFNSTYRVVSVLTVLHQD | |
| acids | WLNGKEYKCKVSNKGLPSSIEKTISKAKGQPREPQVYTLPPSQEEMT | |
| 99-327 of | KNQVSLTCLVKGFYPSDIAVEWESNGQPENNYKTTPPVLDSDGSFFL | |
| UniprotKB | YSRLTVDKSRWQEGNVFSCSVMHEALHNHYTQKSLSLSLGK | |
| P01861-1) | | |

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO: 346. In cases where an Fc domain comprises at least 90% sequence identity and less than 100% sequence identity to SEQ ID NO: 346 (e.g., between 90% and 99% sequence identity to SEQ ID NO: 346), an Fc domain may also comprise one or more amino acid substitutions described herein, for example one or more substitutions that reduce effector function (e.g., as described in Section 6.9.1) and/or one or more substitutions that promote Fc heterodimerization (e.g., as described in Section 6.9.2).

In some aspects, an Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO: 347. In cases where an Fc domain comprises at least 90% sequence identity and less than 100% sequence identity to SEQ ID NO: 347 (e.g., between 90% and 99% sequence identity to SEQ ID NO: 347), an Fc domain may also comprise one or more amino acid substitutions described herein, for example one or more substitutions that reduce effector function (e.g., as described in Section 6.9.1) and/or one or more substitutions that promote Fc heterodimerization (e.g., as described in Section 6.9.2).

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO: 348. In cases where an Fc domain comprises at least 90% sequence identity and less than 100% sequence identity to SEQ ID NO: 348 (e.g., between 90% and 99% sequence identity to SEQ ID NO: 348), an Fc domain may also comprise one or more amino acid substitutions described herein, for example one or more substitutions that reduce effector function (e.g., as described in Section 6.9.1) and/or one or more substitutions that promote Fc heterodimerization (e.g., as described in Section 6.9.2).

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO: 349. In cases where an Fc domain comprises at least 90% sequence identity and less than 100% sequence identity to SEQ ID NO: 349 (e.g., between 90% and 99% sequence identity to SEQ ID NO: 349), an Fc domain may also comprise one or more amino acid substitutions described herein, for example one or more substitutions that reduce effector function (e.g., as described in Section 6.9.1) and/or one or more substitutions that promote Fc heterodimerization (e.g., as described in Section 6.9.2).

The two Fc domains within the Fc region can be the same or different from one another. In a native antibody the Fc domains are typically identical, but for the purpose of producing multispecific binding molecules, e.g., the IL2 proproteins of the disclosure and MBMs produced by their activation, the Fc domains might advantageously be different to allow for heterodimerization, as described in Section 6.9.2 below.

In native antibodies, the heavy chain Fc domain of IgA, IgD and IgG is composed of two heavy chain constant domains (CH2 and CH3) and that of IgE and IgM is composed of three heavy chain constant domains (CH2, CH3 and CH4). These dimerize to create an Fc region.

In IL2 proproteins of the present disclosure, the Fc region, and/or the Fc domains within it, can comprise heavy chain constant domains from one or more different classes of antibody, for example one, two or three different classes.

In one embodiment the Fc region comprises CH2 and CH3 domains derived from IgG1.

In one embodiment the Fc region comprises CH2 and CH3 domains derived from IgG2.

In one embodiment the Fc region comprises CH2 and CH3 domains derived from IgG3.

In one embodiment the Fc region comprises CH2 and CH3 domains derived from IgG4.

In one embodiment the Fc region comprises a CH4 domain from IgM. The IgM CH4 domain is typically located at the C-terminus of the CH3 domain.

In one embodiment the Fc region comprises CH2 and CH3 domains derived from IgG and a CH4 domain derived from IgM.

It will be appreciated that the heavy chain constant domains for use in producing an Fc region for the IL2 proproteins of the present disclosure may include variants of the naturally occurring constant domains described above. Such variants may comprise one or more amino acid variations compared to wild type constant domains. In one example the Fc region of the present disclosure comprises at least one constant domain that varies in sequence from the wild=type constant domain. It will be appreciated that the variant constant domains may be longer or shorter than the wild-type constant domain. Preferably the variant constant domains are at least 60% identical or similar to a wild-type constant domain. In another example the variant constant domains are at least 70% identical or similar. In another example the variant constant domains are at least 80% identical or similar. In another example the variant constant domains are at least 90% identical or similar. In another example the variant constant domains are at least 95% identical or similar.

IgM and IgA occur naturally in humans as covalent multimers of the common H2L2 antibody unit. IgM occurs as a pentamer when it has incorporated a J-chain, or as a hexamer when it lacks a J-chain. IgA occurs as monomer and dimer forms. The heavy chains of IgM and IgA possess an 18 amino acid extension to the C-terminal constant domain, known as a tailpiece. The tailpiece includes a cysteine residue that forms a disulfide bond between heavy chains in the polymer, and is believed to have an important role in polymerization. The tailpiece also contains a glycosylation site. In certain embodiments, the IL2 proproteins of the present disclosure do not comprise a tailpiece.

The Fc domains that are incorporated into the IL2 proproteins of the present disclosure may comprise one or more modifications that alter the functional properties of the proteins, for example, binding to Fc-receptors such as FcRn or leukocyte receptors, binding to complement, modified disulfide bond architecture, or altered glycosylation patterns. Exemplary Fc modifications that alter effector function are described in Section 6.9.1.

The Fc domains can also be altered to include modifications that improve manufacturability of asymmetric IL2 proproteins, for example by allowing heterodimerization, which is the preferential pairing of non-identical Fc domains over identical Fc domains. Heterodimerization permits the production of IL2 proproteins in which different polypeptide components are connected to one another by an Fc region containing Fc domains that differ in sequence. Examples of heterodimerization strategies are exemplified in Section 6.9.2.

It will be appreciated that any of the modifications mentioned above can be combined in any suitable manner to achieve the desired functional properties and/or combined with other modifications to alter the properties of the IL2 proproteins.

### 6.9.1. Fc Domains with Altered Effector Function

In some embodiments, the Fc domain comprises one or more amino acid substitutions that reduces binding to an Fc receptor and/or effector function.

In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In one embodiment, the Fc domain (e.g., an Fc domain of an IL2 proprotein half antibody) or the Fc region (e.g., one or both Fc domains of an IL2 proprotein that can associate to form an Fc region) comprises an amino acid substitution at a position selected from the group of E233, L234, L235, N297, P331 and P329 (numberings according to Kabat EU index). In a more specific embodiment, the Fc domain or the Fc region comprises an amino acid substitution at a position selected from the group of L234, L235 and P329 (numberings according to Kabat EU index). In some embodiments, the Fc domain or the Fc region comprises the amino acid substitutions L234A and L235A (numberings according to Kabat EU index). In one such embodiment, the Fc domain or region is an Igd Fc domain or region, particularly a human Igd Fc domain or region. In one embodiment, the Fc domain or the Fc region comprises an amino acid substitution at position P329. In a more specific embodiment, the amino acid substitution is P329A or P329G, particularly P329G (numberings according to Kabat EU index). In one embodiment, the Fc domain or the Fc region comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain or the Fc region comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”).

Typically, the same one or more amino acid substitution is present in each of the two Fc domains of an Fc region. Thus, in a particular embodiment, each Fc domain of the Fc region comprises the amino acid substitutions L234A, L235A and P329G (Kabat EU index numbering), i.e. in each of the first and the second Fc domains in the Fc region the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one embodiment, the Fc domain is an IgG1 Fc domain, particularly a human IgG1 Fc domain. In some embodiments, the IgG1 Fc domain is a variant IgG1 comprising D265A, N297A mutations (EU numbering) to reduce effector function.

In another embodiment, the Fc domain is an IgG4 Fc domain with reduced binding to Fc receptors. Exemplary IgG4 Fc domains with reduced binding to Fc receptors may comprise an amino acid sequence selected from Table H below: In some embodiments, the Fc domain includes only the bolded portion of the sequences shown below:

TABLE H

| Fc Domain | Sequence |
|---|---|
| SEQ ID NO: 332 | Asp Lys Arg Val Glu Ser Lys Tyr Gly Pro Cys Pro Pro Cys |
| (SEQ ID NO: 1 of | Pro Ala Pro Pro Val Ala Gly Pro Ser Val Phe Leu Phe Pro |
| WO2014/121087) | Pro Lys Pro Lys Asp Thr Leu Met Ile Ser Arg Thr Pro Glu |
| | Val Thr Cys Val Val Val Asp Val Ser Gln Glu Asp Pro Glu |
| | Val Gln Phe Asn Trp Tyr Val Asp Gly Val Glu Val His Asn |
| | Ala Lys Thr Lys Pro Arg Glu Glu Gln Phe Asn Ser Thr Tyr |
| | Arg Val Val Ser Val Leu Thr Val Leu His Gln Asp Trp Leu |
| | Asn Gly Lys Glu Tyr Lys Cys Lys Val Ser Asn Lys Gly Leu |
| | Pro Ser Ser Ile Glu Lys Thr Ile Ser Lys Ala Lys Gly Gln |
| | Pro Arg Glu Pro Gln Val Tyr Thr Leu Pro Pro Ser Gln Glu |
| | Glu Met Thr Lys Asn Gln Val Ser Leu Thr Cys Leu Val Lys |
| | Gly Phe Tyr Pro Ser Asp Ile Ala Val Glu Trp Glu Ser Asn |
| | Gly Gln Pro Glu Asn Asn Tyr Lys Thr Thr Pro Pro Val Leu |
| | Asp Ser Asp Gly Ser Phe Phe Leu Tyr Ser Arg Leu Thr Val |
| | Asp Lys Ser Arg Trp Gln Glu Gly Asn Val Phe Ser Cys Ser |
| | Val Met His Glu Ala Leu His Asn His Tyr Thr Gln Lys Ser |
| | Leu Ser Leu Ser Leu Gly Lys |
| SEQ ID NO: 333 | Asp Lys Lys Val Glu Pro Lys Ser Cys Asp Lys Thr His Thr |
| (SEQ ID NO: 2 of | Cys Pro Pro Cys Pro Ala Pro Pro Val Ala Gly Pro Ser Val |
| WO2014/121087) | Phe Leu Phe Pro Pro Lys Pro Lys Asp Thr Leu Met Ile Ser |
| | Arg Thr Pro Glu Val Thr Cys Val Val Val Asp Val Ser Gln |
| | Glu Asp Pro Glu Val Gln Phe Asn Trp Tyr Val Asp Gly Val |
| | Glu Val His Asn Ala Lys Thr Lys Pro Arg Glu Glu Gln Phe |
| | Asn Ser Thr Tyr Arg Val Val Ser Val Leu Thr Val Leu His |
| | Gln Asp Trp Leu Asn Gly Lys Glu Tyr Lys Cys Lys Val Ser |
| | Asn Lys Gly Leu Pro Ser Ser Ile Glu Lys Thr Ile Ser Lys |
| | Ala Lys Gly Gln Pro Arg Glu Pro Gln Val Tyr Thr Leu Pro |
| | Pro Ser Arg Asp Glu Leu Thr Lys Asn Gln Val Ser Leu Thr |
| | Cys Leu Val Lys Gly Phe Tyr Pro Ser Asp Ile Ala Val Glu |
| | Trp Glu Ser Asn Gly Gln Pro Glu Asn Asn Tyr Lys Thr Thr |
| | Pro Pro Val Leu Asp Ser Asp Gly Ser Phe Phe Leu Tyr Ser |
| | Lys Leu Thr Val Asp Lys Ser Arg Trp Gln Gln Gly Asn Val |
| | Phe Ser Cys Ser Val Met His Glu Ala Leu His Asn His Tyr |
| | Thr Gln Lys Ser Leu Ser Leu Ser Pro Gly Lys |
| SEQ ID NO: 334 | Ala Ser Thr Lys Gly Pro Ser Val Phe Pro Leu Ala Pro Ser |
| (SEQ ID NO: 30 | Ser Lys Ser Thr Ser Gly Gly Thr Ala Ala Leu Gly Cys Leu |
| of | Val Lys Asp Tyr Phe Pro Glu Pro Val Thr Val Ser Trp Asn |
| WO2014/121087) | Ser Gly Ala Leu Thr Ser Gly Val His Thr Phe Pro Ala Val |
| | Leu Gln Ser Ser Gly Leu Tyr Ser Leu Ser Ser Val Val Thr |
| | Val Pro Ser Ser Ser Leu Gly Thr Gln Thr Tyr Ile Cys Asn |
| | Val Asn His Lys Pro Ser Asn Thr Lys Val Asp Lys Lys Val |
| | Glu Pro Lys Ser Cys Asp Lys Thr His Thr Cys Pro Pro Cys |
| | Pro Ala Pro Pro Val Ala Gly Pro Ser Val Phe Leu Phe Pro |
| | Pro Lys Pro Lys Asp Thr Leu Met Ile Ser Arg Thr Pro Glu |
| | Val Thr Cys Val Val Val Asp Val Ser Gln Glu Asp Pro Glu |
| | Val Gln Phe Asn Trp Tyr Val Asp Gly Val Glu Val His Asn |
| | Ala Lys Thr Lys Pro Arg Glu Glu Gln Phe Asn Ser Thr Tyr |
| | Arg Val Val Ser Val Leu Thr Val Leu His Gln Asp Trp Leu |
| | Asn Gly Lys Glu Tyr Lys Cys Lys Val Ser Asn Lys Gly Leu |
| | Pro Ser Ser Ile Glu Lys Thr Ile Ser Lys Ala Lys Gly Gln |
| | Pro Arg Glu Pro Gln Val Tyr Thr Leu Pro Pro Ser Arg Asp |
| | Glu Leu Thr Lys Asn Gln Val Ser Leu Thr Cys Leu Val Lys |
| | Gly Phe Tyr Pro Ser Asp Ile Ala Val Glu Trp Glu Ser Asn |
| | Gly Gln Pro Glu Asn Asn Tyr Lys Thr Thr Pro Pro Val Leu |
| | Asp Ser Asp Gly Ser Phe Phe Leu Tyr Ser Lys Leu Thr Val |
| | Asp Lys Ser Arg Trp Gln Gln Gly Asn Val Phe Ser Cys Ser |
| | Val Met His Glu Ala Leu His Asn His Tyr Thr Gln Lys Ser |
| | Leu Ser Leu Ser Pro Gly Lys |
| SEQ ID NO: 335 | Ala Ser Thr Lys Gly Pro Ser Val Phe Pro Leu Ala Pro Cys |
| (SEQ ID NO: 31 | Ser Arg Ser Thr Ser Glu Ser Thr Ala Ala Leu Gly Cys Leu |
| of | Val Lys Asp Tyr Phe Pro Glu Pro Val Thr Val Ser Trp Asn |
| WO2014/121087) | Ser Gly Ala Leu Thr Ser Gly Val His Thr Phe Pro Ala Val |
| | Leu Gln Ser Ser Gly Leu Tyr Ser Leu Ser Ser Val Val Thr |
| | Val Pro Ser Ser Ser Leu Gly Thr Lys Thr Tyr Thr Cys Asn |
| | Val Asp His Lys Pro Ser Asn Thr Lys Val Asp Lys Arg Val |
| | Glu Ser Lys Tyr Gly Pro Pro Cys Pro Pro Cys Pro Ala Pro |
| | Pro Val Ala Gly Pro Ser Val Phe Leu Phe Pro Pro Lys Pro |
| | Lys Asp Thr Leu Met Ile Ser Arg Thr Pro Glu Val Thr Cys |
| | Val Val Val Asp Val Ser Gln Glu Asp Pro Glu Val Gln Phe |
| | Asn Trp Tyr Val Asp Gly Val Glu Val His Asn Ala Lys Thr |
| | Lys Pro Arg Glu Glu Gln Phe Asn Ser Thr Tyr Arg Val Val |
| | Ser Val Leu Thr Val Leu His Gln Asp Trp Leu Asn Gly Lys |
| | Glu Tyr Lys Cys Lys Val Ser Asn Lys Gly Leu Pro Ser Ser |
| | Ile Glu Lys Thr Ile Ser Lys Ala Lys Gly Gln Pro Arg Glu |
| | Pro Gln Val Tyr Thr Leu Pro Pro Ser Gln Glu Glu Met Thr |
| | Lys Asn Gln Val Ser Leu Thr Cys Leu Val Lys Gly Phe Tyr |
| | Pro Ser Asp Ile Ala Val Glu Trp Glu Ser Asn Gly Gln Pro |
| | Glu Asn Asn Tyr Lys Thr Thr Pro Pro Val Leu Asp Ser Asp |
| | Gly Ser Phe Phe Leu Tyr Ser Arg Leu Thr Val Asp Lys Ser |
| | Arg Trp Gln Glu Gly Asn Val Phe Ser Cys Ser Val Met His |
| | Glu Ala Leu His Asn His Tyr Thr Gln Lys Ser Leu Ser Leu |
| | Ser Leu Gly Lys |
| SEQ ID NO: 336 | Ala Ser Thr Lys Gly Pro Ser Val Phe Pro Leu Ala Pro Ser |
| (SEQ ID NO: 37 | Ser Lys Ser Thr Ser Gly Gly Thr Ala Ala Leu Gly Cys Leu |
| of | Val Lys Asp Tyr Phe Pro Glu Pro Val Thr Val Ser Trp Asn |
| WO2014/121087) | Ser Gly Ala Leu Thr Ser Gly Val His Thr Phe Pro Ala Val |
| | Leu Gln Ser Ser Gly Leu Tyr Ser Leu Ser Ser Val Val Thr |
| | Val Pro Ser Ser Ser Leu Gly Thr Gln Thr Tyr Ile Cys Asn |
| | Val Asn His Lys Pro Ser Asn Thr Lys Val Asp Lys Lys Val |
| | Glu Pro Lys Ser Cys Asp Lys Thr His Thr Cys Pro Pro Cys |
| | Pro Ala Pro Pro Val Ala Gly Pro Ser Val Phe Leu Phe Pro |
| | Pro Lys Pro Lys Asp Thr Leu Met Ile Ser Arg Thr Pro Glu |
| | Val Thr Cys Val Val Val Asp Val Ser Gln Glu Asp Pro Glu |
| | Val Gln Phe Asn Trp Tyr Val Asp Gly Val Glu Val His Asn |
| | Ala Lys Thr Lys Pro Arg Glu Glu Gln Phe Asn Ser Thr Tyr |
| | Arg Val Val Ser Val Leu Thr Val Leu His Gln Asp Trp Leu |
| | Asn Gly Lys Glu Tyr Lys Cys Lys Val Ser Asn Lys Gly Leu |
| | Pro Ser Ser Ile Glu Lys Thr Ile Ser Lys Ala Lys Gly Gln |
| | Pro Arg Glu Pro Gln Val Tyr Thr Leu Pro Pro Ser Arg Asp |
| | Glu Leu Thr Lys Asn Gln Val Ser Leu Thr Cys Leu Val Lys |
| | Gly Phe Tyr Pro Ser Asp Ile Ala Val Glu Trp Glu Ser Asn |
| | Gly Gln Pro Glu Asn Asn Tyr Lys Thr Thr Pro Pro Val Leu |
| | Asp Ser Asp Gly Ser Phe Phe Leu Tyr Ser Lys Leu Thr Val |
| | Asp Lys Ser Arg Trp Gln Gln Gly Asn Val Phe Ser Cys Ser |
| | Val Met His Glu Ala Leu His Asn Arg Phe Thr Gln Lys Ser |
| | Leu Ser Leu Ser Pro Gly Lys |
| SEQ ID NO: 337 | Ala Ser Thr Lys Gly Pro Ser Val Phe Pro Leu Ala Pro Cys |
| (SEQ ID NO: 38 | Ser Arg Ser Thr Ser Glu Ser Thr Ala Ala Leu Gly Cys Leu |
| of | Val Lys Asp Tyr Phe Pro Glu Pro Val Thr Val Ser Trp Asn |
| WO2014/121087) | Ser Gly Ala Leu Thr Ser Gly Val His Thr Phe Pro Ala Val |
| | Leu Gln Ser Ser Gly Leu Tyr Ser Leu Ser Ser Val Val Thr |
| | Val Pro Ser Ser Ser Leu Gly Thr Lys Thr Tyr Thr Cys Asn |
| | Val Asp His Lys Pro Ser Asn Thr Lys Val Asp Lys Arg Val |
| | Glu Ser Lys Tyr Gly Pro Pro Cys Pro Pro Cys Pro Ala Pro |
| | Pro Val Ala Gly Pro Ser Val Phe Leu Phe Pro Pro Lys Pro |
| | Lys Asp Thr Leu Met Ile Ser Arg Thr Pro Glu Val Thr Cys |
| | Val Val Val Asp Val Ser Gln Glu Asp Pro Glu Val Gln Phe |
| | Asn Trp Tyr Val Asp Gly Val Glu Val His Asn Ala Lys Thr |
| | Lys Pro Arg Glu Glu Gln Phe Asn Ser Thr Tyr Arg Val Val |
| | Ser Val Leu Thr Val Leu His Gln Asp Trp Leu Asn Gly Lys |
| | Glu Tyr Lys Cys Lys Val Ser Asn Lys Gly Leu Pro Ser Ser |
| | Ile Glu Lys Thr Ile Ser Lys Ala Lys Gly Gln Pro Arg Glu |
| | Pro Gln Val Tyr Thr Leu Pro Pro Ser Gln Glu Glu Met Thr |
| | Lys Asn Gln Val Ser Leu Thr Cys Leu Val Lys Gly Phe Tyr |
| | Pro Ser Asp Ile Ala Val Glu Trp Glu Ser Asn Gly Gln Pro |
| | Glu Asn Asn Tyr Lys Thr Thr Pro Pro Val Leu Asp Ser Asp |
| | Gly Ser Phe Phe Leu Tyr Ser Arg Leu Thr Val Asp Lys Ser |
| | Arg Trp Gln Glu Gly Asn Val Phe Ser Cys Ser Val Met His |
| | Glu Ala Leu His Asn Arg Phe Thr Gln Lys Ser Leu Ser Leu |
| | Ser Leu Gly Lys |

In a particular embodiment, the IgG4 with reduced effector function comprises the bolded portion of the amino acid sequence of SEQ ID NO:31 of WO2014/121087, sometimes referred to herein as IgG4s or hlgG4s.

For heterodimeric Fc regions, it is possible to incorporate a combination of the variant IgG4 Fc sequences set forth above, for example an Fc region comprising an Fc domain comprising the amino acid sequence of SEQ ID NO:30 of WO2014/121087 (or the bolded portion thereof) and an Fc domain comprising the amino acid sequence of SEQ ID NO:37 of WO2014/121087 (or the bolded portion thereof) or an Fc region comprising an Fc domain comprising the amino acid sequence of SEQ ID NO:31 of WO2014/121087 (or the bolded portion thereof) and an Fc domain comprising the amino acid sequence of SEQ ID NO:38 of WO2014/121087 (or the bolded portion thereof).

### 6.9.2. Fc Heterodimerization Variants

Certain IL2 proproteins entail dimerization between two Fc domains that, unlike a native immunoglobulin, are operably linked to non-identical N-terminal or C-terminal regions. Inadequate heterodimerization of two Fc domains to form an Fc region can be an obstacle for increasing the yield of desired heterodimeric molecules and represents challenges for purification. A variety of approaches available in the art can be used in for enhancing dimerization of Fc domains that might be present in the IL2 proproteins of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO 2009/089004A1.

In some embodiments, the present disclosure provides IL2 proproteins comprising Fc heterodimers, i.e., Fc regions comprising heterologous, non-identical Fc domains. Typically, each Fc domain in the Fc heterodimer comprises a CH3 domain of an antibody. The CH3 domains are derived from the constant region of an antibody of any isotype, class or subclass, and preferably of IgG (IgG1, IgG2, IgG3 and IgG4) class, as described in the preceding section.

Heterodimerization of the two different heavy chains at CH3 domains give rise to the desired IL2 proprotein, while homodimerization of identical heavy chains will reduce yield of the desired IL2 proprotein. Thus, in a preferred embodiment, the polypeptides that associate to form an IL2 proprotein of the disclosure will contain CH3 domains with modifications that favor heterodimeric association relative to unmodified Fc domains.

In a specific embodiment said modification promoting the formation of Fc heterodimers is a so-called “knob-into-hole” or “knob-in-hole” modification, comprising a “knob” modification in one of the Fc domains and a “hole” modification in the other Fc domain. The knob-into-hole technology is described e.g., in U.S. Pat. Nos. 5,731,168; 7,695,936; Ridgway et al., 1996, Prot Eng 9:617-621, and Carter, 2001, Immunol Meth 248:7-15. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g., by site-specific mutagenesis, or by peptide synthesis. An exemplary substitution is Y470T.

In a specific such embodiment, in the first Fc domain the threonine residue at position 366 is replaced with a tryptophan residue (T366W), and in the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further embodiment, in the first Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second Fc domain additionally the tyrosine residue at position 349 is replaced by a cysteine residue (Y349C) (numbering according to Kabat EU index). In a particular embodiment, the first Fc domain comprises the amino acid substitutions S354C and T366W, and the second Fc domain comprises the amino acid substitutions Y349C, T366S, L368A and Y407V (numbering according to Kabat EU index).

In some embodiments, electrostatic steering (e.g., as described in Gunasekaran et al., 2010, J Biol Chem 285(25): 19637-46) can be used to promote the association of the first and the second Fc domains of the Fc region.

As an alternative, or in addition, to the use of Fc domains that are modified to promote heterodimerization, an Fc domain can be modified to allow a purification strategy that enables selections of Fc heterodimers. In one such embodiment, one polypeptide comprises a modified Fc domain that abrogates its binding to Protein A, thus enabling a purification method that yields a heterodimeric protein. See, for example, U.S. Pat. No. 8,586,713. As such, the IL2 proproteins comprise a first CH3 domain and a second Ig CH3 domain, wherein the first and second Ig CH3 domains differ from one another by at least one amino acid, and wherein at least one amino acid difference reduces binding of the IL2 proprotein to Protein A as compared to a corresponding IL2 proprotein lacking the amino acid difference. In one embodiment, the first CH3 domain binds Protein A and the second CH3 domain contains a mutation/modification that reduces or abolishes Protein A binding such as an H95R modification (by IMGT exon numbering; H435R by EU numbering). The second CH3 may further comprise a Y96F modification (by IMGT; Y436F by EU). This class of modifications is referred to herein as “star” mutations.

In some embodiments, the Fc can contain one or more mutations (e.g., knob and hole mutations) to facilitate heterodimerization as well as star mutations to facilitate purification.

### 6.9.3. Hinge Domains

The IL2 proproteins of the disclosure can comprise an Fc domain comprising a hinge domain at its N-terminus. The hinge region can be a native or a modified hinge region. Hinge regions are typically found at the N-termini of Fc regions. The term “hinge domain”, unless the context dictates otherwise, refers to a naturally or non-naturally occurring hinge sequence that in the context of a single or monomeric polypeptide chain is a monomeric hinge domain and in the context of a dimeric polypeptide (e.g., a homodimeric or heterodimeric IL2 proprotein formed by the association of two Fc domains) can comprise two associated hinge sequences on separate polypeptide chains. Sometimes, the two associated hinge sequences are referred to as a “hinge region”.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions may comprise a complete hinge region derived from an antibody of a different class or subclass from that of the heavy chain Fc domain or Fc region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. Other modified hinge regions may be entirely synthetic and may be designed to possess desired properties such as length, cysteine composition and flexibility.

A number of modified hinge regions have already been described for example, in U.S. Pat. No. 5,677,425, WO 99/15549, WO 2005/003170, WO 2005/003169, WO 2005/003170, WO 98/25971 and WO 2005/003171 and these are incorporated herein by reference.

In one embodiment, an IL2 proprotein of the disclosure comprises an Fc region in which one or both Fc domains possesses an intact hinge domain at its N-terminus.

In various embodiments, positions 233-236 within a hinge region may be G, G, G and unoccupied; G, G, unoccupied, and unoccupied; G, unoccupied, unoccupied, and unoccupied; or all unoccupied, with positions numbered by EU numbering.

In some embodiments, the IL2 proproteins of the disclosure comprise a modified hinge region that reduces binding affinity for an Fcγ receptor relative to a wild-type hinge region of the same isotype (e.g., human IgG1 or human IgG4).

In one embodiment, the IL2 proproteins of the disclosure comprise an Fc region in which each Fc domain possesses an intact hinge domain at its N-terminus, where each Fc domain and hinge domain is derived from IgG4 and each hinge domain comprises the modified sequence CPPC. The core hinge region of human IgG4 contains the sequence CPSC compared to IgG1 that contains the sequence CPPC. The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide. (Angel et al., 1993, Mol Immunol 30(1):105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

### 6.9.3.1. Chimeric Hinge Sequences

The hinge domain can be a chimeric hinge domain.

For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region.

In particular embodiments, a chimeric hinge region comprises the amino acid sequence EPKSCDKTHTCPPCPAPPVA (SEQ ID NO: 326) (previously disclosed as SEQ ID NO:8 of WO2014/121087, which is incorporated by reference in its entirety herein) or ESKYGPPCPPCPAPPVA (SEQ ID NO: 327) (previously disclosed as SEQ ID NO:9 of WO2014/121087). Such chimeric hinge sequences can be suitably linked to an IgG4 CH2 region (for example by incorporation into an IgG4 Fc domain, for example a human or murine Fc domain, which can be further modified in the CH2 and/or CH3 domain to reduce effector function, for example as described in Section 6.9.1).

### 6.9.3.2. Hinge Sequences with Reduced Effector Function

In further embodiments, the hinge region can be modified to reduce effector function, for example as described in WO2016161010A2, which is incorporated by reference in its entirety herein. In various embodiments, the positions 233-236 of the modified hinge region are G, G, G and unoccupied; G, G, unoccupied, and unoccupied; G, unoccupied, unoccupied, and unoccupied; or all unoccupied, with positions numbered by EU numbering (as shown in FIG. 1 of WO2016161010A2). These segments can be represented as GGG-, GG--, G--- or ---- with “-” representing an unoccupied position.

Position 236 is unoccupied in canonical human IgG2 but is occupied by in other canonical human IgG isotypes. Positions 233-235 are occupied by residues other than G in all four human isotypes (as shown in FIG. 1 of WO2016161010A2).

The hinge modification within positions 233-236 can be combined with position 228 being occupied by P. Position 228 is naturally occupied by P in human IgG1 and IgG2 but is occupied by S in human IgG4 and R in human IgG3. An S228P mutation in an IgG4 antibody is advantageous in stabilizing an IgG4 antibody and reducing exchange of heavy chain light chain pairs between exogenous and endogenous antibodies. Preferably positions 226-229 are occupied by C, P, P and C respectively.

Exemplary hinge regions have residues 226-236, sometimes referred to as middle (or core) and lower hinge, occupied by the modified hinge sequences designated GGG-(233-236), GG--(233-236), G---(233-236) and no G(233-236). Optionally, the hinge domain amino acid sequence comprises CPPCPAPGGG-GPSVF (SEQ ID NO: 328) (previously disclosed as SEQ ID NO:1 of WO2016161010A2), CPPCPAPGG--GPSVF (SEQ ID NO: 329) (previously disclosed as SEQ ID NO:2 of WO2016161010A2), CPPCPAPG---GPSVF (SEQ ID NO: 330) (previously disclosed as SEQ ID NO:3 of WO2016161010A2), or CPPCPAP----GPSVF (SEQ ID NO: 331) (previously disclosed as SEQ ID NO:4 of WO2016161010A2).

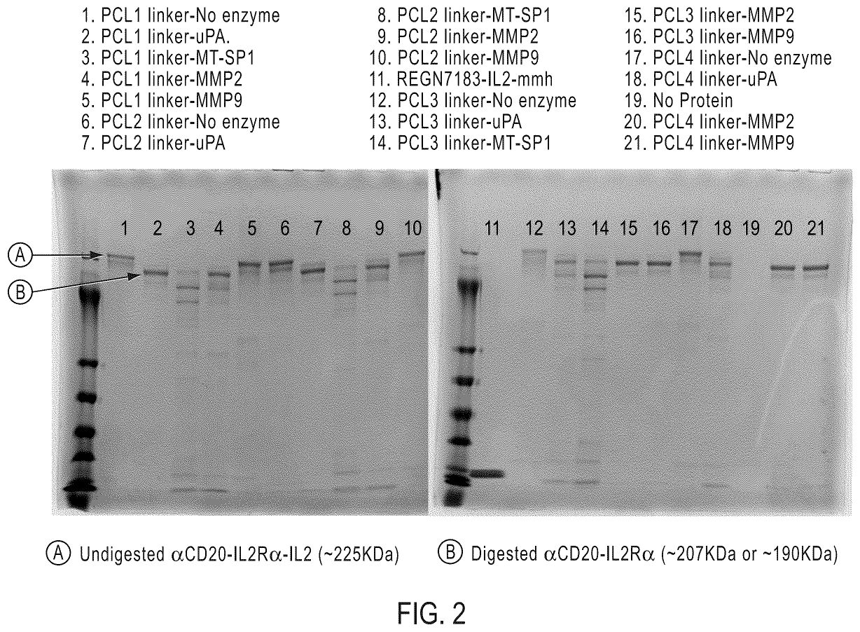

The modified hinge regions described above can be incorporated into a heavy chain constant region, which typically include CH2 and CH3 domains, and which may have an additional hinge segment (e.g., an upper hinge) flanking the designated region. Such additional constant region segments present are typically of the same isotype, preferably a human isotype, although can be hybrids of different isotypes. The isotype of such additional human constant regions segments is preferably human IgG4 but can also be human IgG1, IgG2, or IgG3 or hybrids thereof in which domains are of different isotypes. Exemplary sequences of human IgG1, IgG2 and IgG4 are shown in FIGS. 2-4 of WO2016161010A2.

In specific embodiments, the modified hinge sequences can be linked to an IgG4 CH2 region (for example by incorporation into an IgG4 Fc domain, for example a human or murine Fc domain, which can be further modified in the CH2 and/or CH3 domain to reduce effector function, for example as described in Section 6.9.1).

### 6.10. Nucleic Acids and Host Cells

In another aspect, the disclosure provides nucleic acids encoding the IL2 proproteins of the disclosure. In some embodiments, the IL2 proproteins are encoded by a single nucleic acid. In other embodiments, the IL2 proproteins can be encoded by a plurality (e.g., two, three, four or more) nucleic acids.

A single nucleic acid can encode an IL2 proprotein that comprises a single polypeptide chain, an IL2 proprotein that comprises two or more polypeptide chains, or a portion of an IL2 proprotein that comprises more than two polypeptide chains (for example, a single nucleic acid can encode two polypeptide chains of an IL2 proprotein comprising three, four or more polypeptide chains, or three polypeptide chains of an IL2 proprotein comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements, and separated by internal ribosome entry site (IRES) sequences allowing for translation into separate polypeptides.

In some embodiments, an IL2 proprotein comprising two or more polypeptide chains is encoded by two or more nucleic acids. The number of nucleic acids encoding an IL2 proprotein can be equal to or less than the number of polypeptide chains in the IL2 proprotein (for example, when more than one polypeptide chains are encoded by a single nucleic acid).

The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

### 6.10.1. Vectors

The disclosure provides vectors comprising nucleotide sequences encoding an IL2 proprotein or a component thereof described herein, for example one or two of the polypeptide chains of a half antibody of an IL2 proprotein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection or other conventional techniques. Methods and conditions for culturing the resulting transfected cells and for recovering the expressed polypeptides are known to those skilled in the art, and may be varied or optimized depending upon the specific expression vector and mammalian host cell employed, based upon the present description.

### 6.10.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### 6.11. Pharmaceutical Compositions

The IL2 proproteins of the disclosure may be in the form of compositions comprising the IL2 proprotein and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the IL2 proprotein and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically or locally. The most suitable route for administration in any given case will depend on the particular IL2 proprotein, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of an IL2 proprotein of the disclosure per dose. The quantity of IL2 proprotein included in a unit dose will depend on the disease being treated, as well as other factors as are well known in the art. Such unit dosages may be in the form of a lyophilized dry powder containing an amount of IL2 proprotein suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of IL2 proprotein suitable for a single administration.

The pharmaceutical compositions may also be supplied in bulk from containing quantities of IL2 proprotein suitable for multiple administrations.

Pharmaceutical compositions may be prepared for storage as lyophilized formulations or aqueous solutions by mixing an IL2 proprotein having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at a wide variety of concentrations, but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth, and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of IL2 proprotein.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188, etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

The IL2 proproteins of the disclosure can be formulated as pharmaceutical compositions comprising the IL2 proproteins, for example containing one or more pharmaceutically acceptable excipients or carriers. To prepare pharmaceutical or sterile compositions comprising the IL2 proproteins of the present disclosure, a IL2 proprotein preparation can be combined with one or more pharmaceutically acceptable excipient or carrier.

For example, formulations of IL2 proproteins can be prepared by mixing IL2 proproteins with physiologically acceptable carriers, excipients, or stabilizers in the form of, e.g., lyophilized powders, slurries, aqueous solutions, lotions, or suspensions (see, e.g., Hardman et al., 2001, Goodman and Gilman's The Pharmacological Basis of Therapeutics, McGraw-Hill, New York, N.Y.; Gennaro, 2000, Remington: The Science and Practice of Pharmacy, Lippincott, Williams, and Wlkins, New York, N.Y.; Avis, et al. (eds.), 1993, Pharmaceutical Dosage Forms: General Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y.).

An effective amount for a particular subject may vary depending on factors such as the condition being treated, the overall health of the subject, the method route and dose of administration and the severity of side effects (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

A composition of the present disclosure may also be administered via one or more routes of administration using one or more of a variety of methods known in the art. As will be appreciated by the skilled artisan, the route and/or mode of administration will vary depending upon the desired results. Selected routes of administration for IL2 proproteins include intravenous, intramuscular, intradermal, intraperitoneal, subcutaneous, spinal or other general routes of administration, for example by injection or infusion. General administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the disclosure can be administered via a non-general route, such as a topical, epidermal or mucosal route of administration, for example, intranasally, orally, vaginally, rectally, sublingually or topically. In one embodiment, the IL2 proproteins are administered by infusion. In another embodiment, the IL2 proprotein of the disclosure is administered subcutaneously.

### 6.12. Therapeutic Indications and Methods of Use

The present disclosure provides methods for using and applications for the IL2 proproteins of the disclosure.

In certain aspects, the disclosure provides a method of treating cancer, comprising administering to a subject in need thereof an IL2 proprotein or pharmaceutical composition as described herein. In some embodiments, an activated IL2 protein comprising the IL2 moiety is produced by cleavage of one or more protease-cleavable linkers in the IL2 proprotein by one or more proteases expressed by the cancer tissue. Accordingly, the IL2 proprotein is selectively activated in the cancer tissue.

In some embodiments, the disclosure provides a method of treating cancer with an IL2 protein that is selectively activated in cancer tissue, comprising administering to a subject in need thereof an IL2 proprotein or pharmaceutical composition as described herein, where the IL2 proprotein has one or more protease-cleavable linkers, each comprising one or more substrates for one or more proteases expressed by cancer tissue to which the IL2 protein is intended. Thus, an activated IL2 protein comprising the IL2 moiety is produced by cleavage of one or more protease-cleavable linkers in the IL2 proprotein by one or more proteases in the cancer tissue.

The present disclosure further provides a method of localized delivery of an IL2 protein, comprising administering to a subject an IL2 proprotein or pharmaceutical composition as described herein, where the IL2 proprotein has one or more protease-cleavable linkers, each comprising one or more substrates for one or more proteases expressed by a tissue to which the IL2 protein is to be locally delivered. As used herein, the term “locally delivered” does not require local administration but rather indicates that the active component of the IL2 proprotein refers to activation of the protein at a locale of interest by a protease active at the intended site, optionally in conjunction with targeting to the locale of interest with a targeting moiety that recognize a target molecule expressed by the tissue.

The present disclosure further provides a method of administering to the subject IL2 therapy with reduced systemic exposure and/or reduced systemic toxicity, comprising administering to a subject the IL2 therapy in the form of an IL2 proprotein or pharmaceutical composition as described herein, where the IL2 proprotein has one or more protease-cleavable linkers, each comprising one or more substrates for one or more proteases expressed by a tissue for which IL2 therapy is desirable and/or intended.

Accordingly, the foregoing methods permit IL2 therapy with reduced off-target side effects by virtue of preferential activation of an IL2 proprotein at a locale intended for IL2 treatment.

In some embodiments of the foregoing methods, the IL2 proprotein is also targeted and comprises one or more targeting moieties that recognize a target molecule expressed in the locale (e.g., by the tissue) intended for treatment.

Accordingly, the present disclosure provides a method of targeted delivery of an activated IL2 protein to a locale intended for treatment, e.g., cancer tissue, comprising administering to a subject an IL2 proprotein or pharmaceutical composition as described herein, wherein the IL2 comprises one or more targeting moieties that recognize a target molecule expressed in the locale or by the tissue intended for treatment (e.g., cancer tissue) and which has one or more protease-cleavable linkers, each comprising one or more substrates for one or more proteases expressed by a tissue for which IL2 therapy is desirable and/or intended.

The present disclosure further provides method of locally inducing an immune response in a target tissue, comprising administering to a subject IL2 proprotein or pharmaceutical composition as described herein which has one or more targeting moieties capable of binding a target molecule expressed in the target tissue and one or more protease-cleavable linkers, each comprising one or more substrates for one or more proteases expressed in the target tissue. An activated IL2 protein comprising the IL2 moiety can then be produced by cleavage of one or more protease-cleavable linkers in the IL2 proprotein by one or more proteases in the target tissue. The resulting activated IL2 protein can then induce the immune response against at least one cell type in the target tissue.

In some embodiments, the administration is not local to the tissue. For example, when the target tissue is cancer tissue, the administration can be systemic or subcutaneous.

The IL2 proproteins of the disclosure can be used in the treatment of any proliferative disorder (e.g., cancer) that expresses a target molecule (either on the tumor cells or in the tumor microenvironment, e.g., the extracellular matrix or the tumor lymphocytes). In particular embodiments, the cancer is acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, or Wilms tumor.

Table I below shows exemplary indications for which IL2 proproteins targeting particular target molecules can be used.

TABLE I
Examples of Target Molecule Indications

| Target | Exemplary Indication(s) |
|---|---|
| ADRB3 | Ewing sarcoma |
| ALK | NSCLC, ALCL, IMT, neuroblastoma |
| B7H3 | melanoma, osteosarcoma, leukemia, breast, prostate, ovarian, pancreatic, |
| | colorectal cancers |
| BCMA | multiple myeloma, leukemia (e.g., acute lymphoblastic leukemia (“ALL”), |
| | acute myeloid leukemia (“AML”), chronic lymphocytic leukemia (“CLL”), |
| | chronic myeloid leukemia (“CML”) and hairy cell leukemia (“HCL”)); |
| | lymphoma (e.g., Hodgkin's lymphoma, non-Hodgkin's lymphoma, including |
| | diffuse large B-cell lymphoma (“DLBCL”)) |
| Cadherin 17 | gastric, pancreatic, and colorectal adenocarcinomas |
| CAIX | clear-cell renal cell carcinoma, hypoxic solid tumors, head and neck |
| | squamous carcinoma |
| CD123 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CD171 | neuroblastoma, paraganglioma |
| CD179a | B cell malignancies |
| CD19 | leukemia (e.g., ALL, CLL, AML, CML, HCL); lymphoma (e.g., Hodgkin's |
| | lymphoma, non-Hodgkin's lymphoma, e.g., DLBCL); multiple myeloma. |
| CD20 | leukemia (e.g., ALL, CLL, AML, CML, HCL); lymphoma (e.g., Hodgkin's |
| | lymphoma, non-Hodgkin's lymphoma, e.g., DLBCL); multiple myeloma. |
| CD22 | leukemia (e.g., ALL, CLL, AML, CML, HCL); lymphoma (e.g., Hodgkin's |
| | lymphoma, non-Hodgkin's lymphoma, e.g., DLBCL); multiple myeloma; |
| | lung cancer |
| CD24 | ovarian, breast, prostate, bladder, renal, non-small cell carcinomas |
| CD30 | anaplastic large cell lymphoma, embryonal carcinoma, Hodgkin Lymphoma |
| CD32b | B cell malignancies, gastric, pancreatic, esophageal, glioblastoma, breast, |
| | colorectal |
| CD33 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CD38 | leukemia (e.g., ALL, CLL, AML, CML, HCL); lymphoma (e.g., Hodgkin's |
| | lymphoma, non-Hodgkin's lymphoma, e.g., DLBCL); multiple myeloma |
| CD44v6 | colon cancer, head and neck small cell carcinoma |
| CD97 | B cell malignancies, gastric, pancreatic, esophageal, glioblastoma, breast, |
| | colorectal |
| CEA | colorectal carcinoma, gastric carcinoma, pancreatic carcinoma, lung |
| (CEACAM5) | cancer, breast cancer, medullary thyroid carcinoma |
| CLDN6 | ovarian, breast, lung cancer |
| CLL-1 | leukemia (e.g., ALL, CLL, AML, CML, HCL); lymphoma (e.g., Hodgkin's |
| | lymphoma, non-Hodgkin's lymphoma, e.g., DLBCL); multiple myeloma. In |
| | a preferred embodiment, the indication is AML. |
| CS1 (SLAMF7) | multiple myeloma |
| EGFR | squamous cell carcinoma of lung, anal cancer, glioblastoma, epithelial |
| | tumors of head and neck, colon cancer |
| EGFRvIII | Glioblastoma |
| EPCAM | gastrointestestinal carcinoma, colorectal cancer |
| EphA2 | kaposi's sarcoma, glioblastoma, solid tumors, glioma |
| Ephrin B2 | thyroid cancer, breast cancer, malignant melanoma |
| ERBB2 | breast, ovarian, gastric cancers, lung adenocarcinoma, non-small cell lung |
| (Her2/neu) | cancer, uterine cancer, uterine serous endometrial carcinoma, salivary duct |
| | carcinoma |
| FAP | pancreatic cancer, colorectal cancer, metastasis, epithelial cancers, soft |
| | tissue sarcomas |
| FCRL5 | multiple myeloma |
| FLT3 | leukemia (e.g., ALL, CLL, AML, CML, HCL), lymphoma (e.g., Hodgkin's |
| | lymphoma, non-Hodgkin's lymphoma, e.g., DLBCL), multiple myeloma |
| Folate receptor | ovarian, breast, renal, lung, colorectal, brain cancers |
| alpha | |
| Folate receptor | ovarian cancer |
| beta | |
| Fucosyl GM1 | AML, myeloma |
| GD2 | malignant melanoma, neuroblastoma |
| GD3 | Melanoma |
| GloboH | ovarian, gastric, prostate, lung, breast, and pancreatic cancers |
| gp100 | Melanoma |
| GPNMB | breast cancer, head and neck cancers |
| GPR20 | GIST |
| GPR64 | Ewing sarcoma, prostate, kidney and lung sarcomas |
| GPRC5D | multiple myeloma |
| HAVCR1 | renal cancer |
| HER2 | HER-2 (+) adenocarcinoma of gastroesophageal junction, HER-2 positive |
| | gastric adenocarcinoma, HER2 positive carcinoma of breast |
| HER3 | colon and gastric cancers |
| HMWMAA | melanoma, glioblastoma, breast cancer |
| IGF-I receptor | breast, prostate, lung cancers |
| IL11Rα | papillary thyroid cancer, osteosarcoma, colorectal adenocarcinoma, |
| | lymphocytic leukemia |
| IL13Rα2 | renal cell carcinoma, prostate cancer, gliomas, head and neck cancer, |
| | astrocytoma |
| KIT | myeloid leukemia, kaposi's sarcoma, erythroleukemia, gastrointestinal |
| | stromal tumors |
| KLRG2 | breast cancers, lung cancers and ovarian cancers. |
| LewisY | squamous cell lung carcinoma, lung adenocarcinoma, ovarian carcinoma, |
| | and colorectal adenocarcinoma |
| LMP2 | prostate cancer, Hodgkin's lymphoma, nasopharyngeal carcinoma |
| LRP6 | breast cancer |
| LY6K | breast, lung, ovarian, and cervical cancer |
| LYPD8 | colorectal and gastric cancers |
| Mesothelin | mesothelioma, pancreatic cancer, ovarian cancer, stomach cancer, lung |
| | cancer, endometrial cancer |
| MUC1 | breast and ovarian cancers, lung, stomach, pancreatic, prostate cancers |
| NCAM | melanoma, Wilms' tumor, small cell lung cancer, neuroblastoma, myeloma, |
| | paraganglioma, pancreatic acinar cell carcinoma, myeloid leukemia |
| NY-BR-1 | breast cancer |
| o-acetyl GD2 | neuroblastoma, melanoma |
| OR51E2 | prostate cancer |
| PANX3 | Osteosarcoma |
| PLAC1 | hepatocellular carcinoma |
| Polysialic acid | small cell lung cancer |
| PDGFR-beta | myelomonocytic leukemia, chronic myeloid leukemia, acute myelogenous |
| | leukemia, acute lymphoblastic leukemia |
| PRSS21 | colon cancer, testicular cancer, ovarian cancer |
| PSCA | prostate cancer, gastric and bladder cancers |
| PSMA | prostate cancer |
| ROR1 | metastatic cancers, chronic lymphocytic leukemia, solid tumors in lung, |
| | breast, ovarian, colon, pancreatic, sarcoma |
| SLC34A2 | bladder cancer |
| SLC39A6 | breast cancer, esophageal cancer |
| SLITRK6 | breast cancer, urothelial cancer, lung cancer |
| SSEA-4 | breast cancer, cancer stem cells, epithelial ovarian carcinoma |
| STEAP1 | prostate cancer |
| STEAP2 | prostate cancer (including castrate-resistant prostate cancer), bladder |
| | cancer, cervical cancer, lung cancer, colon cancer, kidney cancer, breast |
| | cancer, pancreatic cancer, stomach cancer, uterine cancer, ovarian |
| | cancer, preferably prostate cancer |
| TACSTD2 | carcinomas, e.g., non-small-cell lung cancer |
| TAG72 | ovarian, breast, colon, lung, pancreatic cancers, gastric cancer |
| TEM1/CD248 | colorectal cancer |
| TEM7R | colorectal cancer |
| Tn | colorectal, breast cancers, cervical, lung, stomach cancers |
| TSHR | thyroid cancer, multiple myeloma |
| Tyrosinase | prostate cancer, melanoma |
| UPK2 | bladder cancer |
| VEGFR2 | ovarian and pancreatic cancers, renal cell carcinoma, colorectal cancer, |
| | medullary thyroid carcinoma |

Additional target molecules and corresponding indications are disclosed in, e.g., Hafeez et al., 2020, Molecules 25:4764, doi:10.3390/molecules25204764, particularly in Table 1. Table 1 is incorporated by reference in its entirety here.

### 7. SEQUENCES

Sequences of certain IL2 proproteins of the present disclosure are provided in Table S, below.

TABLE S
Example IL2 Proprotein Component Sequences

| | | SEQ ID |
|---|---|---|
| Component | Sequence | NO |
| hIgG1 Fc | EPKSCDKTHTCPPCPAPELLGGPSVFLFPPKPKDTLMISRTPEVTCVVVDVSHED | 346 |
| (amino acids | PEVKFNWYVDGVEVHNAKTKPREEQYNSTYRVVSVLTVLHQDWLNGKEYKCKVSN | |
| 99-330 of | KALPAPIEKTISKAKGQPREPQVYTLPPSRDELTKNQVSLTCLVKGFYPSDIAVE | |
| UniprotKB | WESNGQPENNYKTTPPVLDSDGSFFLYSKLTVDKSRWQQGNVFSCSVMHEALHNH | |
| P01857-1) | YTQKSLSLSPGK | |
| hIgG2 Fc | ERKCCVECPPCPAPPVAGPSVFLFPPKPKDTLMISRTPEVTCVVVDVSHEDPEVQ | 347 |
| (amino acids | FNWYVDGVEVHNAKTKPREEQFNSTFRVVSVLTVVHQDWLNGKEYKCKVSNKGLP | |
| 99-326 of | APIEKTISKTKGQPREPQVYTLPPSREEMTKNQVSLTCLVKGFYPSDISVEWESN | |
| UniprotKB | GQPENNYKTTPPMLDSDGSFFLYSKLTVDKSRWQQGNVFSCSVMHEALHNHYTQK | |
| P01859-1) | SLSLSPGK | |
| hIgG3 Fc | ELKTPLGDTTHTCPRCPEPKSCDTPPPCPRCPEPKSCDTPPPCPRCPEPKSCDTP | |
| (amino acids | PPCPRCPAPELLGGPSVFLFPPKPKDTLMISRTPEVTCVVVDVSHEDPEVOFKWY | |
| 99-377 of | KTISKTKGQPREPQVYTLPPSREEMTKNQVSLTCLVKGFYPSDIAVEWESSGQPE | 348 |
| UniprotKB | VDGVEVHNAKTKPREEQYNSTFRVVSVLTVLHQDWLNGKEYKCKVSNKALPAPIE | |
| P01860-1) | NNYNTTPPMLDSDGSFFLYSKLTVDKSRWQQGNIFSCSVMHEALHNRFTOKSLSL | |
| | SPGK | |
| hIgG4 Fc | ESKYGPPCPSCPAPEFLGGPSVFLFPPKPKDTLMISRTPEVTCVVVDVSQEDPEV | 349 |
| (amino acids | QFNWYVDGVEVHNAKTKPREEQFNSTYRVVSVLTVLHQDWLNGKEYKCKVSNKGL | |
| 99-327 of | PSSIEKTISKAKGQPREPQVYTLPPSQEEMTKNQVSLTCLVKGFYPSDIAVEWES | |
| UniprotKB | NGQPENNYKTTPPVLDSDGSFFLYSRLTVDKSRWQEGNVFSCSVMHEALHNHYTQ | |
| P01861-1) | KSLSLSLGK | |
| hIgG4s Fc | ESKYGPPCPPCPAPPVAGPSVFLFPPKPKDTLMISRTPEVTCVVVDVSQEDPEVQ | 350 |
| | FNWYVDGVEVHNAKTKPREEQFNSTYRVVSVLTVLHQDWLNGKEYKCKVSNKGLP | |
| | SSIEKTISKAKGQPREPQVYTLPPSQEEMTKNQVSLTCLVKGFYPSDIAVEWESN | |
| | GQPENNYKTTPPVLDSDGSFFLYSRLTVDKSRWQEGNVFSCSVMHEALHNHYTQK | |
| | SLSLSLGK | |
| hIgG1 PVA Fc | EPKSCDKTHTCPPCPAPPVAGPSVFLFPPKPKDTLMISRTPEVTCVVVDVSHEDP | 351 |
| | EVKFNWYVDGVEVHNAKTKPREEQYNSTYRVVSVLTVLHQDWLNGKEYKCKVSNK | |
| | ALPAPIEKTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMHEALHNHY | |
| | TQKSLSLSPGK | |
| hIL2 | APTSSSTKKTQLQLEHLLLDLQMILNGINNYKNPKLTRMLTFKFYMPKKATELKH | 2 |
| | LQCLEEELKPLEEVLNLAQSKNFHLRPRDLISNINVIVLELKGSETTFMCEYADE | |
| | TATIVEFLNRWITFCQSIISTLT | |
| hIL2Rα | ELCDDDPPEIPHATFKAMAYKEGTMLNCECKRGFRRIKSGSLYMLCTGNSSHSSW | 3 |
| extracellular | DNQCQCTSSATRNTTKQVTPQPEEQKERKTTEMOSPMQPVDQASLPGHCREPPPW | |
| domain | ENEATERIYHFVVGQMVYYQCVQGYRALHRGPAESVCKMTHGKTRWTQPQLICTG | |
| | EMETSQFPGEEKPQASPEGRPESETSCLVTTTDFQIQTEMAATMETSIFTTEYQV | |
| | AVAGCVFLLISVLLLSGLTWQRRQRKSRRTI | |
| amino acids | ELCDDDPPEIPHATFKAMAYKEGTMLNCECKRGFRRIKSGSLYMLCTGNSSHSSW | 4 |
| 22-186 of | DNQCQCTSSATRNTTKQVTPQPEEQKERKTTEMQSPMQPVDQASLPGHCREPPPW | |
| hIL2Rα | ENEATERIYHFVVGQMVYYQCVQGYRALHRGPAESVCKMTHGKTRWTQPQLICTG | |
| amino acids | ELCDDDPPEIPHATFKAMAYKEGTMLNCECKRGFRRIKSGSLYMLCTGNSSHSSW | 5 |
| 22-240 of | DNQCQCTSSATRNTTKQVTPQPEEQKERKTTEMOSPMQPVDQASLPGHCREPPPW | |
| hIL2Rα | ENEATERIYHFVVGQMVYYQCVQGYRALHRGPAESVCKMTHGKTRWTQPQLICTG | |
| | EMETSQFPGEEKPQASPEGRPESETSCLVTTTDFQIQTEMAATMETSIFTTEYQ | |
| hIgG1 CH1 | ASTKGPSVFPLAPSSKSTSGGTAALGCLVKDYFPEPVTVSWNSGALTSGVHTFPA | 352 |
| (amino acids | VLQSSGLYSLSSVVTVPSSSLGTQTYICNVNHKPSNTKVDKKV | |
| 1-98 of | | |
| UniprotKB | | |
| P01857-1) | | |
| hIgG2 CH1 | ASTKGPSVFPLAPCSRSTSESTAALGCLVKDYFPEPVTVSWNSGALTSGVHTFPA | 353 |
| (amino acids | VLQSSGLYSLSSVVTVPSSNFGTQTYTCNVDHKPSNTKVDKTV | |
| 1-98 of | | |
| UniprotKB | | |
| P01859-1) | | |
| hIgG3 CH1 | ASTKGPSVFPLAPCSRSTSGGTAALGCLVKDYFPEPVTVSWNSGALTSGVHTFPA | 354 |
| (amino acids | VLQSSGLYSLSSVVTVPSSSLGTQTYTCNVNHKPSNTKVDKRV | |
| 1-98 of | | |
| UniprotKB | | |
| P01860-1) | | |
| hIgG4 CH1 | ASTKGPSVFPLAPCSRSTSESTAALGCLVKDYFPEPVTVSWNSGALTSGVHTFPA | 355 |
| (amino acids | VLQSSGLYSLSSVVTVPSSSLGTKTYTCNVDHKPSNTKVDKRV | |
| 1-98 of | | |
| UniprotKB | | |
| P01861-1) | | |

### 8. NUMBERED EMBODIMENTS

While various specific embodiments have been illustrated and described, it will be appreciated that various changes can be made without departing from the spirit and scope of the disclosure(s). The present disclosure is exemplified by the numbered embodiments set forth below.

In the numbered embodiments that follow, the targeting moiety preferably binds to a mammalian target molecule, the IL2 and IL2Rα moieties are preferably derived from a mammalian IL2 and IL2Rα, the Fc domains are preferably derived from a mammalian antibody, and the subjects preferably mammals. More preferably, the mammal is human.
1. An IL2 proprotein comprising:
   (a) a first polypeptide chain comprising:
      (i) a first Fc domain;
      (ii) an optional first non-cleavable linker (NCL);
      (iii) a first IL2Rα moiety;
      (iv) a first protease-cleavable linker (PCL); and
      (v) a first IL2 moiety; and
   (b) a second polypeptide chain comprising:
      (i) a second Fc domain capable of associating with the first Fc domain to form an Fc region;
      (ii) an optional second non-cleavable linker (NCL);
      (iii) a second IL2Rα moiety;
      (iv) a second protease-cleavable linker (PCL); and
      (v) a second IL2 moiety.
2. The IL2 proprotein of embodiment 1, wherein the IL2 moiety comprises an amino acid sequence having at least about 90% sequence identity to mature human IL2.
3. The IL2 proprotein of embodiment 1, wherein the IL2 moiety comprises an amino acid sequence having about 95% sequence identity to mature human IL2.
4. The IL2 proprotein of any one of embodiments 1 to 3, wherein the IL2 moiety comprises an amino acid sequence having an N-terminal alanine deletion as compared to mature human IL2.
5. The IL2 proprotein of any one of embodiments 1 to 4, wherein the IL2 moiety comprises an amino acid sequence having an amino acid substitution at position N88 as compared to wild type IL2, optionally wherein the amino acid substitution is N88D.
6. The IL2 proprotein of any one of embodiments 1 to 5, wherein the IL2 moiety comprises an amino acid sequence having the amino acid substitution C125S, C125A or C125V as compared to wild type IL2.
7. The IL2 proprotein of any one of embodiments 1 to 6, wherein the IL2Rα moiety comprises or consists of an amino acid sequence having at least about 90% sequence identity to an IL2 binding portion of human IL2Rα.
8. The IL2 proprotein of any one of embodiments 1 to 6, wherein the IL2Rα moiety comprises or consists of an amino acid sequence having at least about 95% sequence identity to an IL2 binding portion of human IL2Rα.
9. The IL2 proprotein of any one of embodiments 1 to 6, wherein the IL2Rα moiety comprises or consists of an amino acid sequence having at least about 97% sequence identity to an IL2 binding portion of human IL2Rα.
10. The IL2 proprotein of any one of embodiments 1 to 6, wherein the IL2Rα moiety comprises or consists of an amino acid sequence having at least about 98% sequence identity to an IL2 binding portion of human IL2Rα.
11. The IL2 proprotein of any one of embodiments 1 to 6, wherein the IL2Rα moiety comprises or consists of an amino acid sequence having at least about 99% sequence identity to an IL2 binding portion of human IL2Rα.
12. The IL2 proprotein of any one of embodiments 1 to 6, wherein the IL2Rα moiety comprises or consists of an amino acid sequence having 100% sequence identity to an IL2 binding portion of human IL2Rα.
13. The IL2 proprotein of any one of embodiments 7 to 12, wherein the IL2Rα moiety comprises an amino acid sequence having at least 90% sequence identity to (a) amino acids 22-186 of IL2Rα, (b) amino acids 22-240 of IL2Rα, and/or (c) amino acids 22-272 of human IL2Rα.
14. The IL2 proprotein of any one of embodiments 7 to 12, wherein the IL2Rα moiety comprises an amino acid sequence having at least 95% sequence identity to (a) amino acids 22-186 of IL2Rα, (b) amino acids 22-240 of IL2Rα, and/or (c) amino acids 22-272 of human IL2Rα.
15. The IL2 proprotein of any one of embodiments 7 to 12, wherein the IL2Rα moiety comprises an amino acid sequence having at least 96% sequence identity to (a) amino acids 22-186 of IL2Rα, (b) amino acids 22-240 of IL2Rα, and/or (c) amino acids 22-272 of human IL2Rα.
16. The IL2 proprotein of any one of embodiments 7 to 12, wherein the IL2Rα moiety comprises an amino acid sequence having at least 97% sequence identity to (a) amino acids 22-186 of IL2Rα, (b) amino acids 22-240 of IL2Rα, and/or (c) amino acids 22-272 of human IL2Rα.
17. The IL2 proprotein of any one of embodiments 7 to 12, wherein the IL2Rα moiety comprises an amino acid sequence having at least 98% sequence identity to (a) amino acids 22-186 of IL2Rα, (b) amino acids 22-240 of IL2Rα, and/or (c) amino acids 22-272 of human IL2Rα.
18. The IL2 proprotein of any one of embodiments 7 to 12, wherein the IL2Rα moiety comprises an amino acid sequence having at least 99% sequence identity to (a) amino acids 22-186 of IL2Rα, (b) amino acids 22-240 of IL2Rα, and/or (c) amino acids 22-272 of human IL2Rα.
19. The IL2 proprotein of any one of embodiments 1 to 18, wherein the first PCL and/or second PCL comprises a substrate sequence cleavable by any protease set forth in Table A.
20. The IL2 proprotein of any one of embodiments 1 to 19, wherein the first PCL and/or second PCL comprises one or more substrate sequences selected from the substrate sequences set forth in Table B.
21. The IL2 proprotein of any one of embodiments 1 to 20 wherein the first PCL and/or second PCL comprises one or more spacer sequences selected from the substrate sequences set forth in Table C.
22. The IL2 proprotein of any one of embodiments 1 to 21, wherein the first PCL and/or second PCL comprises the amino acid sequence of any of the PCL sequences set forth in Table D or a variant thereof with up to 5 amino acid substitutions, e.g., a variant thereof with 1 amino acid substitution, 2 amino acid substitutions, 3 amino acid substitutions, 4 amino acid substitutions, or 5 amino acid substitutions.
23. The IL2 proprotein of any one of embodiments 1 to 22, wherein the first PCL and/or the second PCL comprises or consists of the amino acid sequence GGGISSGLLSGRSDNHGGGISSGLLSGRSDNHGGS (SEQ ID NO: 185).
24. The IL2 proprotein of any one of embodiments 1 to 22, wherein the first PCL and/or the second PCL comprises or consists of the amino acid sequence GGGISSGLLSGRSDNHGGGISSGLLSGRSDNHGGS GGGISSGLLSGRSDNHGGGISSGLLSGRSDNHGGS (SEQ ID NO: 186).
25. The IL2 proprotein of any one of embodiments 1 to 22, wherein the first PCL and/or the second PCL comprises or consists of the amino acid sequence GGSGGSIPVSLRSGGGISSGLLSGRSDNHGGSGGS (SEQ ID NO: 187).
26. The IL2 proprotein of any one of embodiments 1 to 22, wherein the first PCL and/or the second PCL comprises or consists of the amino acid sequence GGSGGSVPLSLYSGGGISSGLLSGRSDNHGGSGGS (SEQ ID NO: 188).
27. The IL2 proprotein of any one of embodiments 1 to 22, wherein the first PCL and/or the second PCL comprises or consists of the amino acid sequence GGSHPVGLLARGGGHPVGLLARGGGHPVGLLARGS (SEQ ID NO: 189).
28. The IL2 proprotein of any one of embodiments 1 to 22, wherein the first PCL and/or the second PCL comprises or consists of the amino acid sequence GGSHPVGLLARGGGHPVGLLARGGSGRSAGGSGRSA (SEQ ID NO: 190).
29. The IL2 proprotein of any one of embodiments 1 to 28, wherein the first NCL and/or second NCL comprises the amino acid sequence of any of the NCL sequences set forth in Table E or a variant thereof with up to 5 amino acid substitutions, e.g., a variant thereof with 1 amino acid substitution, 2 amino acid substitutions, 3 amino acid substitutions, 4 amino acid substitutions, or 5 amino acid substitutions.
30. The IL2 proprotein of any one of embodiments 1 to 28, wherein the first and second non-cleavable linkers range from 5 to 25 amino acids in length.
31. The IL2 proprotein of any one of embodiments 1 to 28, wherein the first and second non-cleavable linkers range from 7 to 20 amino acids in length.
32. The IL2 proprotein of any one of embodiments 1 to 28, wherein the first and second non-cleavable linkers are at least 5 amino acids in length.
33. The IL2 proprotein of any one of embodiments 1 to 28, wherein the first and second non-cleavable linkers are at least 7 amino acids in length.
34. The IL2 proprotein of any one of embodiments 1 to 34, wherein the first and second protease-cleavable linkers range from 20 amino acids to 80 amino acids in length.
35. The IL2 proprotein of any one of embodiments 1 to 34, wherein the first and second protease-cleavable linkers range from 20 amino acids to 60 amino acids in length.
36. The IL2 proprotein of any one of embodiments 1 to 35, wherein the first Fc domain and/or the second Fc domain comprises a hinge domain.
37. The IL2 proprotein of any one of embodiments 1 to 30, which further comprises one or more targeting moieties that bind to one or more target molecules.
38. The IL2 proprotein of embodiment 37, which comprises a first targeting moiety and/or a second targeting moiety.
39. The IL2 proprotein of embodiment 38, which comprises a first targeting moiety or component thereof (e.g., the VH of a Fab) N-terminal to the first Fc domain and/or a second targeting moiety or component thereof (e.g., the VH of a Fab) N-terminal to the second Fc domain.
40. The IL2 proprotein of embodiment 37 or embodiment 39, wherein the first targeting moiety and/or the second targeting moiety is a Fab.
41. The IL2 proprotein of embodiment 37 or embodiment 39, wherein the first targeting moiety and/or the second targeting moiety is an scFv.
42. The IL2 proprotein of any one of embodiments 37 to 41, wherein the first targeting moiety and/or second targeting moiety is capable of binding to an extracellular matrix (“ECM”) antigen, a tumor reactive lymphocyte antigen, a cell surface molecule of tumor or viral lymphocytes, or a checkpoint inhibitors or a tumor-associated antigen (“TAA”).
43. The IL2 proprotein of any one of embodiments 37 to 42, wherein the first targeting moiety and/or second targeting moiety is capable of binding to any target molecule identified in Section 6.7.
44. The IL2 proprotein of any one of embodiments 37 to 43, wherein the first targeting moiety and/or second targeting moiety (a) comprises the (i) CDR or (ii) VH and VL sequences of antibody set forth in Table F or (b) competes with the antibody set forth in Table F for binding to the target molecule.
45. The IL2 proprotein of any one of embodiments 37 to 43, wherein the first targeting moiety and/or second targeting moiety is capable of binding to an ECM antigen which is optionally selected from syndecan, heparanase, integrins, osteopontin, link, cadherins, laminin, laminin type EGF, lectin, fibronectin, notch, nectin (e.g., nectin-4), tenascin, collagen (e.g., collagen type X) and matrixin.
46. The IL2 proprotein of embodiment 45, wherein the first targeting moiety and/or second targeting moiety is capable of binding to a nectin, e.g., nectin 4.
47. The IL2 proprotein of embodiment 45, wherein the first targeting moiety and/or second targeting moiety is capable of binding to a collagen, e.g., collagen X.
48. The IL2 proprotein of any one of embodiments 37 to 43, wherein the first targeting moiety and/or second targeting moiety is capable of binding to a cell surface molecule of tumor or viral lymphocytes.
49. The IL2 proprotein of embodiment 48, wherein the antigen is a T-cell co-stimulatory protein.
50. The IL2 proprotein of embodiment 49, wherein the T-cell co-stimuatory protein is CD27, CD28, 4-1BB (CD137), OX40, CD30, CD40, ICOS, lymphocyte function-associated antigen-1 (LFA-1), CD2, CD7, LIGHT, NKG2C, or B7-H3.
51. The IL2 proprotein of embodiment 50, wherein the T-cell co-stimulatory protein is B7-H3.
52. The IL2 proprotein of any one of embodiments 37 to 43, wherein the first targeting moiety and/or second targeting moiety is capable of binding to a checkpoint inhibitor.
53. The IL2 proprotein of embodiment 52, wherein the checkpoint inhibitor is CTLA-4, PD1, PDL1, PDL2, B7-H3, B7-H4, BTLA, HVEM, TIM3, GAL9, LAG3, VISTA, KIR, 2B4, CD160, CGEN-15049, CHK1, or CHK2.
54. The IL2 proprotein of embodiment 53, wherein the checkpoint inhibitor is PDL1.
55. The IL2 proprotein of any one of embodiments 37 to 43, wherein the first targeting moiety and/or second targeting moiety is capable of binding to a tumor-associated antigen (“TAA”).
56. The IL2 proprotein of embodiment 55, wherein the first targeting moiety and/or second targeting moiety is capable of binding to AFP, ALK, a BAGE protein, BIRC5 (survivin), BIRC7, β-catenin, brc-abl, BRCA1, BORIS, CA9, carbonic anhydrase IX, caspase-8, CALR, CEACAM5 (also known as carcinoembryonic antigen or CEA), CCR5, CD19, CD20 (MS4A1), CD22, CD30, CD40, CDK4, CEA, CTLA4, cyclin-B1, CYP1B1, EGFR, EGFRvIII, ErbB2/Her2, ErbB3, ErbB4, ETV6-AML, EpCAM, EphA2, Fra-1, FOLR1, a GAGE protein (e.g., GAGE-1 or -2), GD2, GD3, GloboH, glypican-3, GM3, gp100, Her2, HLA/B-raf, HLA/k-ras, HLA/MAGE-A3, hTERT, LMP2, MAGE proteins (e.g., MAGE-1, -2, -3, -4, -6, and -12), MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, or uroplakin-3.
57. The IL2 proprotein of embodiment 56, wherein the TAA is EGFR.
58. The IL2 proprotein of embodiment 56, wherein the TAA is HER2.
59. The IL2 proprotein of embodiment 56, wherein the TAA is EPCAM.
60. The IL2 proprotein of embodiment 56, wherein the TAA is CEACAM5.
61. The IL2 proprotein of embodiment 56, wherein the TAA is CD20.
62. The IL2 proprotein of any one of embodiments 1 to 61, wherein the Fc region is homodimeric.
63. The IL2 proprotein of any one of embodiments 1 to 61, wherein the Fc region is heterodimeric.
64. The IL2 proprotein of any one of embodiments 1 to 63, wherein:
   (a) the first polypeptide chain comprises, in N- to C-terminal orientation:
      (i) the first Fc domain;
      (ii) optionally, the first non-cleavable linker,
      (iii) the first IL2Rα moiety;
      (iv) the first protease-cleavable linker; and
      (v) the first IL2 moiety; and
   (b) the second polypeptide chain comprises, in N- to C-terminal orientation:
      (i) the second Fc domain;
      (ii) optionally, the second non-cleavable linker,
      (iii) the second IL2Rα moiety;
      (iv) the second protease-cleavable linker; and
      (v) the second IL2 moiety; and
65. An IL2 proprotein, which is optionally an IL2 proprotein of any one of embodiments 1 to 64, wherein the IL2 proprotein comprises:
   (a) a first Fc domain and a second Fc domain capable of associating to form an Fc region;
   (b) two IL2Rα moieties C-terminal to the Fc domains optionally preceded by linkers, optionally non-cleavable linkers;
   (c) two protease-cleavable linkers C-terminal to the IL2Rα moieties; and
   (d) two IL2 moieties C-terminal to the protease-cleavable linkers
66. An IL2 proprotein according to any one of embodiments 1 to 64, wherein:
   (a) the first polypeptide comprises: the first Fc domain, optionally followed by the first non-cleavable linker, followed by the first IL2Rα moiety, followed by the first protease-cleavable linker, followed by the first IL2 moiety; and
   (b) the second polypeptide comprises: the second Fc domain, optionally followed by the second non-cleavable linker, followed by the second IL2Rα moiety, followed by the second protease-cleavable linker, followed by the second IL2 moiety.
67. An IL2 proprotein according to any one of embodiments 1 to 64, which has the configuration depicted in FIG. 1A.
68. An IL2 proprotein, which is optionally an IL2 proprotein of any one of embodiments 1 to 64, wherein the IL2 proprotein comprises:
   (a) a first polypeptide chain comprising, in N- to C-terminal order:
      (i) a first amino acid sequence having at least about 95% sequence identity to the amino acid sequence of any one of SEQ ID NOs: 346, 347, 348, 349, 350, and 351;
      (ii) a non-cleavable linker/non-cleavable means for connecting the first amino acid sequence to a third amino sequence, optionally wherein the linker/means comprises or consists of a second amino acid sequence comprising an amino acid sequence set forth in Table E;
      (iii) the third amino acid sequence, having at least about 95% sequence identity to the amino acid sequence of any one of SEQ ID NOs: 3, 4, and 5;
      (iv) a fourth amino acid sequence comprising one or more amino acid sequences set forth in Table B or Table D; and
      (v) a fifth amino acid sequence having at least about 95% sequence identity to the amino acid sequence of SEQ ID NO: 2; and
   (b) a second polypeptide chain comprising, in N- to C-terminal order:
      (i) a sixth amino acid sequence having at least about 95% sequence identity to any one of SEQ ID NOs: 346, 347, 348, 349, 350, and 351;
      (ii) a non-cleavable linker/non-cleavable means for connecting the sixth amino acid sequence to an eighth amino sequence, optionally wherein the linker/means comprises or consists of a seventh amino acid sequence comprising an amino acid sequence set forth in Table E;
      (iii) the eighth amino acid sequence, having at least about 95% sequence identity to any one of SEQ ID NOs: 3, 4, and 5;
      (iv) a ninth amino acid sequence comprising one or more amino acid sequences set forth in Table B or Table D; and
      (v) a tenth amino acid sequence having at least about 95% sequence identity to the amino acid sequence of SEQ ID NO: 2.
69. The IL2 proprotein of embodiment 68, wherein the first polypeptide comprises, N-terminal to the first amino acid sequence, an eleventh amino acid sequence having at least about 95% sequence identity to any one of SEQ ID NOs: 352, 353, 354, and 355.
70. The IL2 proprotein of embodiment 69, wherein the eleventh amino acid sequence is the amino acid sequence of SEQ ID NO: 352.
71. The IL2 proprotein of embodiment 69, wherein the eleventh amino acid sequence is the amino acid sequence of SEQ ID NO: 353.
72. The IL2 proprotein of embodiment 69, wherein the eleventh amino acid sequence is the amino acid sequence of SEQ ID NO: 354.
73. The IL2 proprotein of embodiment 69, wherein the eleventh amino acid sequence is the amino acid sequence of SEQ ID NO: 355.
74. The IL2 proprotein of any one of embodiments 68 to 73, wherein the second polypeptide comprises, N-terminal to the first amino acid sequence, a twelfth amino acid sequence having at least about 95% sequence identity to any one of SEQ ID NOs: 352, 353, 354, and 355.
75. The IL2 proprotein of embodiment 74, wherein the twelfth amino acid sequence is the amino acid sequence of SEQ ID NO: 352.
76. The IL2 proprotein of embodiment 74, wherein the twelfth amino acid sequence is the amino acid sequence of SEQ ID NO: 353.
77. The IL2 proprotein of embodiment 74, wherein the twelfth amino acid sequence is the amino acid sequence of SEQ ID NO: 354.
78. The IL2 proprotein of embodiment 74, wherein the twelfth amino acid sequence is the amino acid sequence of SEQ ID NO: 355.
79. The IL2 proprotein of any one of embodiments 68 to 78, wherein the first amino acid sequence has at least about 98% sequence identity to any one of SEQ ID NOs: 346, 347, 348, 349, 350, and 351.
80. The IL2 proprotein of any one of embodiments 68 to 78, wherein the first amino acid sequence is the amino acid sequence of any one of SEQ ID NOs: 346, 347, 348, 349, 350, and 351.
81. The IL2 proprotein of embodiment 80, wherein the first amino acid sequence is the amino acid sequence of SEQ ID NO:350.
82. The IL2 proprotein of embodiment 80, wherein the first amino acid sequence is the amino acid sequence of SEQ ID NO:351.
83. The IL2 proprotein of any one of embodiments 68 to 82, wherein the first amino acid sequence is 350 or fewer amino acids in length.
84. The IL2 proprotein of any one of embodiments 68 to 82, wherein the first amino acid sequence is 330 or fewer amino acids in length.
85. The IL2 proprotein of any one of embodiments 68 to 84, wherein the sixth amino acid sequence has at least about 98% sequence identity to any one of SEQ ID NOs: 346, 347, 348, 349, 350, and 351.
86. The IL2 proprotein of any one of embodiments 68 to 84, wherein the sixth amino acid sequence is the amino acid sequence of any one of SEQ ID NOs: 346, 347, 348, 349, 350, and 351.
87. The IL2 proprotein of embodiment 86, wherein the sixth amino acid sequence is the amino acid sequence of SEQ ID NO:350.
88. The IL2 proprotein of embodiment 86, wherein the sixth amino acid sequence is the amino acid sequence of SEQ ID NO:351.
89. The IL2 proprotein of any one of embodiments 68 to 88, wherein the sixth amino acid sequence is 350 or fewer amino acids in length.
90. The IL2 proprotein of any one of embodiments 68 to 88, wherein the sixth amino acid sequence is 330 or fewer amino acids in length.
91. The IL2 proprotein of any one of embodiments 68 to 90, wherein the second amino acid sequence is an amino acid sequence set forth in Table E.
92. The IL2 proprotein of embodiment 91, wherein the second amino acid sequence is the amino acid sequence (GGGGS)ₙ, wherein n is 1, 2, 3, 4, or 5.
93. The IL2 proprotein of any one of embodiments 68 to 92, wherein the second amino acid sequence is 25 or fewer amino acids in length.
94. The IL2 proprotein of any one of embodiments 68 to 92, wherein the second amino acid sequence is 15 or fewer amino acids in length.
95. The IL2 proprotein of any one of embodiments 68 to 92, wherein the second amino acid sequence is 6 or fewer amino acids in length.
96. The IL2 proprotein of any one of embodiments 68 to 95, wherein the seventh amino acid sequence is an amino acid sequence set forth in Table E.
97. The IL2 proprotein of embodiment 96, wherein the seventh amino acid sequence is the amino acid sequence (GGGGS)ₙ, wherein n is 1, 2, 3, 4, or 5.
98. The IL2 proprotein of any one of embodiments 68 to 97, wherein the seventh amino acid sequence is 25 or fewer amino acids in length.
99. The IL2 proprotein of any one of embodiments 68 to 97, wherein the seventh amino acid sequence is 15 or fewer amino acids in length.
100. The IL2 proprotein of any one of embodiments 68 to 97, wherein the seventh amino acid sequence is 6 or fewer amino acids in length.
101. The IL2 proprotein of any one of embodiments 68 to 100, wherein the third amino acid sequence has at least about 98% sequence identity to any one of SEQ ID NOs: 3, 4, and 5.
102. The IL2 proprotein of any one of embodiments 68 to 100, wherein the third amino acid sequence is the amino acid sequence of any one of SEQ ID NOs: 3, 4, and 5.
103. The IL2 proprotein of embodiment 102, wherein the third amino acid sequence is the amino acid sequence of SEQ ID NO:3.
104. The IL2 proprotein of embodiment 102, wherein the third amino acid sequence is the amino acid sequence of SEQ ID NO:4.
105. The IL2 proprotein of embodiment 102, wherein the third amino acid sequence is the amino acid sequence of SEQ ID NO:5.
106. The IL2 proprotein of any one of embodiments 68 to 105, wherein the third amino acid sequence is 255 or fewer amino acids in length.
107. The IL2 proprotein of any one of embodiments 68 to 105, wherein the third amino acid sequence is 225 or fewer amino acids in length.
108. The IL2 proprotein of any one of embodiments 68 to 105, wherein the third amino acid sequence is 170 or fewer amino acids in length.
109. The IL2 proprotein of any one of embodiments 68 to 108, wherein the eighth amino acid sequence has at least about 98% sequence identity to any one of SEQ ID NOs: 3, 4, and 5.
110. The IL2 proprotein of any one of embodiments 68 to 109, wherein the eighth amino acid sequence is the amino acid sequence of any one of SEQ ID NOs: 3, 4, and 5.
111. The IL2 proprotein of embodiment 110, wherein the eighth amino acid sequence is the amino acid sequence of SEQ ID NO:3.
112. The IL2 proprotein of embodiment 110, wherein the eighth amino acid sequence is the amino acid sequence of SEQ ID NO:4.
113. The IL2 proprotein of embodiment 110, wherein the eighth amino acid sequence is the amino acid sequence of SEQ ID NO:5.
114. The IL2 proprotein of any one of embodiments 68 to 113, wherein the eighth amino acid sequence is 255 or fewer amino acids in length.
115. The IL2 proprotein of any one of embodiments 68 to 113, wherein the eighth amino acid sequence is 225 or fewer amino acids in length.
116. The IL2 proprotein of any one of embodiments 68 to 113, wherein the eighth amino acid sequence is 170 or fewer amino acids in length.
117. The IL2 proprotein of any one of embodiments 68 to 116, wherein the fourth amino acid sequence comprises one or more amino acid sequences set forth in Table B.
118. The IL2 proprotein of embodiment 117, wherein the fourth amino acid sequence comprises the amino acid sequence HPVGLLAR (SEQ ID NO: 261).
119. The IL2 proprotein of embodiment 117, wherein the fourth amino acid sequence comprises the amino acid sequence VPLSLYSG (SEQ ID NO: 149).
120. The IL2 proprotein of embodiment 117, wherein the fourth amino acid sequence comprises the amino acid sequence ISSGLLS (SEQ ID NO: 64).
121. The IL2 proprotein of embodiment 117, wherein the fourth amino acid sequence comprises the amino acid sequence PLGLWSQ (SEQ ID NO: 105).
122. The IL2 proprotein of any one of embodiments 68 to 116, wherein the fourth amino acid sequence is an amino acid sequence set forth in Table D.
123. The IL2 proprotein of embodiment 122, wherein the fourth amino acid sequence is the amino acid sequence GGGISSGLLSGRSDNHGGGISSGLLSGRSDNHGGS (SEQ ID NO: 185).
124. The IL2 proprotein of embodiment 122, wherein the fourth amino acid sequence is the amino acid sequence GGSHPVGLLARGGGHPVGLLARGGGHPVGLLARGS (SEQ ID NO: 189).
125. The IL2 proprotein of embodiment 122, wherein the fourth amino acid sequence is the amino acid sequence GGGHPVGLLARGGGS (SEQ ID NO: 339).
126. The IL2 proprotein of embodiment 122, wherein the fourth amino acid sequence is the amino acid sequence GGGISSGLLSGRSDNHGGGS (SEQ ID NO: 338).
127. The IL2 proprotein of embodiment 122, wherein the fourth amino acid sequence is the amino acid sequence GGGGSGGGGSGGGGSVPLSLYSGGGSGGSGGSGS (SEQ ID NO: 207).
128. The IL2 proprotein of any one of embodiments 68 to 127, wherein the ninth amino acid sequence comprises one or more amino acid sequences set forth in Table B.
129. The IL2 proprotein of embodiment 128, wherein the ninth amino acid sequence comprises the amino acid sequence HPVGLLAR (SEQ ID NO: 261).
130. The IL2 proprotein of embodiment 128, wherein the ninth amino acid sequence comprises the amino acid sequence VPLSLYSG (SEQ ID NO: 149).
131. The IL2 proprotein of embodiment 128, wherein the ninth amino acid sequence comprises the amino acid sequence ISSGLLS (SEQ ID NO: 64).
132. The IL2 proprotein of embodiment 128, wherein the ninth amino acid sequence comprises the amino acid sequence PLGLWSQ (SEQ ID NO: 105).
133. The IL2 proprotein of any one of embodiments 68 to 127, wherein the ninth amino acid sequence is an amino acid sequence set forth in Table D.
134. The IL2 proprotein of embodiment 133, wherein the ninth amino acid sequence is the amino acid sequence GGGISSGLLSGRSDNHGGGISSGLLSGRSDNHGGS (SEQ ID NO: 185).
135. The IL2 proprotein of embodiment 133, wherein the ninth amino acid sequence is the amino acid sequence GGSHPVGLLARGGGHPVGLLARGGGHPVGLLARGS (SEQ ID NO: 189).
136. The IL2 proprotein of embodiment 133, wherein the ninth amino acid sequence is the amino acid sequence GGGHPVGLLARGGGS (SEQ ID NO: 339).
137. The IL2 proprotein of embodiment 133, wherein the ninth amino acid sequence is the amino acid sequence GGGISSGLLSGRSDNHGGGS (SEQ ID NO: 338).
138. The IL2 proprotein of embodiment 133, wherein the ninth amino acid sequence is the amino acid sequence GGGGSGGGGSGGGGSVPLSLYSGGGSGGSGGSGS (SEQ ID NO: 207).
139. The IL2 proprotein of any one of embodiments 68 to 138, wherein the fifth amino acid sequence has at least about 98% sequence identity to SEQ ID NO:2.
140. The IL2 proprotein of any one of embodiments 68 to 138, wherein the fifth amino acid sequence is the amino acid sequence of SEQ ID NO:2.
141. The IL2 proprotein of any one of embodiments 68 to 140, wherein the fifth amino acid sequence is 150 or fewer amino acids in length.
142. The IL2 proprotein of any one of embodiments 68 to 140, wherein the fifth amino acid sequence is 135 or fewer amino acids in length.
143. The IL2 proprotein of any one of embodiments 68 to 142, wherein the tenth amino acid sequence has at least about 98% sequence identity to SEQ ID NO:2.
144. The IL2 proprotein of any one of embodiments 68 to 142, wherein the tenth amino acid sequence is the amino acid sequence of SEQ ID NO:2.
145. The IL2 proprotein of any one of embodiments 68 to 144, wherein the tenth amino acid sequence is 150 or fewer amino acids in length.
146. The IL2 proprotein of any one of embodiments 68 to 144, wherein the tenth amino acid sequence is 135 or fewer amino acids in length.
147. The IL2 proprotein of any one of embodiments 68 to 146, wherein the first polypeptide chain lacks additional sequences C-terminal to the first amino acid sequence.
148. The IL2 proprotein of any one of embodiments 68 to 147, wherein the first polypeptide chain lacks additional sequences between the first and second amino acid sequences.
149. The IL2 proprotein of any one of embodiments 68 to 148, wherein the first polypeptide chain lacks additional sequences between the second and third amino acid sequences.
150. The IL2 proprotein of any one of embodiments 68 to 149, wherein the first polypeptide chain lacks additional sequences between the third and fourth amino acid sequences.
151. The IL2 proprotein of any one of embodiments 68 to 150, wherein the first polypeptide chain lacks additional sequences between the fourth and fifth amino acid sequences.
152. The IL2 proprotein of any one of embodiments 68 to 151, wherein the second polypeptide chain lacks additional sequences C-terminal to the sixth amino acid sequence.
153. The IL2 proprotein of any one of embodiments 68 to 152, wherein the second polypeptide chain lacks additional sequences between the sixth and seventh amino acid sequences.
154. The IL2 proprotein of any one of embodiments 68 to 153, wherein the second polypeptide chain lacks additional sequences between the seventh and eighth amino acid sequences.
155. The IL2 proprotein of any one of embodiments 68 to 154, wherein the second polypeptide chain lacks additional sequences between the eighth and ninth amino acid sequences.
156. The IL2 proprotein of any one of embodiments 68 to 155, wherein the second polypeptide chain lacks additional sequences between the ninth and tenth amino acid sequences.
157. The IL2 proprotein of any one of embodiments 68 to 156, wherein the first polypeptide and the second polypeptide are identical.
158. A nucleic acid or plurality of nucleic acids encoding the IL2 proprotein of any one of embodiments 1 to 157.
159. A host cell engineered to express the IL2 proprotein of any one of embodiments 1 to 157 or the nucleic acid(s) of embodiment 64.
160. A method of producing the IL2 proprotein of any one of embodiments 1 to 157, comprising culturing the host cell of embodiment 159 and recovering the IL2 proprotein expressed thereby.
161. A pharmaceutical composition comprising the IL2 proprotein of any one of embodiments 1 to 157 and an excipient.
162. A method of treating cancer, comprising administering to a subject in need thereof the IL2 proprotein of any one of embodiments 1 to 157 or the pharmaceutical composition of embodiment 161.
163. The method of embodiment 162, wherein the IL2 proprotein comprises at least one targeting moiety that is capable of binding to a target molecule and wherein the cancer is associated with expression of the target molecule, e.g., a TAA and associated cancer as set forth in Table I.
164. The method of embodiment 163, wherein an activated IL2 protein comprising the IL2 moiety is produced by cleavage of one or more protease-cleavable linkers in the IL2 proprotein by one or more proteases expressed by the cancer tissue.
165. The method of embodiment 164, wherein the IL2 protein is selectively activated in the cancer tissue.
166. A method of localized delivery of an IL2 protein, comprising administering to a subject an IL2 proprotein according to any one of embodiments 1 to 157 (or a pharmaceutical composition comprising the IL2 proprotein and an excipient) which has one or more protease-cleavable linkers, each comprising one or more substrates for one or more proteases expressed by a tissue to which the IL2 protein is to be locally delivered.
167. The method of embodiment 166, wherein the IL2 proprotein comprises one or more targeting moieties that recognize a target molecule expressed by the tissue.
168. The method of embodiment 167, wherein the IL2 proprotein comprises two targeting moieties that each recognize a target molecule expressed by the tissue.
169. The method of embodiment 167 or embodiment 168, wherein the tissue is cancer tissue.
170. The method of embodiment 169, wherein the one or more targeting moieties are capable of binding to an extracellular matrix (“ECM”) antigen, a tumor reactive lymphocyte antigen, a cell surface molecule of tumor or viral lymphocytes, a T-cell antigen (“TCA”), a checkpoint inhibitor, or a tumor-associated antigen (“TAA”).
171. The method of any one of embodiments 166 to 170, wherein an activated IL2 protein comprising the IL2 moiety is produced by cleavage of one or more protease-cleavable linkers in the IL2 proprotein by one or more proteases in the tissue.
172. A method of treating cancer with an IL2 protein that is selectively activated in cancer tissue, comprising administering to a subject in need thereof an IL2 proprotein according to any one of embodiments 1 to 157 (or a pharmaceutical composition comprising the IL2 proprotein and an excipient) which has one or more protease-cleavable linkers, each comprising one or more substrates for one or more proteases expressed by cancer tissue to which the IL2 protein.
173. The method of embodiment 172, wherein the IL2 proprotein comprises one or more targeting moieties that recognize a target molecule expressed by the cancer tissue.
174. The method of embodiment 173, wherein the IL2 proprotein comprises two targeting moieties that each recognize a target molecule expressed by the cancer tissue.
175. The method of embodiment 173 or embodiment 174, wherein the one or more targeting moieties are capable of binding to an extracellular matrix (“ECM”) antigen, a tumor reactive lymphocyte antigen, a cell surface molecule of tumor or viral lymphocytes, a T-cell antigen (“TCA”), a checkpoint inhibitor, or a tumor-associated antigen (“TAA”).
176. The method of any one of embodiments 172 to 175, wherein an activated IL2 protein comprising the IL2 moiety is produced by cleavage of one or more protease-cleavable linkers in the IL2 proprotein by one or more proteases in the cancer tissue.
177. A method of administering to the subject IL2 therapy with reduced systemic exposure and/or reduced systemic toxicity, comprising administering to a subject the IL2 therapy in the form of an IL2 proprotein according to any one of embodiments 1 to 157 (or a pharmaceutical composition comprising the IL2 proprotein and an excipient) which has one or more protease-cleavable linkers, each comprising one or more substrates for one or more proteases expressed by a tissue for which IL2 therapy is desirable and/or intended.
178. The method of embodiment 177, wherein the IL2 proprotein comprises one or more targeting moieties that recognize a target molecule expressed by the tissue.
179. The method of embodiment 178, wherein the IL2 proprotein comprises two targeting moieties that each recognize a target molecule expressed by the tissue.
180. The method of any one of embodiments 177 to 179, wherein the tissue is cancer tissue.
181. The method of embodiment 180, wherein the one or more targeting moieties are capable of binding to an extracellular matrix (“ECM”) antigen, a tumor reactive lymphocyte antigen, a cell surface molecule of tumor or viral lymphocytes, a T-cell antigen (“TCA”), a checkpoint inhibitor, or a tumor-associated antigen (“TAA”).
182. The method of any one of embodiments 177 to 181, wherein an activated IL2 protein comprising the IL2 moiety is produced by cleavage of one or more protease-cleavable linkers in the IL2 proprotein by one or more proteases in the tissue.
183. A method of treating cancer with an IL2 protein that is selectively activated in cancer tissue, comprising administering to a subject in need thereof an IL2 proprotein according to any one of embodiments 1 to 157 (or a pharmaceutical composition comprising the IL2 proprotein and an excipient) which has one or more protease-cleavable linkers, each comprising one or more substrates for one or more proteases expressed by the cancer tissue.
184. The method of embodiment 183, wherein the IL2 proprotein comprises one or more targeting moieties that recognize a target molecule expressed by the cancer tissue.
185. The method of embodiment 184, wherein the IL2 proprotein comprises two targeting moieties that each recognize a target molecule expressed by the cancer tissue.
186. The method of embodiment 184 or embodiment 185, wherein the one or more targeting moieties are capable of binding to an extracellular matrix (“ECM”) antigen, a tumor reactive lymphocyte antigen, a cell surface molecule of tumor or viral lymphocytes, a T-cell antigen (“TCA”), a checkpoint inhibitor, or a tumor-associated antigen (“TAA”).
187. The method of any one of embodiments 183 to 186, wherein an activated IL2 protein comprising the IL2 moiety is produced by cleavage of one or more protease-cleavable linkers in the IL2 proprotein by one or more proteases in the cancer tissue.
188. A method of targeted delivery of an activated IL2 protein to cancer tissue, comprising administering to a subject an IL2 proprotein according to any one of embodiments 1 to 157 (or a pharmaceutical composition comprising the IL2 proprotein and an excipient), wherein the IL2 proprotein:
   (a) comprises one or more targeting moieties that recognize a target molecule expressed by the cancer tissue; and
   (b) has one or more protease-cleavable linkers, each comprising one or more substrates for one or more proteases expressed by a tissue for which IL2 therapy is desirable and/or intended.
189. The method of embodiment 188, wherein the IL2 proprotein comprises two targeting moieties that each recognize a target molecule expressed by the cancer tissue.
190. The method of embodiment 188 or embodiment 189, wherein the one or more targeting moieties are capable of binding to an extracellular matrix (“ECM”) antigen, a tumor reactive lymphocyte antigen, a cell surface molecule of tumor or viral lymphocytes, a T-cell antigen (“TCA”), a checkpoint inhibitor, or a tumor-associated antigen (“TAA”).
191. The method of any one of embodiments 188 to 190, wherein an activated IL2 protein comprising the IL2 moiety is produced by cleavage of one or more protease-cleavable linkers in the IL2 proprotein by one or more proteases in the cancer tissue.
192. A method of locally inducing an immune response in a target tissue, comprising administering to a subject an IL2 proprotein according to any one of embodiments 1 to 157 (or a pharmaceutical composition comprising the IL2 proprotein and an excipient) which has one or more targeting moieties capable of binding a target molecule expressed in the target tissue and one or more protease-cleavable linkers, each comprising one or more substrates for one or more proteases expressed in the target tissue.
193. The method of embodiment 192, wherein the IL2 proprotein comprises two targeting moieties that each recognize a target molecule expressed in the target tissue.
194. The method of embodiment 192 or embodiment 98, wherein the target tissue is cancer tissue.
195. The method of any one of embodiments 192 to 194, wherein the one or more targeting moieties are capable of binding to an extracellular matrix (“ECM”) antigen, a tumor reactive lymphocyte antigen, a cell surface molecule of tumor or viral lymphocytes, a T-cell antigen (“TCA”), a checkpoint inhibitor, or a tumor-associated antigen (“TAA”).
196. The method of any one of embodiments 192 to 195, wherein an activated IL2 protein comprising the IL2 moiety is produced by cleavage of one or more protease-cleavable linkers in the IL2 proprotein by one or more proteases in the target tissue.
197. The method of embodiment 196, wherein the IL2 protein induces the immune response against at least one cell type in the target tissue.
198. The method of any one of embodiments 162 to 197, wherein the administration is non-local.
199. The method of embodiment 198, wherein the administration is systemic.
200. The method of embodiment 198, wherein the administration is subcutaneous.

### 8.1. EXAMPLES

### 8.1.1. Example 1: Digestion of Protease-Cleavable Linkers Using Recombinant Proteases

A variety of IL2 proprotein constructs were generated in the format of Targeting Moiety—IL2Rα Moiety—Linker 2—IL2 Moiety. An overview of the IL2 proproteins encoded by the generated constructs is provided in Table 1, below. Table 1 describes a single half antibody of each IL2 proprotein, where each IL2 proprotein comprises two, identical half antibodies.

TABLE 1
IL2 Proproteins

| # | Structure |
|---|---|
| 1 | Fab - Fc(IgG4s)-3xG4S-hIL2Rα(22-186)-5XG4S-hIL2 |
| 2 | Fab - Fc(IgG4s)-3xG4S-hIL2Rα(22-186)-PCL(36AA)-hIL2 |
| 3 | Fab - Fc(IgG4s)-3xG4S-hIL2Rα(22-186)-PCL(36AA)-hIL2 |
| 4 | Fab - Fc(IgG4s)-3xG4S-hIL2Rα(22-186)-PCL(36AA)-hIL2 |
| 5 | Fab - Fc(IgG4s)-3xG4S-hIL2Rα(22-186)-PCL(36AA)-hIL2 |
| 6 | Fab - Fc(IgG4s)-3xG4S-hIL2Rα(22-186)-PCL(36AA)-hIL2 |
| 7 | Fab - Fc(IgG4s)-3xG4S-hIL2Rα(22-186)-PCL(36AA)-hIL2 |

The term “Targeting Moiety” in reference to an experimental construct is a shorthand reference to an antibody with Fc domains and N-terminal Fab domains and a non-cleavable linker (having amino acid sequence GGGGSGGGGSGGGGS (SEQ ID NO: 157)) C-terminal to the Fc domains. The IL2Rα moiety has the amino acid sequence ELCDDDPPEIPHATFKAMAYKEGTMLNCECKRGFRRIKSGSLYMLCTGNSSHSSWDNQCQ CTSSATRNTTKQVTPQPEEQKERKTTEMQSPMQPVDQASLPGHCREPPPWENEATERIY HFVVGQMVYYQCVQGYRALHRGPAESVCKMTHGKTRWTQPQLICTG (SEQ ID NO: 4). The IL2 moiety has the amino acid sequence APTSSSTKKTQLQLEHLLLDLQMILNGINNYKNPKLTRMLTFKFYMPKKATELKHLQCLEEEL KPLEEVLNLAQSKNFHLRPRDLISNINVIVLELKGSETTFMCEYADETATIVEFLNRWITFCQS IISTLT (SEQ ID NO: 2). The various constructs include the protease-cleavable linkers PCL1, PCL2, PCL3, PCL4, PCL5 and PCL6 and the non-cleavable linkers NCL1 and NCL2 between the IL2Rα moiety and the IL2Rα.

To test if several protease-cleavable linkers in the Targeting Moiety—IL2Rα Moiety—Linker—IL2 Moiety were accessible for digestion by recombinant proteases, the above mentioned constructs were digested using different recombinant human proteases such as uPA, MT-SP1, MMP2 and MMP9 (purchased from R&D Systems).

Briefly, 2 μg of each construct (containing PCL1, PCL2, PCL3, PCL4, PCL5, PCL6, NCL1 and NCL2 between IL2Rα and IL2) was digested at 37° C. for 24 hours with or without 50 ng of each of the recombinant protease in the assay buffer recommended by manufacturer. After digestion, SDS sample loading buffer was added to each sample and run on an Invitrogen 4-12% Tris-Glycine gel for SDS PAGE analysis. Different assay buffer was used for each protease:
(a) With no enzyme in 100 μl of assay buffer 50 mM Tris, 0.01% (v/v) Tween® 20, pH 8.5
(b) With 50 ng of uPA enzyme in 100 μl of assay buffer 50 mM Tris, 0.01% (v/v) Tween® 20, pH 8.5
(c) With 50 ng of Matriptase/ST14 enzyme in 100 μl of assay buffer 50 mM Tris, 50 mM NaCl, 0.01% (v/v) Tween® 20, pH 9.0
(d) With 50 ng of APMA activated MMP2 enzyme in 100 μl of assay buffer 50 mM Tris, 10 mM CaCl₂, 150 mM NaCl, 0.05% (w/v) Brij 35, pH 7.5 (TCNB)
(e) With 50 ng of APMA activated MMP9 enzyme in 100 μl of assay buffer 50 mM Tris, 10 mM CaCl₂, 150 mM NaCl, 0.05% Brij-35 (w/v), pH 7.5 (TCNB)

The gel was stained using SimplyBlue Stain from Invitrogen to visualize the bands.

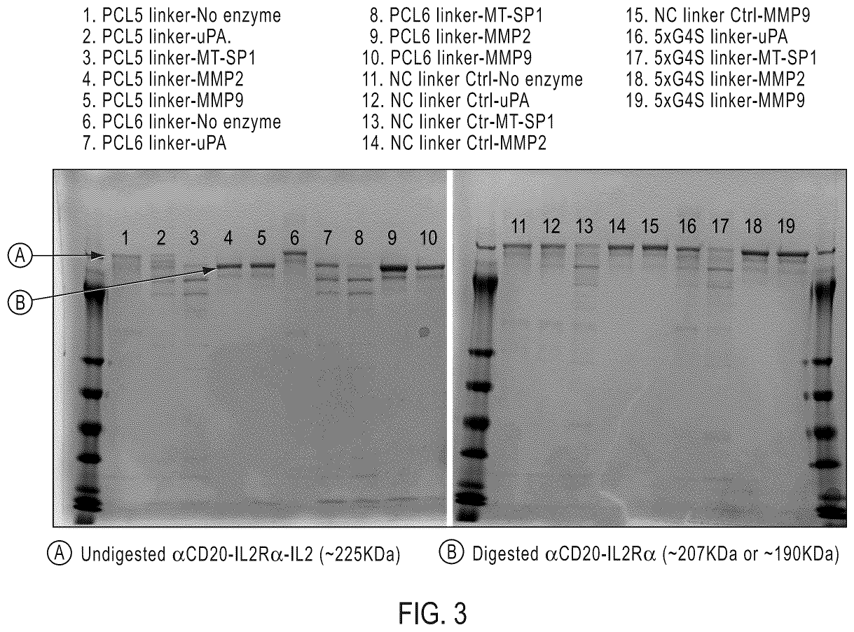

The undigested full-length construct runs at 225 KDa (lane 1 of FIGS. 2 and 3). The correctly digested Targeting Moiety-IL2Rα, with both IL2 cleaved at the linker sequences, runs at ˜200 KDa (lane 2 of FIGS. 2 and 3) suggesting both linker sites are accessible for the proteases to cleave. The majority of the protease substrates tested can be cleaved by multiple proteases that share similar substrate sequence requirement. Some proteases show cleavage outside of the cleavable linker sequence.

### 8.1.2. Example 2: Activity of Protease Released IL2 in a YT/STAT5 Luc Luciferase Assay

To test if the protease released IL2 was active, recombinant protease digested cleavable linker constructs were tested in a YT/STAT5 luc luciferase assay. Briefly, 2 μg of αCD20-IL2Rα-PCL-IL2 was digested for 24 hr at 37 C with 50 ng of Matriptase (R&D, Cat#3946-SEB-010) or uPA (R&D, Cat #1310-SE) in 100 μl of assay buffer 50 mM Tris, 0.01% (v/v) Tween® 20, pH 8.5. YT/pGL452-STAT5 luc/CL4/hIL7Ra cells were incubated with a titration of IL2(WT)-mmh, αCD20-IL2Rα-G4S-IL2 and MT-SP1 or uPA digested and undigested CD20-IL2Rα-PCL-IL2 at 37 C, 5% CO₂ for 4-6 hours. After incubation, the plate and One-Glow luciferase substrate were equilibrated at room temperature. 100 μl of One-Glo substrate was added per well and incubated for 3-5 minutes. Luminescence was measured on Envision.

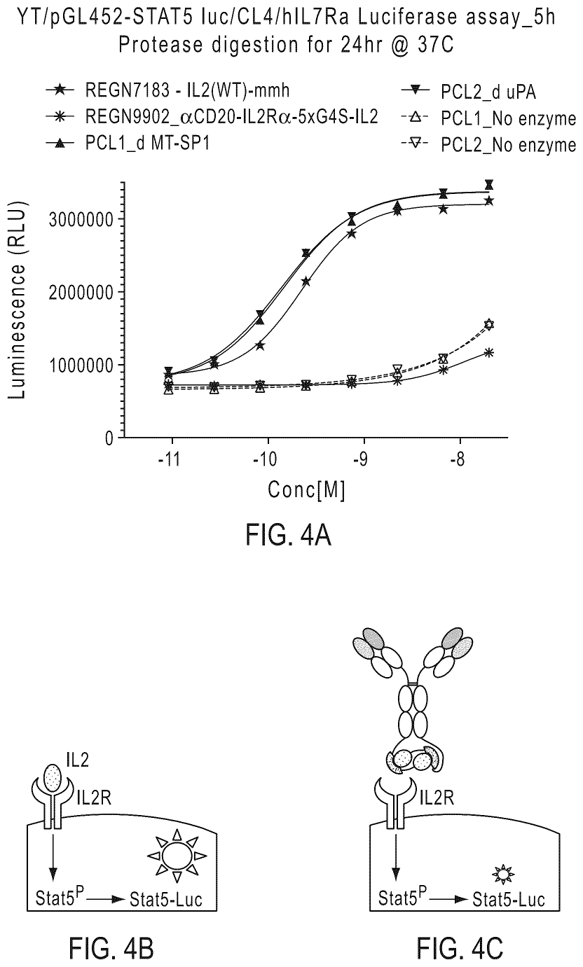

Referring to FIG. 4, protease mediated cleavage of IL2 from CD20-IL2Rα-PCL-IL2 leads to improved activity in YT/STAT5 luc/CL4 luciferase assay. The IL2 in Targeting Moiety-IL2Rα-IL2 format exists in an auto-inhibited transdimer format with minimal luciferase activity as shown by the CD20-IL2Rα-G4S-IL2. Upon release, after MT-SP1 or uPA digestion, the cleaved IL2 has STAT5 luc activity similar to IL2(WT)-mmh suggesting that protease-cleaved IL2 is fully bioactive

### 8.1.3. Example 3: In-Vivo Efficacy of Tumor Targeted IL2Rα-IL2 with Protease-Cleavable Linker

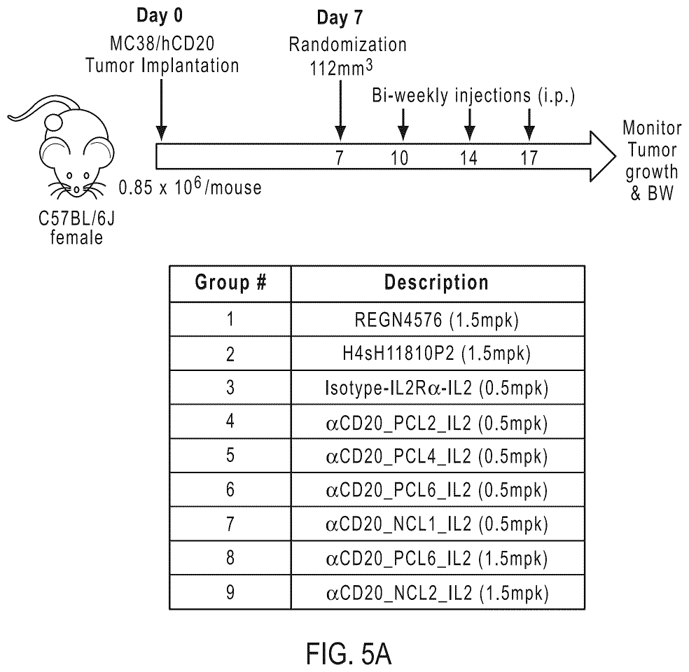

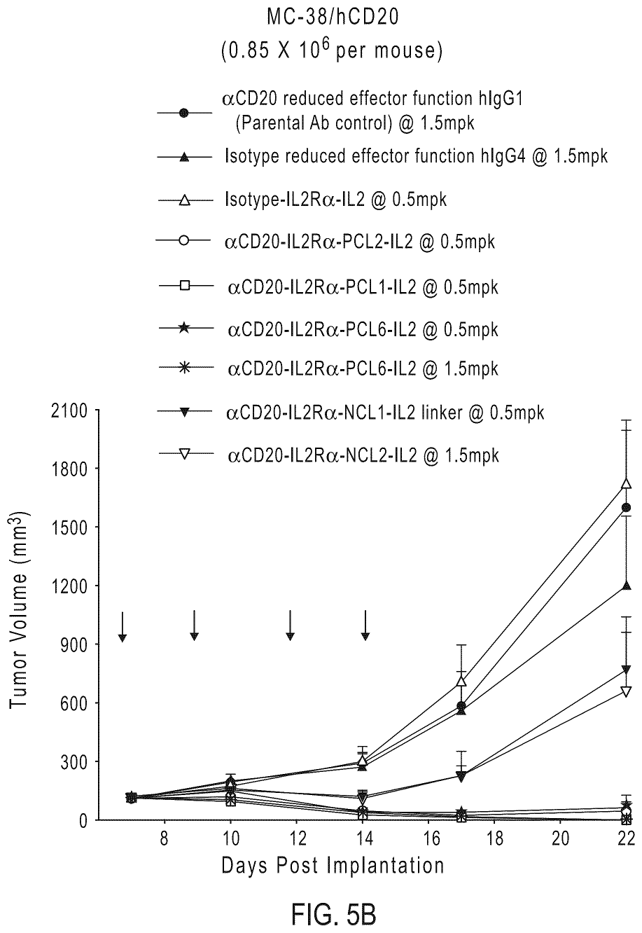

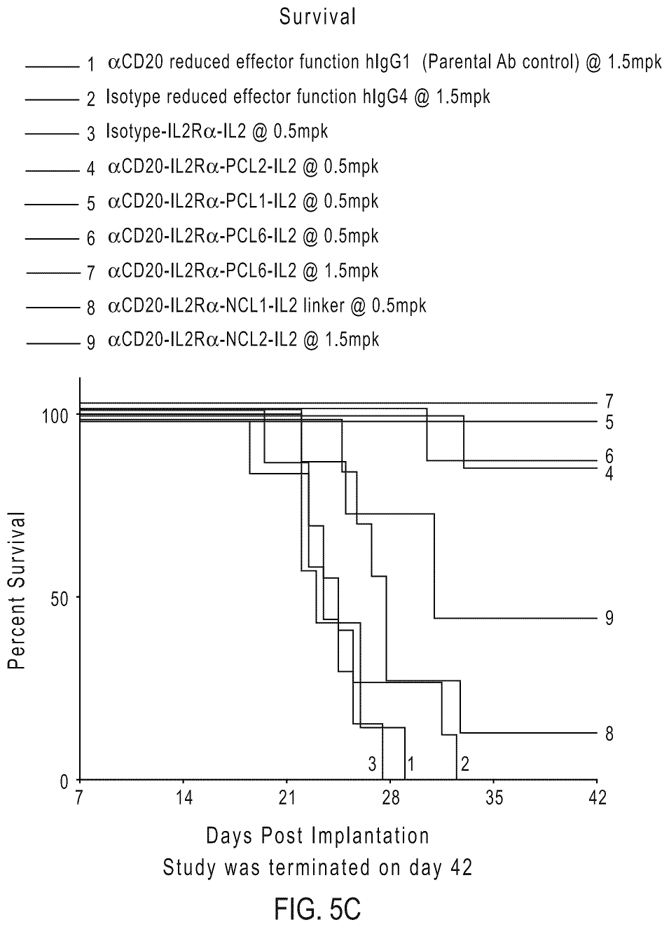

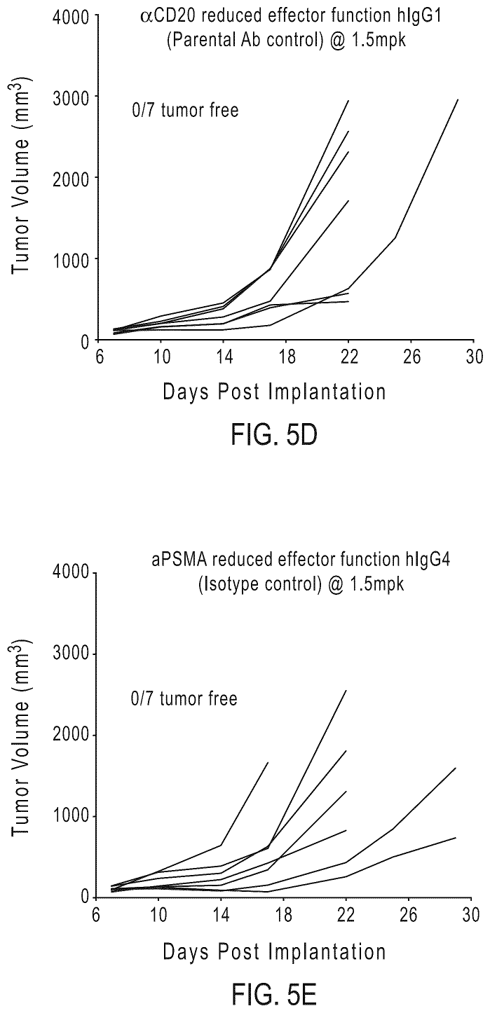

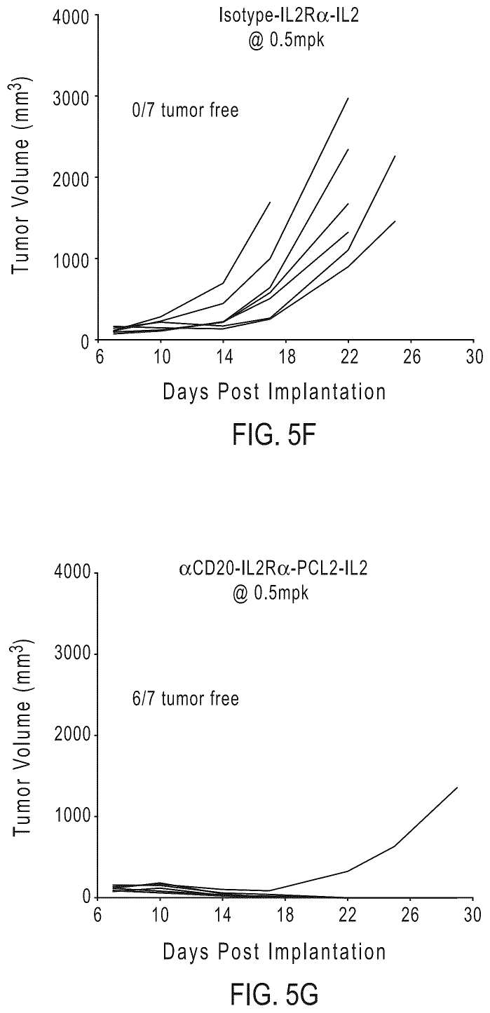

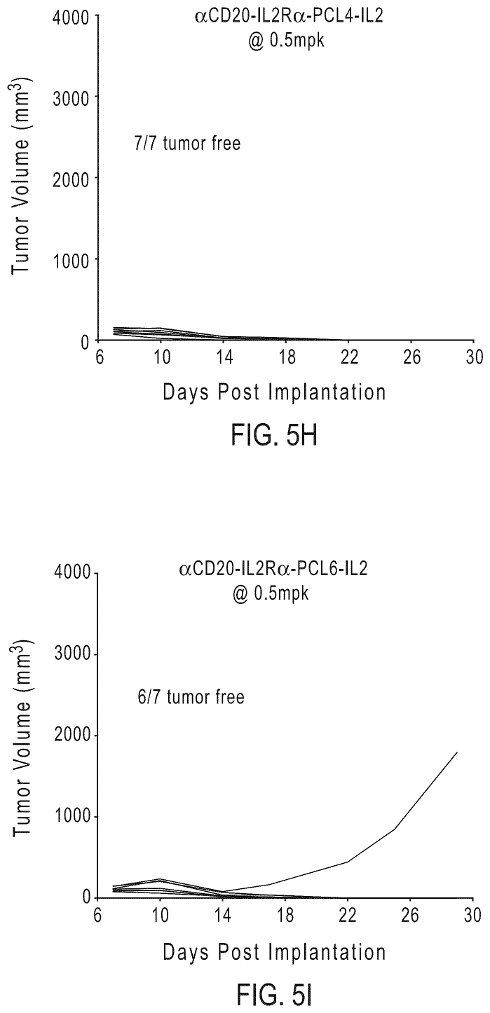

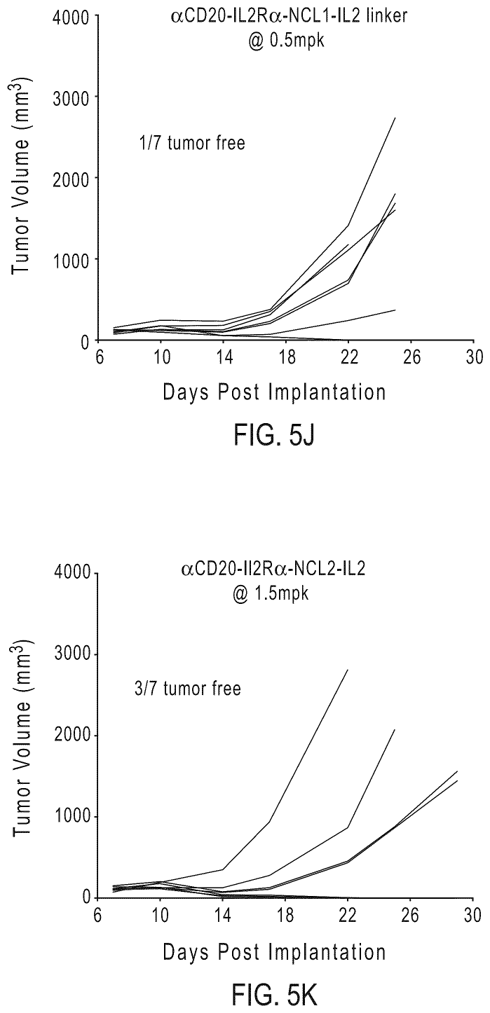

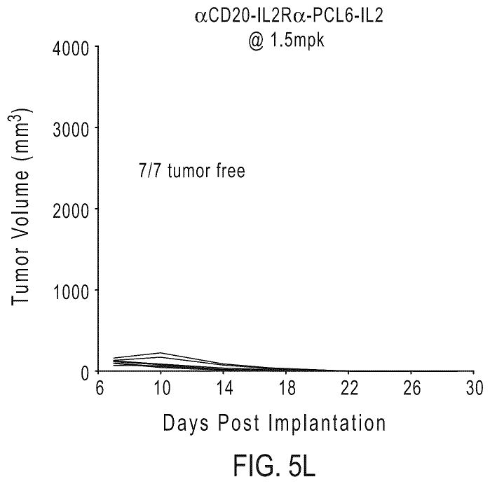

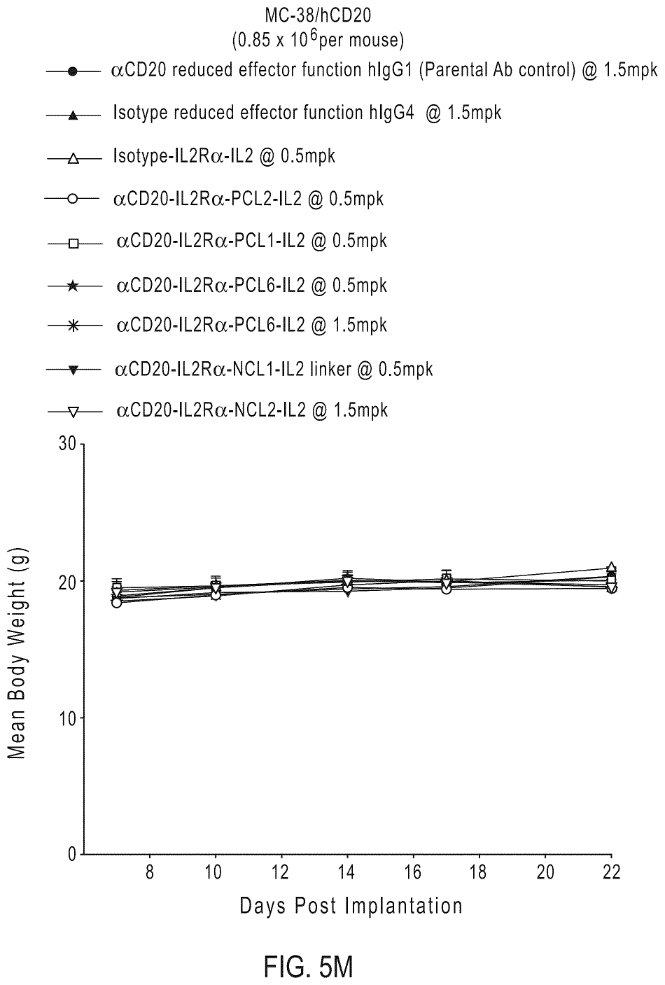

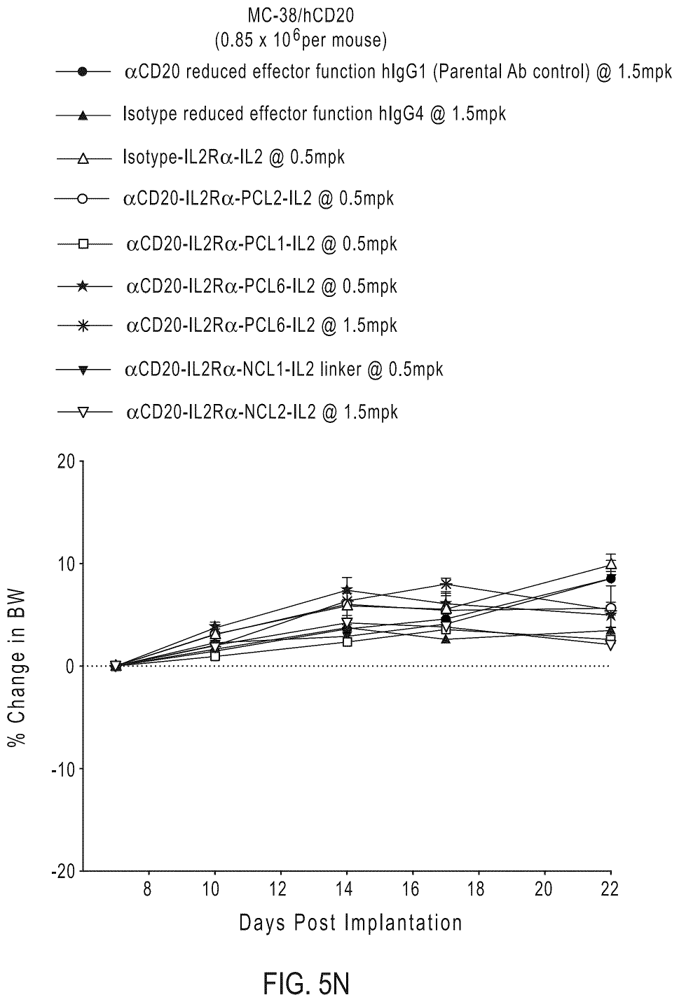

The schematic of FIG. 5A shows that 8-10-week-old female C57BL/6 mice (The Jackson Laboratory) were implanted with 0.85×10⁶ MC38/hCD20 tumor cells subcutaneously into the right hind flank. Mice were randomized when tumor size reached ˜110 mm³ and were treated intraperitoneally with different proteins. Treatment was administered intraperitoneally biweekly, for a total of 4 doses. Tumors were measured semiweekly using a digital caliper and the tumor sizes were calculated as length×width²/2. Measurements were performed until the tumor size reached 2250 mm³, or until any mouse in any group needed to be euthanized due to ulceration. Body weight changes were monitored over the course of the study. Observations were extended to Day 42

FIGS. 5B-5N show the anti-tumor efficacy of TAA-targeted IL2Rα-IL2 with or without cleavable linker. The αCD20 targeted constructs with cleavable linkers showed significantly better anti-tumor efficacy and tumor free survival until the study was terminated compared to CD20 targeted construct with non-cleavable linker at the doses tested in a MC38/hCD20 mouse model. The bivalent αCD20 with effectorless Fc and non-targeted isotype groups showed no tumor growth control. As shown in FIGS. 5M and 5N, there was no significant change in body weight in any of the groups of treated mice and the mice did not show any weight loss upon treatment. FIGS. 5D-5L shows the individual tumor curves for all the groups tested in the study.

### 8.1.4. Example 4: Stability of Protease-Cleavable Linker In-Vivo

As illustrated in FIG. 6A, 8-10-week-old non-tumor bearing female C57BL/6 mice (The Jackson Laboratory) were dosed with a single 5 mpk subcutaneous injection of αCD20-IL2Rα-linker-IL2 proteins with 3 different cleavable linkers and the 2 non-cleavable linker controls (n=2 mice per group). Plasma was collected from mice at 16 hr, 40 hr and 64 hours post treatment to check for stability of dosed proteins in serum by western blot analysis (FIG. 6B). 1 ul serum was run along with undigested and recombinant protease digested CD20-IL2Rα-PCL1-IL2 as control on a 4-12% Tris-Glycine gel followed by western blot using Goat Anti-Human IgG—Fc specific, HRP conjugate (Sigma A0170-1ML) as detect. A short 3-day in-vivo stability study was done in non-tumor bearing mice. The 3 cleavable linkers tested showed a differential cleavage pattern in serum over the course of 3 days. αCD20-IL2Rα-PCL2-IL2 was the most stable at the 3 time points tested—16 hr, 40 hr and 64 hr. CD20-IL2Rα-PCL4-IL2 showed intermediate stability and CD20-IL2Rα-PCL5-IL2 was the least stable and showed digestion of cleavable linker and cleavage of IL2 even at the 16 hr time point. The non-cleavable controls showed minimal cleavage of IL2 as expected. The undigested full-length construct runs at 225 KDa (FIG. 6B, lane 1). The completely digested Targeting Moiety-IL2Rα, with both IL2 cleaved at the linker sequences, runs at ˜200 KDa (FIG. 6B, lane 2).

### 8.1.5. Example 5: Contribution of Tumor Targeting to the Efficacy of αCD20-IL2Rα-cleavable linker-IL2

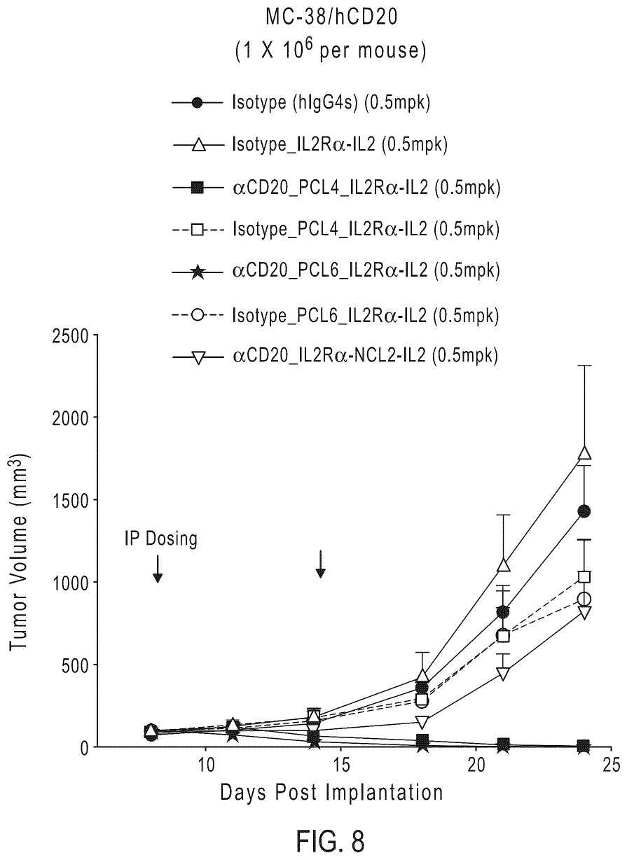

FIG. 7 shows the implantation and treatment protocol in a MC38/hCD20 model 8-10-week-old female C57BL/6 mice (The Jackson Laboratory) implanted with 1×10⁶ MC38/hCD20 tumor cells subcutaneously into the right hind flank. Mice were randomized when tumor size reached ˜110 mm³ and were treated intraperitoneally with the different proteins. Treatment was administered intraperitoneally weekly, for a total of 2 doses. Tumors were measured semiweekly using a digital caliper and the tumor sizes were calculated as length×width²/2 (FIG. 8). Measurements were performed until the tumor size reached 2250 mm³, or until any mouse in any group needed to be euthanized due to ulceration. Body weight changes were monitored over the course of the study.

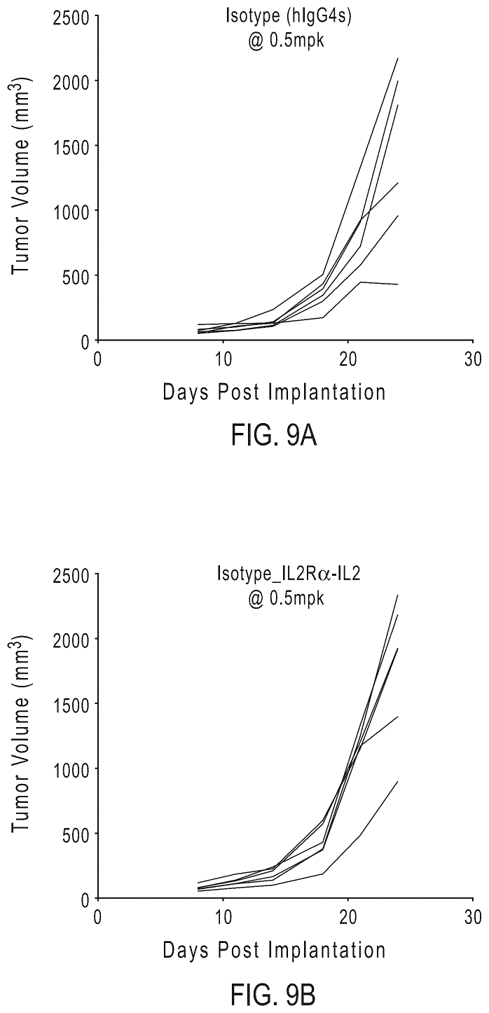

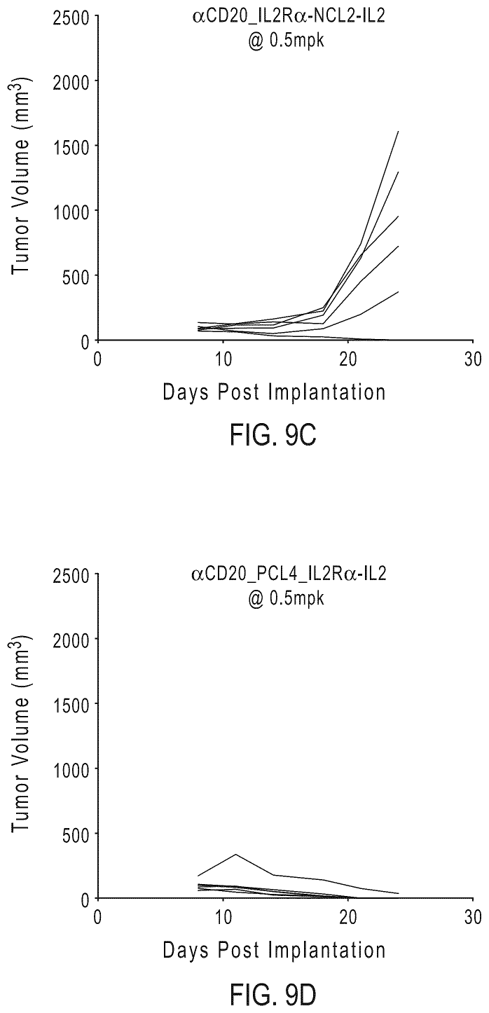

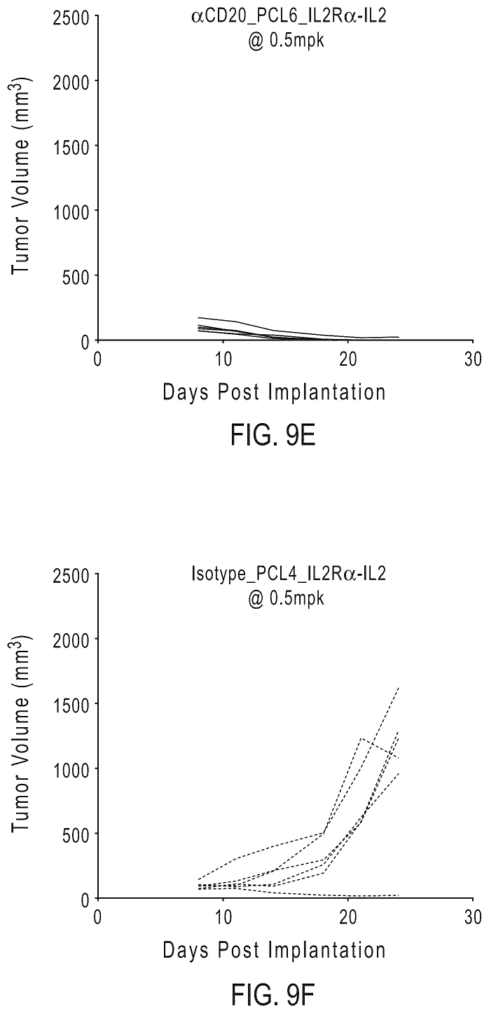

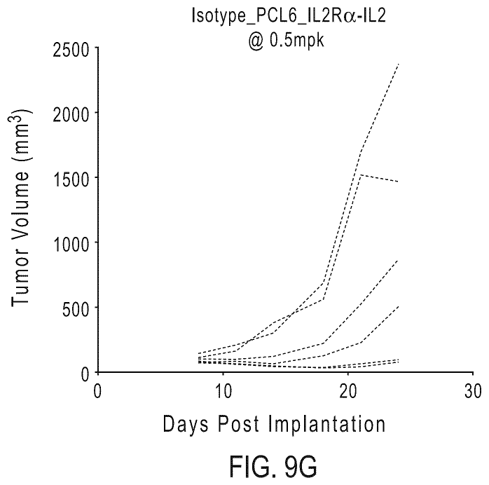

We evaluated the contribution of tumor targeting in the anti-tumor efficacy of TAA targeted IL2Rα-IL2 with cleavable linker. As shown in FIG. 9, CD20-targeted IL2Rα-IL2 proteins with cleavable linkers showed significantly better anti-tumor efficacy and tumor free survival compared to non-targeted IL2Rα-IL2 proteins with cleavable linker and CD20 targeted IL2Rα-IL2 with a non-cleavable G4S linker in a MC38/hCD20 mouse model. The bivalent αCD20 with effectorless Fc and non-targeted isotype groups showed no tumor growth control. There was no significant change in body weight in any of the groups of treated mice and the mice did not show any weight loss upon treatment.

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1. An IL2 proprotein, comprising:
(a) a first polypeptide chain comprising, in N- to C-terminal order:
(i) a first amino acid sequence having at least about 95% sequence identity to the amino acid sequence of any one of SEQ ID NOs: 346, 347, 348, 349, 350, and 351;
(ii) a second amino acid sequence comprising a non-cleavable linker sequence;
(iii) a third amino acid sequence having at least about 95% sequence identity to the amino acid sequence of any one of SEQ ID NOs: 3, 4, and 5;
(iv) a fourth amino acid sequence comprising a protease cleavable linker sequence; and
(v) a fifth amino acid sequence having at least about 95% sequence identity to the amino acid sequence of SEQ ID NO: 2; and
(b) a second polypeptide chain comprising, in N- to C-terminal order:
(i) a sixth amino acid sequence having at least about 95% sequence identity to any one of SEQ ID NOs: 346, 347, 348, 349, 350, and 351;
(ii) a seventh amino acid sequence comprising a non-cleavable linker sequence;
(iii) an eighth amino acid sequence having at least about 95% sequence identity to any one of SEQ ID NOs: 3, 4, and 5;
(iv) a ninth amino acid sequence a protease cleavable linker sequence; and
(v) a tenth amino acid sequence having at least about 95% sequence identity to the amino acid sequence of SEQ ID NO: 2.

2. The IL2 proprotein of claim 1, wherein the first polypeptide comprises, N-terminal to the first amino acid sequence, an eleventh amino acid sequence having at least about 95% sequence identity to any one of SEQ ID NOs: 352, 353, 354, and 355.

3.-6. (canceled)

7. The IL2 proprotein of claim 1, wherein the second polypeptide comprises, N-terminal to the first amino acid sequence, a twelfth amino acid sequence having at least about 95% sequence identity to any one of SEQ ID NOs: 352, 353, 354, and 355.

8.-12. (canceled)

13. The IL2 proprotein of claim 1, wherein the first amino acid sequence is the amino acid sequence of any one of SEQ ID NOs: 346, 347, 348, 349, 350, and 351.

14. The IL2 proprotein of claim 13, wherein the first amino acid sequence is the amino acid sequence of SEQ ID NO:350.

15. (canceled)

16. (canceled)

17. The IL2 proprotein of claim 1, wherein the first amino acid sequence is 330 or fewer amino acids in length.

18. (canceled)

19. The IL2 proprotein of claim 1, wherein the sixth amino acid sequence is the amino acid sequence of any one of SEQ ID NOs: 346, 347, 348, 349, 350, and 351.

20. The IL2 proprotein of claim 19, wherein the sixth amino acid sequence is the amino acid sequence of SEQ ID NO:350.

21. (canceled)

22. (canceled)

23. The IL2 proprotein of claim 1, wherein the sixth amino acid sequence is 330 or fewer amino acids in length.

24. (canceled)

25. The IL2 proprotein of claim 1, wherein the second amino acid sequence is the amino acid sequence (GGGGS)ₙ, wherein n is 1, 2, 3, 4, or 5.

26. (canceled)

27. The IL2 proprotein of claim 1, wherein the second amino acid sequence is 15 or fewer amino acids in length.

28. (canceled)

29. (canceled)

30. The IL2 proprotein of claim 1, wherein the seventh amino acid sequence is the amino acid sequence (GGGGS)ₙ, wherein n is 1, 2, 3, 4, or 5.

31. (canceled)

32. The IL2 proprotein of claim 1, wherein the seventh amino acid sequence is 15 or fewer amino acids in length.

33. (canceled)

34. (canceled)

35. The IL2 proprotein of claim 1, wherein the third amino acid sequence is the amino acid sequence of any one of SEQ ID NOs: 3, 4, and 5.

36. (canceled)

37. (canceled)

38. The IL2 proprotein of claim 35, wherein the third amino acid sequence is the amino acid sequence of SEQ ID NO:5.

39. (canceled)

40. (canceled)

41. The IL2 proprotein of claim 1, wherein the third amino acid sequence is 170 or fewer amino acids in length.

42. (canceled)

43. The IL2 proprotein of claim 1, wherein the eighth amino acid sequence is the amino acid sequence of any one of SEQ ID NOs: 3, 4, and 5.

44. (canceled)

45. (canceled)

46. The IL2 proprotein of claim 43, wherein the eighth amino acid sequence is the amino acid sequence of SEQ ID NO:5.

47. (canceled)

48. (canceled)

49. The IL2 proprotein of claim 1, wherein the eighth amino acid sequence is 170 or fewer amino acids in length.

50. (canceled)

51. The IL2 proprotein of claim 1, wherein the fourth amino acid sequence comprises the amino acid sequence HPVGLLAR (SEQ ID NO: 261), VPLSLYSG (SEQ ID NO: 149), ISSGLLS (SEQ ID NO: 64), or PLGLWSQ (SEQ ID NO: 105).

52.-55. (canceled)

56. The IL2 proprotein of claim 1, wherein the fourth amino acid sequence is the amino acid sequence

| (SEQ ID NO: 185) | |
|---|---|
| | GGGISSGLLSGRSDNHGGGISSGLLSGRSDNHGGS, |
| (SEQ ID NO: 189) | |
| | GGSHPVGLLARGGGHPVGLLARGGGHPVGLLARGS, |
| (SEQ ID NO: 339) | |
| | GGGHPVGLLARGGGS, |
| (SEQ ID NO: 338) | |
| | GGGISSGLLSGRSDNHGGGS, |
| | or |
| (SEQ ID NO: 207) | |
| | GGGGSGGGGSGGGGSVPLSLYSGGGSGGSGGSGS. |

57.-61. (canceled)

62. The IL2 proprotein of claim 1, wherein the ninth amino acid sequence comprises the amino acid sequence HPVGLLAR (SEQ ID NO: 261), VPLSLYSG (SEQ ID NO: 149), ISSGLLS (SEQ ID NO: 64), or PLGLWSQ (SEQ ID NO: 105).

63.-66. (canceled)

67. The IL2 proprotein of claim 1, wherein the ninth amino acid sequence is the amino acid sequence

| (SEQ ID NO: 185) | |
|---|---|
| | GGGISSGLLSGRSDNHGGGISSGLLSGRSDNHGGS, |
| (SEQ ID NO: 189) | |
| | GGSHPVGLLARGGGHPVGLLARGGGHPVGLLARGS, |
| (SEQ ID NO: 339) | |
| | GGGHPVGLLARGGGS, |
| (SEQ ID NO: 338) | |
| | GGGISSGLLSGRSDNHGGGS, |
| | or |
| (SEQ ID NO: 207) | |
| | GGGGSGGGGSGGGGSVPLSLYSGGGSGGSGGSGS. |

68.-72. (canceled)

73. The IL2 proprotein of claim 1, wherein the fifth amino acid sequence is the amino acid sequence of SEQ ID NO:2.

74. (canceled)

75. The IL2 proprotein of claim 1, wherein the fifth amino acid sequence is 135 or fewer amino acids in length.

76. (canceled)

77. The IL2 proprotein of claim 1, wherein the tenth amino acid sequence is the amino acid sequence of SEQ ID NO:2.

78. (canceled)

79. The IL2 proprotein of claim 1, wherein the tenth amino acid sequence is 135 or fewer amino acids in length.

80. The IL2 proprotein of claim 1, wherein the first polypeptide chain lacks additional sequences C-terminal to the first amino acid sequence.

81. The IL2 proprotein of claim 1, wherein the first polypeptide chain; (a) lacks additional sequences between the first and second amino acid sequences, (b) lacks additional sequences between the second and third amino acid sequences, (c) lacks additional sequences between the third and fourth amino acid sequences, and (d) lacks additional sequences between the fourth and fifth amino acid sequences.

82.-84. (canceled)

85. The IL2 proprotein of claim 1, wherein the second polypeptide chain lacks additional sequences C-terminal to the sixth amino acid sequence.

86. The IL2 proprotein of claim 1, wherein the second polypeptide chain; (a) lacks additional sequences between the sixth and seventh amino acid sequences, (b) lacks additional sequences between the seventh and eighth amino acid sequences, (c) lacks additional sequences between the eighth and ninth amino acid sequences, and (d) lacks additional sequences between the ninth and tenth amino acid sequences.

87.-89. (canceled)

90. The IL2 proprotein of claim 1, wherein the first polypeptide and the second polypeptide are identical.

91. A nucleic acid or plurality of nucleic acids encoding the IL2 proprotein of claim 1.

92. A host cell engineered to express the IL2 proprotein of claim 1.

93. A method of producing an IL2 proprotein, comprising culturing the host cell of claim 92 and recovering the IL2 proprotein expressed thereby.

94. A pharmaceutical composition comprising the IL2 proprotein of claim 1 and an excipient.

95. A method of treating cancer, comprising administering to a subject in need thereof the IL2 proprotein of claim 1.

96.-133. (canceled)


SEQUENCE LISTING (355 sequences)

SEQ ID NO: 1 - protein, 5 aa, synthetic construct
GGGGS

SEQ ID NO: 2 - protein, 133 aa, Homo sapiens [sequence omitted]

SEQ ID NO: 3 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 4 - protein, 165 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 5 - protein, 219 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 6 - protein, 40 aa, synthetic construct
DEDEDEDEDEDEDEDERPLALWRSDRDRDRDRDRDRDRDR

SEQ ID NO: 7 - protein, 8 aa, synthetic construct
AARGPAIH

SEQ ID NO: 8 - protein, 8 aa, synthetic construct
AAYHLVSQ

SEQ ID NO: 9 - protein, 8 aa, synthetic construct
AGLGISST

SEQ ID NO: 10 - protein, 8 aa, synthetic construct
AGLGVVER

SEQ ID NO: 11 - protein, 4 aa, synthetic construct
ALAL

SEQ ID NO: 12 - protein, 8 aa, synthetic construct
ALFFSSPP

SEQ ID NO: 13 - protein, 8 aa, synthetic construct
ALFKSSFP

SEQ ID NO: 14 - protein, 7 aa, synthetic construct
ALLLALL

SEQ ID NO: 15 - protein, 8 aa, synthetic construct
AQFVLTEG

SEQ ID NO: 16 - protein, 8 aa, synthetic construct
AQNLLGMV

SEQ ID NO: 17 - protein, 8 aa, synthetic construct
AVGLLAPP

SEQ ID NO: 18 - protein, 4 aa, synthetic construct
DAFK

SEQ ID NO: 19 - protein, 4 aa, synthetic construct
DEVD

SEQ ID NO: 20 - protein, 5 aa, synthetic construct
DEVDP

SEQ ID NO: 21 - protein, 6 aa, synthetic construct
DPRSFL

SEQ ID NO: 22 - protein, 8 aa, synthetic construct
DVAQFVLT

SEQ ID NO: 23 - protein, 4 aa, synthetic construct
DVLK

SEQ ID NO: 24 - protein, 8 aa, synthetic construct
DWLYWPGI

SEQ ID NO: 25 - protein, 7 aa, synthetic construct
EDDDDKA

SEQ ID NO: 26 - protein, 6 aa, synthetic construct
  SITE 3: note = Citrulline
  SITE 5: note = Hypofluorous acid modified tyrosine
EPXGYL

SEQ ID NO: 27 - protein, 8 aa, synthetic construct
EPQALAMS

SEQ ID NO: 28 - protein, 8 aa, synthetic construct
ESLPVVAV

SEQ ID NO: 29 - protein, 8 aa, synthetic construct
ESPAYYTA

SEQ ID NO: 30 - protein, 10 aa, synthetic construct
FPRPLGITGL

SEQ ID NO: 31 - protein, 8 aa, synthetic construct
FRLLDWQW

SEQ ID NO: 32 - protein, 4 aa, synthetic construct
GFLG

SEQ ID NO: 33 - protein, 10 aa, synthetic construct
GGAANLVRGG

SEQ ID NO: 34 - protein, 5 aa, synthetic construct
GGGRR

SEQ ID NO: 35 - protein, 8 aa, synthetic construct
GGPRGLPG

SEQ ID NO: 36 - protein, 10 aa, synthetic construct
GGQPSGMWGW

SEQ ID NO: 37 - protein, 7 aa, synthetic construct
GGSIDGR

SEQ ID NO: 38 - protein, 8 aa, synthetic construct
GGWHTGRN

SEQ ID NO: 39 - protein, 5 aa, synthetic construct
GIAGQ

SEQ ID NO: 40 - protein, 6 aa, synthetic construct
GKAFRR

SEQ ID NO: 41 - protein, 8 aa, synthetic construct
GPAGLYAQ

SEQ ID NO: 42 - protein, 8 aa, synthetic construct
GPAGMKGL

SEQ ID NO: 43 - protein, 8 aa, synthetic construct
GPEGLRVG

SEQ ID NO: 44 - protein, 8 aa, synthetic construct
GPLGIAGI

SEQ ID NO: 45 - protein, 7 aa, synthetic construct
GPLGVRG

SEQ ID NO: 46 - protein, 8 aa, synthetic construct
GPQGIAGQ

SEQ ID NO: 47 - protein, 8 aa, synthetic construct
GPQGLLGA

SEQ ID NO: 48 - protein, 6 aa, synthetic construct
GPRSFG

SEQ ID NO: 49 - protein, 7 aa, synthetic construct
GPRSFGL

SEQ ID NO: 50 - protein, 8 aa, synthetic construct
GPSHLVLT

SEQ ID NO: 51 - protein, 10 aa, synthetic construct
GVSQNYPIVG

SEQ ID NO: 52 - protein, 10 aa, synthetic construct
GVVQASCRLA

SEQ ID NO: 53 - protein, 6 aa, synthetic construct
GWEHDG

SEQ ID NO: 54 - protein, 6 aa, synthetic construct
HSSKLQ

SEQ ID NO: 55 - protein, 9 aa, synthetic construct
HSSKLQEDA

SEQ ID NO: 56 - protein, 7 aa, synthetic construct
HSSKLQL

SEQ ID NO: 57 - protein, 8 aa, synthetic construct
HTGRSGAL

SEQ ID NO: 58 - protein, 4 aa, synthetic construct
IDGR

SEQ ID NO: 59 - protein, 4 aa, synthetic construct
IEGR

SEQ ID NO: 60 - protein, 8 aa, synthetic construct
ILPRSPAF

SEQ ID NO: 61 - protein, 8 aa, synthetic construct
IPVSLRSG

SEQ ID NO: 62 - protein, 5 aa, synthetic construct
ISSGL

SEQ ID NO: 63 - protein, 6 aa, synthetic construct
ISSGLL

SEQ ID NO: 64 - protein, 7 aa, synthetic construct
ISSGLLS

SEQ ID NO: 65 - protein, 8 aa, synthetic construct
ISSGLLSS

SEQ ID NO: 66 - protein, 7 aa, synthetic construct
ISSGLSS

SEQ ID NO: 67 - protein, 8 aa, synthetic construct
KGSGDVEG

SEQ ID NO: 68 - protein, 9 aa, synthetic construct
KQEQNPGST

SEQ ID NO: 69 - protein, 8 aa, synthetic construct
KRALGLPG

SEQ ID NO: 70 - protein, 8 aa, synthetic construct
LAAPLGLL

SEQ ID NO: 71 - protein, 9 aa, synthetic construct
LAPLGLQRR

SEQ ID NO: 72 - protein, 8 aa, synthetic construct
LAQKLKSS

SEQ ID NO: 73 - protein, 8 aa, synthetic construct
LAQRLRSS

SEQ ID NO: 74 - protein, 5 aa, synthetic construct
LEATA

SEQ ID NO: 75 - protein, 8 aa, synthetic construct
LKAAPRWA

SEQ ID NO: 76 - protein, 8 aa, synthetic construct
LLAPSHRA

SEQ ID NO: 77 - protein, 8 aa, synthetic construct
LPGGLSPW

SEQ ID NO: 78 - protein, 8 aa, synthetic construct
LSGRSANI

SEQ ID NO: 79 - protein, 8 aa, synthetic construct
LSGRSANP

SEQ ID NO: 80 - protein, 8 aa, synthetic construct
LSGRSDDH

SEQ ID NO: 81 - protein, 8 aa, synthetic construct
LSGRSDIH

SEQ ID NO: 82 - protein, 8 aa, synthetic construct
LSGRSDNH

SEQ ID NO: 83 - protein, 8 aa, synthetic construct
LSGRSDNI

SEQ ID NO: 84 - protein, 8 aa, synthetic construct
LSGRSDNP

SEQ ID NO: 85 - protein, 8 aa, synthetic construct
LSGRSDQG

SEQ ID NO: 86 - protein, 8 aa, synthetic construct
LSGRSDQH

SEQ ID NO: 87 - protein, 8 aa, synthetic construct
LSGRSDTH

SEQ ID NO: 88 - protein, 8 aa, synthetic construct
LSGRSDYH

SEQ ID NO: 89 - protein, 8 aa, synthetic construct
LSGRSGNH

SEQ ID NO: 90 - protein, 10 aa, synthetic construct
LVLASSSFGY

SEQ ID NO: 91 - protein, 8 aa, synthetic construct
MDAFLESS

SEQ ID NO: 92 - protein, 8 aa, synthetic construct
MGLFSEAG

SEQ ID NO: 93 - protein, 8 aa, synthetic construct
MIAPVAYR

SEQ ID NO: 94 - protein, 8 aa, synthetic construct
MVLGRSLL

SEQ ID NO: 95 - protein, 12 aa, synthetic construct
NTLSGRSENHSG

SEQ ID NO: 96 - protein, 12 aa, synthetic construct
NTLSGRSGNHGS

SEQ ID NO: 97 - protein, 8 aa, synthetic construct
PAGLWLDP

SEQ ID NO: 98 - protein, 8 aa, synthetic construct
PGGPAGIG

SEQ ID NO: 99 - protein, 5 aa, synthetic construct
  SITE 4: note = Ethyl phenylalanine
PICFF

SEQ ID NO: 100 - protein, 6 aa, synthetic construct
  SITE 5: note = Methyl alanine
PLGCAG

SEQ ID NO: 101 - protein, 4 aa, synthetic construct
PLGL

SEQ ID NO: 102 - protein, 6 aa, synthetic construct
PLGLAG

SEQ ID NO: 103 - protein, 6 aa, synthetic construct
  VARIANT 6: note = Any amino acid
PLGLAX

SEQ ID NO: 104 - protein, 6 aa, synthetic construct
PLGLWA

SEQ ID NO: 105 - protein, 7 aa, synthetic construct
PLGLWSQ

SEQ ID NO: 106 - protein, 8 aa, synthetic construct
PLTGRSGG

SEQ ID NO: 107 - protein, 5 aa, synthetic construct
PMAKK

SEQ ID NO: 108 - protein, 6 aa, synthetic construct
PPRSFL

SEQ ID NO: 109 - protein, 8 aa, synthetic construct
PRFRIIGG

SEQ ID NO: 110 - protein, 8 aa, synthetic construct
PVGYTSSL

SEQ ID NO: 111 - protein, 8 aa, synthetic construct
PVQPIGPQ

SEQ ID NO: 112 - protein, 8 aa, synthetic construct
QALAMSAI

SEQ ID NO: 113 - protein, 8 aa, synthetic construct
QGRAITFI

SEQ ID NO: 114 - protein, 8 aa, synthetic construct
QNQALRMA

SEQ ID NO: 115 - protein, 4 aa, synthetic construct
RGPA

SEQ ID NO: 116 - protein, 8 aa, synthetic construct
RGPAFNPM

SEQ ID NO: 117 - protein, 8 aa, synthetic construct
RGPATPIM

SEQ ID NO: 118 - protein, 14 aa, synthetic construct
RKSSIIIRMRDVVL

SEQ ID NO: 119 - protein, 7 aa, synthetic construct
RLQLKAC

SEQ ID NO: 120 - protein, 6 aa, synthetic construct
RLQLKL

SEQ ID NO: 121 - protein, 8 aa, synthetic construct
RMHLRSLG

SEQ ID NO: 122 - protein, 8 aa, synthetic construct
RPSPMWAY

SEQ ID NO: 123 - protein, 8 aa, synthetic construct
RQARVVNG

SEQ ID NO: 124 - protein, 134 aa, Homo sapiens
SAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 125 - protein, 8 aa, synthetic construct
SAGFSLPA

SEQ ID NO: 126 - protein, 8 aa, synthetic construct
SAPAVESE

SEQ ID NO: 127 - protein, 8 aa, synthetic construct
SARGPSRW

SEQ ID NO: 128 - protein, 9 aa, synthetic construct
SGEPAYYTA

SEQ ID NO: 129 - protein, 8 aa, synthetic construct
SGGPLGVR

SEQ ID NO: 130 - protein, 10 aa, synthetic construct
SGRIGFLRTA

SEQ ID NO: 131 - protein, 5 aa, synthetic construct
SGRSA

SEQ ID NO: 132 - protein, 9 aa, synthetic construct
SGRSANPRG

SEQ ID NO: 133 - protein, 8 aa, synthetic construct
SMLRSMPL

SEQ ID NO: 134 - protein, 8 aa, synthetic construct
SPLPLRVP

SEQ ID NO: 135 - protein, 8 aa, synthetic construct
SPLTGRSG

SEQ ID NO: 136 - protein, 8 aa, synthetic construct
SPRSIMLA

SEQ ID NO: 137 - protein, 8 aa, synthetic construct
SSRGPAYL

SEQ ID NO: 138 - protein, 9 aa, synthetic construct
SSRHRRALD

SEQ ID NO: 139 - protein, 15 aa, synthetic construct
SSSFDKGKYKKGDDA

SEQ ID NO: 140 - protein, 15 aa, synthetic construct
SSSFDKGKYKRGDDA

SEQ ID NO: 141 - protein, 8 aa, synthetic construct
STFPFGMF

SEQ ID NO: 142 - protein, 8 aa, synthetic construct
TARGPSFK

SEQ ID NO: 143 - protein, 8 aa, synthetic construct
TGRGPSWV

SEQ ID NO: 144 - protein, 8 aa, synthetic construct
TSGRSANP

SEQ ID NO: 145 - protein, 12 aa, synthetic construct
TSTSGRSANPRG

SEQ ID NO: 146 - protein, 8 aa, synthetic construct
VAGRSMRP

SEQ ID NO: 147 - protein, 8 aa, synthetic construct
VAQFVLTE

SEQ ID NO: 148 - protein, 10 aa, synthetic construct
VHMPLGFLGP

SEQ ID NO: 149 - protein, 8 aa, synthetic construct
VPLSLYSG

SEQ ID NO: 150 - protein, 8 aa, synthetic construct
VVPEGRRS

SEQ ID NO: 151 - protein, 8 aa, synthetic construct
WATPRPMR

SEQ ID NO: 152 - protein, 8 aa, synthetic construct
YGAGLGVV

SEQ ID NO: 153 - protein, 5 aa, synthetic construct
GGGGS

SEQ ID NO: 154 - protein, 4 aa, synthetic construct
GGGS

SEQ ID NO: 155 - protein, 5 aa, synthetic construct
GSGGS

SEQ ID NO: 156 - protein, 10 aa, synthetic construct
GGGGSGGGGS

SEQ ID NO: 157 - protein, 15 aa, synthetic construct
GGGGSGGGGSGGGGS

SEQ ID NO: 158 - protein, 20 aa, synthetic construct
GGGGSGGGGSGGGGSGGGGS

SEQ ID NO: 159 - protein, 15 aa, synthetic construct
GGGKSGGGKSGGGKS

SEQ ID NO: 160 - protein, 15 aa, synthetic construct
GGGKSGGKGSGKGGS

SEQ ID NO: 161 - protein, 4 aa, synthetic construct
GGGS

SEQ ID NO: 162 - protein, 5 aa, synthetic construct
GGGSG

SEQ ID NO: 163 - protein, 15 aa, synthetic construct
GGKGSGGKGSGGKGS

SEQ ID NO: 164 - protein, 13 aa, synthetic construct
GGSGGGGSGGGGS

SEQ ID NO: 165 - protein, 6 aa, synthetic construct
GGSGGS

SEQ ID NO: 166 - protein, 11 aa, synthetic construct
GGSGGSGGSGS

SEQ ID NO: 167 - protein, 5 aa, synthetic construct
GSGGG

SEQ ID NO: 168 - protein, 5 aa, synthetic construct
GSGSG

SEQ ID NO: 169 - protein, 4 aa, synthetic construct
GSSG

SEQ ID NO: 170 - protein, 10 aa, synthetic construct
GSSGGSGGSG

SEQ ID NO: 171 - protein, 11 aa, synthetic construct
GSSGGSGGSGG

SEQ ID NO: 172 - protein, 12 aa, synthetic construct
GSSGGSGGSGGS

SEQ ID NO: 173 - protein, 13 aa, synthetic construct
GSSGGSGGSGGSG

SEQ ID NO: 174 - protein, 16 aa, synthetic construct
GSSGGSGGSGGSGGGS

SEQ ID NO: 175 - protein, 11 aa, synthetic construct
GSSGGSGGSGS

SEQ ID NO: 176 - protein, 5 aa, synthetic construct
GSSGT

SEQ ID NO: 177 - protein, 5 aa, synthetic construct
GSSSG

SEQ ID NO: 178 - protein, 6 aa, synthetic construct
QGQSGQ

SEQ ID NO: 179 - protein, 7 aa, synthetic construct
QGQSGQG

SEQ ID NO: 180 - protein, 6 aa, synthetic construct
QGQSGS

SEQ ID NO: 181 - protein, 4 aa, synthetic construct
QSGQ

SEQ ID NO: 182 - protein, 5 aa, synthetic construct
QSGQG

SEQ ID NO: 183 - protein, 4 aa, synthetic construct
QSGS

SEQ ID NO: 184 - protein, 4 aa, synthetic construct
SGQG

SEQ ID NO: 185 - protein, 35 aa, synthetic construct
GGGISSGLLSGRSDNHGGGISSGLLSGRSDNHGGS

SEQ ID NO: 186 - protein, 70 aa, synthetic construct
GGGISSGLLSGRSDNHGGGISSGLLSGRSDNHGGSGGGISSGLLSGRSDNHGGGISSGLLSGRSDNHGGS

SEQ ID NO: 187 - protein, 35 aa, synthetic construct
GGSGGSIPVSLRSGGGISSGLLSGRSDNHGGSGGS

SEQ ID NO: 188 - protein, 35 aa, synthetic construct
GGSGGSVPLSLYSGGGISSGLLSGRSDNHGGSGGS

SEQ ID NO: 189 - protein, 35 aa, synthetic construct
GGSHPVGLLARGGGHPVGLLARGGGHPVGLLARGS

SEQ ID NO: 190 - protein, 36 aa, synthetic construct
GGSHPVGLLARGGGHPVGLLARGGSGRSAGGSGRSA

SEQ ID NO: 191 - protein, 18 aa, synthetic construct
AVGLLAPPGGLSGRSANI

SEQ ID NO: 192 - protein, 18 aa, synthetic construct
AVGLLAPPGGLSGRSANP

SEQ ID NO: 193 - protein, 18 aa, synthetic construct
AVGLLAPPGGLSGRSDDH

SEQ ID NO: 194 - protein, 18 aa, synthetic construct
AVGLLAPPGGLSGRSDIH

SEQ ID NO: 195 - protein, 18 aa, synthetic construct
AVGLLAPPGGLSGRSDNH

SEQ ID NO: 196 - protein, 18 aa, synthetic construct
AVGLLAPPGGLSGRSDNI

SEQ ID NO: 197 - protein, 18 aa, synthetic construct
AVGLLAPPGGLSGRSDNP

SEQ ID NO: 198 - protein, 18 aa, synthetic construct
AVGLLAPPGGLSGRSDQH

SEQ ID NO: 199 - protein, 18 aa, synthetic construct
AVGLLAPPGGLSGRSDTH

SEQ ID NO: 200 - protein, 18 aa, synthetic construct
AVGLLAPPGGLSGRSDYH

SEQ ID NO: 201 - protein, 22 aa, synthetic construct
AVGLLAPPGGTSTSGRSANPRG

SEQ ID NO: 202 - protein, 17 aa, synthetic construct
AVGLLAPPSGRSANPRG

SEQ ID NO: 203 - protein, 18 aa, synthetic construct
AVGLLAPPTSGRSANPRG

SEQ ID NO: 204 - protein, 30 aa, synthetic construct
GGALFKSSFPGPAGLYAQPLAQKLKSSGGK

SEQ ID NO: 205 - protein, 33 aa, synthetic construct
GGGGSGGGGSGGGGSFVGGTGGGGSGGGGSGGS

SEQ ID NO: 206 - protein, 34 aa, synthetic construct
GGGGSGGGGSGGGGSISSGLLSGRSDNHGGSGGS

SEQ ID NO: 207 - protein, 34 aa, synthetic construct
GGGGSGGGGSGGGGSVPLSLYSGGGSGGSGGSGS

SEQ ID NO: 208 - protein, 35 aa, synthetic construct
GGGGSGGGGSGPLGLWSQGGGGSGGGGSGGGGSGG

SEQ ID NO: 209 - protein, 33 aa, synthetic construct
GGGGSGGGGSKKAAPGGGGSGGGGSGGGGSGGS

SEQ ID NO: 210 - protein, 33 aa, synthetic construct
GGGGSGGGGSKKAAPVNGGGGGSGGGGSGGGGS

SEQ ID NO: 211 - protein, 33 aa, synthetic construct
GGGGSGGGGSPMAKKGGGGSGGGGSGGGGSGGS

SEQ ID NO: 212 - protein, 33 aa, synthetic construct
GGGGSGGGGSPMAKKVNGGGGGSGGGGSGGGGS

SEQ ID NO: 213 - protein, 33 aa, synthetic construct
GGGGSGGGGSQARAKGGGGSGGGGSGGGGSGGS

SEQ ID NO: 214 - protein, 33 aa, synthetic construct
GGGGSGGGGSQARAKVNGGGGGSGGGGSGGGGS

SEQ ID NO: 215 - protein, 33 aa, synthetic construct
GGGGSGGGGSRQARVVNGGGGGSGGGGSGGGGS

SEQ ID NO: 216 - protein, 41 aa, synthetic construct
GGGGSGGGGSRQARVVNGGGGGSVPLSLYSGGGGGSGGGGS

SEQ ID NO: 217 - protein, 36 aa, synthetic construct
GGGGSGGGGSRQARVVNSVPLSLYSGGGGGSGGGGS

SEQ ID NO: 218 - protein, 33 aa, synthetic construct
GGGGSGGGGSVHMPLGFLGPGGGGSGGGGSGGS

SEQ ID NO: 219 - protein, 28 aa, synthetic construct
GGGGSVHMPLGFLGPGRSRGSFPGGGGS

SEQ ID NO: 220 - protein, 33 aa, synthetic construct
GGGGSVHMPLGFLGPPMAKKGGGGSGGGGSGGS

SEQ ID NO: 221 - protein, 32 aa, synthetic construct
GGGGSVHMPLGFLGPRQARVVNGGGGSGGGGS

SEQ ID NO: 222 - protein, 34 aa, synthetic construct
GGGGSVHMPLGFLGPRQARVVNGGGGSGGGGSGG

SEQ ID NO: 223 - protein, 30 aa, synthetic construct
GGPLAQKLKSSALFKSSFPGPAGLYAQGGR

SEQ ID NO: 224 - protein, 19 aa, synthetic construct
GLSGRSDNHGGAVGLLAPP

SEQ ID NO: 225 - protein, 21 aa, synthetic construct
GLSGRSDNHGGVHMPLGFLGP

SEQ ID NO: 226 - protein, 13 aa, synthetic construct
ISSGLLSGRSANI

SEQ ID NO: 227 - protein, 13 aa, synthetic construct
ISSGLLSGRSANP

SEQ ID NO: 228 - protein, 15 aa, synthetic construct
ISSGLLSGRSANPRG

SEQ ID NO: 229 - protein, 13 aa, synthetic construct
ISSGLLSGRSDDH

SEQ ID NO: 230 - protein, 13 aa, synthetic construct
ISSGLLSGRSDIH

SEQ ID NO: 231 - protein, 13 aa, synthetic construct
ISSGLLSGRSDNH

SEQ ID NO: 232 - protein, 13 aa, synthetic construct
ISSGLLSGRSDNI

SEQ ID NO: 233 - protein, 13 aa, synthetic construct
ISSGLLSGRSDNP

SEQ ID NO: 234 - protein, 13 aa, synthetic construct
ISSGLLSGRSDQH

SEQ ID NO: 235 - protein, 13 aa, synthetic construct
ISSGLLSGRSDTH

SEQ ID NO: 236 - protein, 13 aa, synthetic construct
ISSGLLSGRSDYH

SEQ ID NO: 237 - protein, 13 aa, synthetic construct
ISSGLLSGRSGNH

SEQ ID NO: 238 - protein, 22 aa, synthetic construct
ISSGLLSSGGSGGSLSGRSDNH

SEQ ID NO: 239 - protein, 22 aa, synthetic construct
ISSGLLSSGGSGGSLSGRSGNH

SEQ ID NO: 240 - protein, 30 aa, synthetic construct
KGGPGGPAGIGPLAQRLRSSALFKSSFPGR

SEQ ID NO: 241 - protein, 30 aa, synthetic construct
KSGPGGPAGIGALFFSSPPLAQKLKSSGGR

SEQ ID NO: 242 - protein, 18 aa, synthetic construct
LSGRSDNHGGAVGLLAPP

SEQ ID NO: 243 - protein, 22 aa, synthetic construct
LSGRSDNHGGSGGSISSGLLSS

SEQ ID NO: 244 - protein, 22 aa, synthetic construct
LSGRSDNHGGSGGSQNQALRMA

SEQ ID NO: 245 - protein, 20 aa, synthetic construct
LSGRSDNHGGVHMPLGFLGP

SEQ ID NO: 246 - protein, 22 aa, synthetic construct
LSGRSGNHGGSGGSISSGLLSS

SEQ ID NO: 247 - protein, 22 aa, synthetic construct
LSGRSGNHGGSGGSQNQALRMA

SEQ ID NO: 248 - protein, 22 aa, synthetic construct
QNQALRMAGGSGGSLSGRSDNH

SEQ ID NO: 249 - protein, 22 aa, synthetic construct
QNQALRMAGGSGGSLSGRSGNH

SEQ ID NO: 250 - protein, 30 aa, synthetic construct
RGGALFKSSFPLAQKLKSSGPAGLYAQGGK

SEQ ID NO: 251 - protein, 30 aa, synthetic construct
RGGGPAGLYAQPLAQKLKSSALFKSSFPGG

SEQ ID NO: 252 - protein, 30 aa, synthetic construct
SGGFPRSGGSFNPRTFGSKRKRRGSRGGGG

SEQ ID NO: 253 - protein, 30 aa, synthetic construct
SGPLAQKLKSSGPAGLYAQALFKSSFPGSK

SEQ ID NO: 254 - protein, 22 aa, synthetic construct
TSTSGRSANPRGGGAVGLLAPP

SEQ ID NO: 255 - protein, 24 aa, synthetic construct
TSTSGRSANPRGGGVHMPLGFLGP

SEQ ID NO: 256 - protein, 20 aa, synthetic construct
VHMPLGFLGPGGLSGRSDNH

SEQ ID NO: 257 - protein, 24 aa, synthetic construct
VHMPLGFLGPGGTSTSGRSANPRG

SEQ ID NO: 258 - protein, 21 aa, synthetic construct
SGRSAGGGSGRSAGGGSGRSA

SEQ ID NO: 259 - protein, 35 aa, synthetic construct
HPVGLLARGGGHPVGLLARGGGSGRSAGGGSGRSA

SEQ ID NO: 260 - protein, 8 aa, synthetic construct
GPLGVRGK

SEQ ID NO: 261 - protein, 8 aa, synthetic construct
HPVGLLAR

SEQ ID NO: 262 - protein, 8 aa, synthetic construct
VPMSMRGG

SEQ ID NO: 263 - protein, 8 aa, synthetic construct
RPFSMIMG

SEQ ID NO: 264 - protein, 8 aa, synthetic construct
VPLSLTMG

SEQ ID NO: 265 - protein, 8 aa, synthetic construct
IPESLRAG

SEQ ID NO: 266 - protein, 11 aa, synthetic construct
  VARIANT 1..10: note = This region may encompass 1-10 residues
GGGGGGGGGGS

SEQ ID NO: 267 - protein, 11 aa, synthetic construct
  VARIANT 2..11: note = This region may encompass 1-10 residues
SGGGGGGGGGG

SEQ ID NO: 268 - protein, 4 aa, synthetic construct
GGGG

SEQ ID NO: 269 - protein, 5 aa, synthetic construct
GGGGG

SEQ ID NO: 270 - protein, 6 aa, synthetic construct
GGGGGG

SEQ ID NO: 271 - protein, 7 aa, synthetic construct
GGGGGGG

SEQ ID NO: 272 - protein, 8 aa, synthetic construct
GGGGGGGG

SEQ ID NO: 273 - protein, 9 aa, synthetic construct
GGGGGGGGG

SEQ ID NO: 274 - protein, 35 aa, synthetic construct
GGGGSLALGPGGGGGSLALGPGGGGGSLALGPGGS

SEQ ID NO: 275 - protein, 25 aa, synthetic construct
GGGGSGGGGSGGGGSGGGGSGGGGS

SEQ ID NO: 276 - protein, 5 aa, synthetic construct
ADAAP

SEQ ID NO: 277 - protein, 11 aa, synthetic construct
ADAAPTVSIFP

SEQ ID NO: 278 - protein, 12 aa, synthetic construct
ADAAPTVSIFPP

SEQ ID NO: 279 - protein, 6 aa, synthetic construct
AKTTAP

SEQ ID NO: 280 - protein, 13 aa, synthetic construct
AKTTAPSVYPLAP

SEQ ID NO: 281 - protein, 17 aa, synthetic construct
AKTTPKLEEGEFSEARV

SEQ ID NO: 282 - protein, 9 aa, synthetic construct
AKTTPKLGG

SEQ ID NO: 283 - protein, 6 aa, synthetic construct
AKTTPP

SEQ ID NO: 284 - protein, 13 aa, synthetic construct
AKTTPPSVTPLAP

SEQ ID NO: 285 - protein, 6 aa, synthetic construct
ASTKGP

SEQ ID NO: 286 - protein, 26 aa, synthetic construct
ASTKGPSVFPLAPASTKGPSVFPLAP

SEQ ID NO: 287 - protein, 14 aa, synthetic construct
EGKSSGSGSESKST

SEQ ID NO: 288 - protein, 15 aa, synthetic construct
GEGESGEGESGEGES

SEQ ID NO: 289 - protein, 20 aa, synthetic construct
GEGESGEGESGEGESGEGES

SEQ ID NO: 290 - protein, 15 aa, synthetic construct
GEGGSGEGGSGEGGS

SEQ ID NO: 291 - protein, 15 aa, synthetic construct
GENKVEYAPALMALS

SEQ ID NO: 292 - protein, 15 aa, synthetic construct
GGEGSGGEGSGGEGS

SEQ ID NO: 293 - protein, 15 aa, synthetic construct
GGGESGGEGSGEGGS

SEQ ID NO: 294 - protein, 15 aa, synthetic construct
GGGESGGGESGGGES

SEQ ID NO: 295 - protein, 4 aa, synthetic construct
GGSG

SEQ ID NO: 296 - protein, 5 aa, synthetic construct
GGSGG

SEQ ID NO: 297 - protein, 9 aa, synthetic construct
GGSGGGGSG

SEQ ID NO: 298 - protein, 15 aa, synthetic construct
GHEAAAVMQVQYPAS

SEQ ID NO: 299 - protein, 15 aa, synthetic construct
GKGGSGKGGSGKGGS

SEQ ID NO: 300 - protein, 15 aa, synthetic construct
GKGKSGKGKSGKGKS

SEQ ID NO: 301 - protein, 20 aa, synthetic construct
GKGKSGKGKSGKGKSGKGKS

SEQ ID NO: 302 - protein, 15 aa, synthetic construct
GKPGSGKPGSGKPGS

SEQ ID NO: 303 - protein, 21 aa, synthetic construct
GKPGSGKPGSGKPGSGKPSGS

SEQ ID NO: 304 - protein, 15 aa, synthetic construct
GPAKELTPLKEAKVS

SEQ ID NO: 305 - protein, 12 aa, synthetic construct
GSAGSAAGSGEF

SEQ ID NO: 306 - protein, 18 aa, synthetic construct
GSTSGSGKPGSGEGSTKG

SEQ ID NO: 307 - protein, 19 aa, synthetic construct
GTAAAGAGAAGGAAAGAAG

SEQ ID NO: 308 - protein, 19 aa, synthetic construct
GTSGSSGSGSGGSGSGGGG

SEQ ID NO: 309 - protein, 14 aa, synthetic construct
IRPRAIGGSKPRVA

SEQ ID NO: 310 - protein, 18 aa, synthetic construct
KESGSVSSEQLAQFRSLD

SEQ ID NO: 311 - protein, 15 aa, synthetic construct
KTTPKLEEGEFSEAR

SEQ ID NO: 312 - protein, 19 aa, synthetic construct
PRGASKSGSASQTGSAPGS

SEQ ID NO: 313 - protein, 6 aa, synthetic construct
QPKAAP

SEQ ID NO: 314 - protein, 13 aa, synthetic construct
QPKAAPSVTLFPP

SEQ ID NO: 315 - protein, 27 aa, synthetic construct
RADAAAAGGGGSGGGGSGGGGSGGGGS

SEQ ID NO: 316 - protein, 12 aa, synthetic construct
RADAAAAGGPGS

SEQ ID NO: 317 - protein, 6 aa, synthetic construct
RADAAP

SEQ ID NO: 318 - protein, 9 aa, synthetic construct
RADAAPTVS

SEQ ID NO: 319 - protein, 6 aa, synthetic construct
SAKTTP

SEQ ID NO: 320 - protein, 18 aa, synthetic construct
SAKTTPKLEEGEFSEARV

SEQ ID NO: 321 - protein, 10 aa, synthetic construct
SAKTTPKLGG

SEQ ID NO: 322 - protein, 14 aa, synthetic construct
STAGDTHLGGEDFD

SEQ ID NO: 323 - protein, 5 aa, synthetic construct
TVAAP

SEQ ID NO: 324 - protein, 12 aa, synthetic construct
TVAAPSVFIFPP

SEQ ID NO: 325 - protein, 24 aa, synthetic construct
TVAAPSVFIFPPTVAAPSVFIFPP

SEQ ID NO: 326 - protein, 20 aa, synthetic construct
EPKSCDKTHTCPPCPAPPVA

SEQ ID NO: 327 - protein, 17 aa, synthetic construct
ESKYGPPCPPCPAPPVA

SEQ ID NO: 328 - protein, 15 aa, synthetic construct
CPPCPAPGGGGPSVF

SEQ ID NO: 329 - protein, 14 aa, synthetic construct
CPPCPAPGGGPSVF

SEQ ID NO: 330 - protein, 13 aa, synthetic construct
CPPCPAPGGPSVF

SEQ ID NO: 331 - protein, 12 aa, synthetic construct
CPPCPAPGPSVF

SEQ ID NO: 332 - protein, 231 aa, synthetic construct
DKRVESKYGPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 333 - protein, 235 aa, synthetic construct
DKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 334 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 335 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 336 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 337 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 338 - protein, 20 aa, synthetic construct
GGGISSGLLSGRSDNHGGGS

SEQ ID NO: 339 - protein, 15 aa, synthetic construct
GGGHPVGLLARGGGS

SEQ ID NO: 340 - protein, 36 aa, synthetic construct
GGGSGGGSGGGGISSGLLSGRSDNHGGGSGGGSGGS

SEQ ID NO: 341 - protein, 36 aa, synthetic construct
GGGGISSGLLSGRSDNHGGGISSGLLSGRSDNHGGS

SEQ ID NO: 342 - protein, 36 aa, synthetic construct
GGGSGGSIPVSLRSGGGISSGLLSGRSDNHGGSGGS

SEQ ID NO: 343 - protein, 36 aa, synthetic construct
GGGSGGSVPLSLYSGGGISSGLLSGRSDNHGGSGGS

SEQ ID NO: 344 - protein, 36 aa, synthetic construct
GGGSHPVGLLARGGGHPVGLLARGGGHPVGLLARGS

SEQ ID NO: 345 - protein, 36 aa, synthetic construct
GGGSHPVGLLARGGGHPVGLLARGGSGRSAGGSGRS

SEQ ID NO: 346 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 347 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 348 - protein, 279 aa, synthetic construct
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 349 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 350 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 351 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 352 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 353 - protein, 98 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 354 - protein, 98 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 355 - protein, 98 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV